(19) United States
(12) Patent Application Publication
(10) Pub. No.: US 2023/0044432 A1
(43) Pub. Date: Feb. 9, 2023

(54) Pooled Crispr Inverse PCR Sequencing (PCIP-Seq): Simultaneous Sequencing of Viral Insertion Points and the Integrated Viral Genomes with Long Reads

(71) Applicants: UNIVERSITÉ DE LIÈGE, Liège (BE); INSTITUT JULES BORDET, Bruxelles (BE)

(72) Inventors: Keith Durkin, Liège (BE); Maria Artesi, Liège (BE); Anne Van Den Broeke, Dworp (BE); Vincent Bours, Liège (BE); Vincent Hahaut, Liège (BE); Michel Georges, Liège (BE)

(21) Appl. No.: 17/781,980

(22) PCT Filed: Dec. 3, 2020
(86) PCT No.: PCT/EP2020/084557

§ 371 Date: Jun. 2, 2022

Related U.S. Application Data
(60) Provisional application No. 62/942,972, filed on Dec. 3, 2019.

(51) Int. Cl. C12Q 1/6806; C12Q 1/70; C12N 15/113

(52) U.S. Cl. CPC C12Q 1/6806; C12Q 1/708; C12N 15/113; C12N 2310/20; C12Q 2521/501; C12Q 2525/307; C12Q 2531/113; C12Q 2535/122; C12Q 2565/631

(57) ABSTRACT

The present invention relates to a method for detecting an integration pattern of a virus in a host genome. In particular, a method is provided encompassing selective cleavage of circularized DNA fragments carrying viral DNA with an RNA-guided endonuclease and at least one guide RNA or at least one pool of guide RNAs, followed by inverse PCR, in particular inverse long-range PCR, and sequencing. The invention further relates to kits for performing the method and application of the method.

### CROSS REFERENCE TO RELATED APPLICATIONS

This application is a national phase entry under 35 U.S.C. § 371 of International Patent Application PCT/EP2020/084557, filed Dec. 3, 2020, designating the United States of America and published in English as International Patent Publication WO 2021/110878 on Jun. 10, 2021, which claims the benefit under Article 8 of the Patent Cooperation Treaty to U.S. Patent Application Ser. No. 62/942,972, filed Dec. 3, 2019, the entireties of which are hereby incorporated by reference.

### TECHNICAL FIELD

The present invention relates to a method for detecting an integration pattern of a virus in a host genome, tools for performing the method and applications thereof.

### BACKGROUND

The integration of viral DNA into the host genome is a defining feature of the retroviral life cycle, irreversibly linking provirus and cell. This intimate association facilitates viral persistence and replication in somatic cells, and with integration into germ cells bequeaths the provirus to subsequent generations. Considerable effort has been expended to understand patterns of proviral integration, both from a basic virology stand point, and due to the use of retroviral vectors in gene therapy¹. The application of next generation sequencing (NGS) over the last ˜10 years has had a dramatic impact on our ability to explore the landscape of retroviral integration for both exogenous and endogenous retroviruses. Methods based on ligation mediated PCR and Illumina sequencing have facilitated the identification of hundreds of thousands of insertion sites in exogenous viruses such as Human T-cell leukemia virus-1 (HTLV-1)² and Human immunodeficiency virus (HIV-1)³⁻⁶. These techniques have shown that in HTLV-1², Bovine Leukemia Virus (BLV)⁷ and Avian Leukosis Virus (ALV)⁸ integration sites are not random, pointing to clonal selection. In HIV-1 it has also become apparent that provirus integration can drive clonal expansion^{3,4,8,8}, magnifying the HIV-1 reservoir and placing a major road block in the way of a complete cure.

Current methods based on short-read (high throughput) sequencing identify the insertion point, but the provirus itself is largely unexplored. Whether variation in the provirus influences the fate of the clone remains difficult to investigate. Using long range PCR it has been shown that proviruses in HTLV-1 induced Adult T-cell leukemia (ATL) are frequently (˜45%) defective¹⁰, although the abundance of defective proviruses within asymptomatic HTLV-1 carriers has not been systematically investigated. Recently, there has been a concerted effort to better understand the structure of HIV-1 proviruses in the latent reservoir. Methods such as Full-Length Individual Proviral Sequencing (FLIPS) have been developed to identify functional proviruses¹¹ but without identifying the provirus integration site. More recently matched integration site and proviral sequencing (MIP-Seq) has allowed the sequence of individual proviruses to be linked to integration site in the genome⁶. However, this method relies on whole genome amplification of isolated HIV-1 genomes, with separate reactions to identify the integration site and sequence the associated provirus⁶. As a result, this method is quite labor intensive limiting the number of proviruses one can reasonably interrogate.

Retroviruses are primarily associated with the diseases they provoke through the infection of somatic cells. Over the course of evolutionary time they have also played a major role in shaping the genome. Retroviral invasion of the germ line has occurred multiple times, resulting in the remarkable fact that endogenous retrovirus (ERV)-like elements comprise a larger proportion of the human genome (8%) than protein coding sequences (˜1.5%)¹². With the availability of multiple vertebrate genome assemblies, much of the focus has been on comparison of ERVs between species. However, single genomes represent a fraction of the variation within a species, prompting some to take a population approach to investigate ERV-host genome variation¹³. While capable of identifying polymorphic ERVs in the population, approaches relying on conventional paired-end libraries and short reads cannot capture the sequence of the provirus beyond the first few hundred bases of the proviral long terminal repeat (LTR), leaving the variation within uncharted.

In contrast to retroviruses, papillomaviruses do not integrate into the host genome as part of their lifecycle. Human papillomavirus (HPV) is usually present in the cell as a multi copy circular episome (˜8 kb in size), however in a small fraction of infections, it can integrate into the host genome leading to the dysregulation of the viral oncogenes E6 and E7¹⁴. Genome wide profiling of HPV integration sites via capture probes and Illumina sequencing has also identified hotspots of integration indicating that disruption of host genes may also play a role in driving clonal expansion¹⁵. As a consequence, HPV integration is a risk factor for the development of cervical carcinoma¹⁶.

HPV accounts for >95% of cervical carcinoma and ˜70% of oropharyngeal carcinoma⁵². While infection with a high-risk HPV strain (HPV16 & HPV18) is generally necessary for the development of cervical cancer, it is not sufficient⁴¹. The progression towards cancer is driven by a combination of both viral and host factors, as a result, a greater understanding of both is required to identify high risk infections⁴¹.

The HPV vaccine will cut the rate of cervical cancer in vaccinated women by ˜75%, however it will take 20 to 30 years for the full impact of vaccination to become apparent⁶⁴. Additionally, vaccination uptake varies widely, with the Belgian French speaking community only having a 36% uptake in 2018⁶⁵. As consequence HPV induced cervical cancer will remain a major health issue in the medium term and the cause of a nontrivial number of cancers into the foreseeable future.

The centrality of HPV integration in carcinogenesis makes a deeper understanding of the process a priority, both to understand the basic biology behind HPV induced cervical cancer, but also because of its potential as a biomarker to identify high risk cases sooner. The study of HPV integration is hampered by the unpredictability of the breakpoint sites in the integrated HPV genome. This limits the applicability of approaches based on ligation mediated PCR and short read sequencing. Techniques such as real-time PCR can identify HPV infections, but cannot identify integrations associated with clonal expansion. Biotin capture probes and Illumina sequencing have provided an unbiased genome wide picture of integration sites in cervical carcinomas, hinting at potential hot spots of integration¹⁵. However, this technique is not suited to exploring precancerous stages, where only a small fraction of the cells carries integrated virus. Looking beyond integration sites, work on HPV16 using a targeted sequencing approach has shown that conservation of the HPV E7 gene is critical for carcinogenesis⁶⁶.

The application of NGS as well as Sanger sequencing before, has had a large impact on our understanding of both exogenous and endogenous proviruses. The development of long-read sequencing, linked-read technologies and associated computational tools¹⁷ have the potential to explore questions inaccessible to short reads. Groups investigating Long interspersed nuclear elements-1 (LINE-1) insertions¹⁶ and the koala retrovirus, KoRV¹⁹ have highlighted this potential and described techniques utilizing the Oxford Nanopore and PacBio platforms, to investigate insertion sites and retroelement structure.

### SUMMARY OF THE INVENTION

To more fully exploit the potential of long reads we developed Pooled CRISPR Inverse PCR sequencing (PCIP-seq), a method that leverages selective cleavage of circularized DNA fragments carrying proviral DNA/integrated viral DNA with CRISPR guide RNAs or a pool of CRISPR guide RNAs, followed by inverse long-range PCR and multiplexed sequencing, such as on the Oxford Nanopore MinION platform. Using this approach, we can now simultaneously identify the integration site and track clone abundance while also sequencing the provirus/viral DNA inserted at that position. We have successfully applied the technique to the retroviruses HTLV-1, HIV-1 and BLV, endogenous retroviruses in cattle and sheep as well as HPV18 and HPV16.

In an aspect, the invention provides a method for detecting an integration pattern of human papillomavirus (HPV) in genomic DNA of a subject, said method comprising:

(a) fragmenting genomic DNA isolated from a sample of the subject;

(b) circularizing the DNA fragments to generate circular DNA;

(c) removing non-circularized DNA fragments;

(d) linearizing the circular DNA using an RNA-guided DNA endonuclease and at least one guide RNA or at least one pool of guide RNAs, which target a region in the viral genome, to generate linearized DNA molecules;

(e) amplifying the linearized DNA molecules by an inverse amplification reaction using a pair of primers arranged about and oriented outwardly with respect to the linearization site;

(f) sequencing the amplified DNA;

(g) mapping the sequenced DNA to human genomic DNA sequence; and

(h) optionally mapping the sequenced DNA to the HPV genome.

The invention also provides for a kit for detecting an integration pattern of human papillomavirus (HPV) in genomic DNA of a subject according to the method of of the invention, said kit comprising:
at least one first guide RNA or at least one first pool of guide RNAs, which target a first region in the viral genome, preferably wherein said first region of the viral DNA comprises E6 gene and/or E7 gene; and/or, preferably and,
a pair of primers arranged about and oriented outwardly with respect to a first linearization site in the viral genome defined by said at least one first guide RNA or at least first one pool of guide RNAs.

A further aspect relates to a method for monitoring the progression of a human papillomavirus (HPV) infection in a subject comprising:
detecting an integration pattern of human papillomavirus (HPV) in genomic DNA isolated from a sample of the subject according to the method of the invention; and
comparing said integration pattern with an integration pattern of HPV in genomic DNA isolated from a sample of the subject at an earlier point in time.

A further aspect relates to a method for assessing a risk of having or developing a cancer in a subject comprising:
detecting an integration pattern of human papillomavirus (HPV) in genomic DNA of the subject according to the method of the invention; and
determining whether the integration pattern predisposes the subject to cancer or cancer development. These and further aspects and preferred embodiments of the invention are described in the following sections and in the appended claims. The subject-matter of the appended claims is hereby specifically incorporated in this specification.

### BRIEF DESCRIPTION OF THE FIGURES

The teaching of the application is illustrated by the following Figures which are to be considered as illustrative only and do not in any way limit the scope of the claims.

FIGS. 1A-1D. Overview of the PCIP-seq method (FIG. 1A) Simplified outline of method (FIG. 1B) A pool of CRISPR guide-RNAs targets each region, the region is flanked by PCR primers. Guides and primers adjacent to 5′ & 3′ LTRs are multiplexed. (FIG. 1C) As the region between the PCR primers is not sequenced we created two sets of guides and primers. Following circularization, the sample is split, with CRISPR mediated cleavage and PCR occurring separately for each set. After PCR the products of the two sets of guides and primers are combined for sequencing. (FIG. 1D) Screen shot from the Integrative Genomics Viewer (IGV) showing a small fraction of the resultant reads (grey bars) mapped to the provirus, coverage is shown on top, coverage drops close to the 5′ and 3′ ends are regions flanked by primers.

FIGS. 2A-2E. PCIP-seq applied to ATL (FIG. 2A) In ATL100 both Illumina and Nanopore based methods show a single predominant insertion site (FIG. 2B) Screen shot from IGV shows a ˜16 kb window with the provirus insertion site in the tumor clone identified via PCIP-seq and ligation mediated PCR with Illumina sequencing (FIG. 2C) PCIP-seq reads in IGV show a ˜3,600 bp deletion in the provirus, confirmed via long range PCR and Illumina sequencing. (FIG. 2D) The ATL2 tumor clone contains three proviruses (named according to chromosome inserted into), the provirus on chr1 inserted into a repetitive element (LTR) and short reads generated from host DNA flanking the insertion site map to multiple positions in the genome. Filtering out multi-mapping reads causes an underestimation of the abundance of this insertion site (13.6%), this can be partially corrected by retaining multi-mapping reads at this position (25.4%). However, that approach can cause the potentially spurious inflation of other integration sites (red slice 9%). The long PCIP-seq reads can span repetitive elements and produce even coverage for each provirus without correction. (FIG. 2E) Screen shot from IGV shows representative reads coming from the three proviruses at positions where four de novo mutations were observed.

FIGS. 3A and 3B. (FIG. 3A) Screen shot from IGV shows representative reads from a subset of the clones from each BLV-infected animal with a mutation in the first base of codon 303 in the viral protein Tax. (FIG. 3B) Structural variants observed in the BLV provirus. BLV sense and antisense transcripts are shown on top. Deletions (blue bars) and duplications (red bars) observed in the BLV provirus from both ovine and bovine samples are shown below.

FIGS. 4A-4C. HPV ‘looping’ integration in an expanded clone (FIG. 4A) PCIP-seq reads mapping to a ˜87 kb region on chr3 revealed three HPV-host breakpoints. The large number of reads suggests expansion of the clone carrying these integrations. (FIG. 4B) PCR was carried out with primer pairs matching regions a and 3, as well as a and γ. Both primer pairs produced a ˜9 kb PCR product. Nanopore sequencing of the PCR products show the HPV genome connects these breakpoints. (FIG. 4C) Schematic of the breakpoints with the integrated HPV genome. This conformation indicates that this dramatic structural rearrangement in the host genome was generated via ‘looping’ integration of the HPV genome.

FIGS. 5A and 5B. (FIG. 5A) Reads from four HPV16 samples mapped to the HPV16 subtype A1 genome. Vertical lines identify position where the base differs from the reference genome. (FIG. 5B) Consensus sequences were generated for 12 HPV16 samples and a phylogenetic tree with the HPV16 subtype reference genomes (highlighted) was generated. The 12 samples cluster with the HPV16 A1 and A2, both are European isolates.

FIGS. 6A-6D. Clone persistence was observed in two patients. The first patient had an integration in the LAPTM4B gene (histology=ASC-H), a second sampling from 7 months later (upgraded to HSIL) showed the same integration sites (FIG. 6A) The discordant breakpoints again points to ‘looping’ integration in an expanded clone. (FIG. 6B) When the reads are mapped to the HPV genome the sample from July 2019 has reads originating from episomal copies of HPV as well as reads from the integrated copy of HPV. All the HPV reads from the December 2019 sample contain the deletion associated with the integrated copy of HPV indicating that the infection has cleared but the clonally expanded cell remains. PCR with primer pairs matching regions a and 13 produced a ˜9 kb PCR product, again indicating that the integration has caused a structural rearrangement in this region. (FIG. 6C) In the second patient (a 71 year old, histology=ASC-US at both time points) HPV was found to be integrated at three positions in the genome (within exons of the genes TMEM177, IL20RB and ARMH3), introducing at least three copies of HPV (E6 and E7 are intact in the p integrated HPV genomes). It is not possible to tell at this point if all are in the same or separate clones. (FIG. 6D) For both time points the integrated HPV reads represent −10% of the total HPV reads, although the greater number of unique shear sites in the second time point (especially for the chr2 integration) suggest the clone may be expanding.

FIG. 7. Use of Cas-9 mediated cleavage in the PCIP-seq method. 8 μg of DNA from a BLV infected sheep with a proviral load of 82.6% was circularized and linear DNA was eliminated. One quarter of the resultant DNA was subject to CRISPR-cas9 cleavage using the Pool A guides (CRISPR+, PA), the second quarter was cleaved using the Pool B guides (CRISPR+, PB), the remaining half was kept aside. The linearized DNA was cleaned and used as template in 2×50 μl PCR reactions using the appropriate primer pairs for Pool A (PA) or Pool B (PB). For the uncut DNA half was used as template for 2×50 μl PCR reactions using the PA primers (CRISPR−, PA) and the other half was used for 2×50 μl PCR reactions using the PB primers (CRISPR−, PB). Following 25 PCR cycles, 10 μl of each reaction were loaded on a 1% agarose gel. A=unshared genomic DNA, B=genomic DNA sheared to 8 kb.

FIGS. 8A and 8B. Coverage of the pure viral reads as well as the chimeric reads produced by the libraries shown in FIG. 7 on the BLV proviral genome. BC refers to the barcode used for each library.

FIG. 9. Pie charts showing the relative abundance of the 200 largest clones in the four sheep (top) and three cattle (bottom) infected with BLV, each slice of the pie represents a single insertion site, the % below indicated what fraction of the overall reads these 200 clones represent.

### DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any or etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

For general methods relating to the invention, reference is made inter alia to well-known textbooks, including, e.g., “Molecular Cloning: A Laboratory Manual, 4th Ed.” (Green and Sambrook, 2012, Cold Spring Harbor Laboratory Press), “Current Protocols in Molecular Biology” (Ausubel et al., 1987).

Provided herein is a method for detecting an integration pattern of a virus in genomic DNA of a subject, said method comprising:

(a) fragmenting genomic DNA isolated from a sample of the subject;

(b) circularizing the DNA fragments to generate circular DNA;

(c) removing non-circularized DNA fragments;

(d) optionally linearizing the circular DNA using an RNA-guided DNA endonuclease and at least one guide RNA or at least one pool of guide RNAs, which target a region in the viral genome to generate linearized DNA molecules;

(e) amplifying the circular DNA or the linearized DNA molecules by an inverse amplification reaction using a pair of primers arranged about and oriented outwardly with respect to the linearization site;

(f) sequencing the amplified DNA;

(g) mapping the sequenced DNA to genomic DNA sequence of the subject; and

(h) optionally mapping the sequenced DNA to the viral genome.

As used herein, the terms “integration pattern” or “viral integration pattern” refer to the pattern of viral DNA that is integrated in host genomic DNA. The term may refer to a visualized DNA pattern comprising viral DNA and host genomic DNA, as well as to information quantified by or correlated with such DNA pattern. Non-limiting examples of information quantified by, or correlated with an integration pattern include the presence of absence of integrated viral DNA; the number of viral integration sites in host genomic DNA or the average number of such integrations; the insertion site(s) of viral DNA in the host genome; mutations (e.g. deletions, duplications, SNPs, etc.) in the viral DNA integrations; the size in kb of viral DNA integrations into host genomic DNA; the number of viral genomes integrated at each integration site; the number of viral integration sites per cellular genome; the mean number of viral genomes integrated per integration site (or the mean size of integration sites); maximum number of viral genomes integrated per integration site (or the maximum size of integration sites); minimum number of viral genomes integrated per integration site (or minimum size of integration sites), number of viral genomes integrated per cellular genome, and any combinations thereof.

The method of the invention allows to detect integration of viruses such as retroviruses that integrate into a host cell genome as part of their lifecycle, as well as viruses such as papillomaviruses that do not integrate into a host cell genome as part of their lifecycle. The virus may be a DNA virus or an RNA virus. DNA viruses include, for example, human papillomavirus (HPV); RNA viruses include, for example, human T lymphophilic virus (HTLV, particularly HTLV-1), human immunodeficiency virus (HIV), bovine leukemia virus (BLV). In embodiments, the virus is a retrovirus. In further embodiments, the retrovirus is an exogenous retrovirus such as HTLV, in particular HTLV-1, HIV or BLV. In further embodiments, the retrovirus is an endogenous retrovirus. In other embodiments, the virus is HPV. In further embodiments, said HPV is a high risk HPV such as a HPV strain 16, 18, 31, 33, 35, 39, 45, 51, 55, 56, 58, 59 or 66, preferably a HPV strain 18 or a HPV strain 16.

“Integrated viral DNA” refers to a complete or partial genome of a virus that is integrated into a host cell chromosome. “Episomal viral DNA” refers to non-integrated viral DNA, i.e., viral DNA that has not integrated into a host cell chromosome. “Provirus” refers to viral DNA, in particular retroviral DNA, that is integrated into the DNA of a host cell as a stage of virus replication, or a state that persists over longer periods of time as either inactive viral infections or an endogenous viral element.

The terms “subject” and “host” and “patient” are used interchangeably and refer to a human or non-human animal that is tested for the presence of integrated viral DNA. The host is not particularly limited as long as the virus infects and viral nucleic acid is integrated into the genome. Preferably, the host is a mammal, most preferably a human. Hosts may be domestic animals such as cows, horses, pigs, sheep, goats and chickens. In preferred embodiments, the subject is a human. In embodiments, the subject is an ovine. In embodiments, the subject is a bovine.

The term “sample” generally refers to a material of biological origin that includes cells. Samples can include, e.g., an in vitro cell culture or tissue obtained from a subject as defined herein. Samples can be purified or semi-purified to remove certain constituents (e.g., extracellular constituents or non-target cell populations). In embodiments, the sample comprises cervical or vaginal epithelial cells, such as wherein the sample is a pap smear. In embodiments, the sample comprises oropharyngeal epithelial cells, such as wherein the sample is an oropharyngeal swab. In embodiments, the sample comprises peripheral blood mononuclear cells (PBMC), in particular CD4+ T cells, such as wherein the sample is a blood sample, e.g. a whole blood sample. In embodiments, the sample is a sperm sample. Isolation of DNA from the samples can be carried out by standard methods.

In step (a) genomic DNA of the subject is fragmented. In embodiments, fragmenting the genomic DNA of the subject comprises shearing the genomic DNA, thereby producing (sheared) DNA fragments. Shearing of the genomic DNA may occur e.g. by acoustic or mechanical means as known to the skilled person. In further embodiments, shearing of the genomic DNA of the subject is followed by end-repair of the sheared DNA fragments.

In embodiments, the (sheared) DNA fragments have an average size of about the size of the viral genome. In particular embodiments, the (sheared) DNA fragments have an average size of between 6000 and 10000 basepairs (bp), preferably between 7000 and 9000 bp, more preferably about 8000 bp.

In step (b) of the method (sheared) DNA fragments are circularized. Circularization or intramolecular ligation of the DNA fragments may be achieved by incubation of the DNA fragments in the presence of a DNA ligase, e.g. T4 DNA ligase, as known to the skilled person, thereby generating circular DNA.

Step (c) of the method encompasses removal of remaining linear DNA. In embodiments, non-circularized DNA is removed by digestion. Selective digestion of non-circularized or linear DNA may be achieved using an appropriate selective DNase as commercially available (e.g. Plasmid-Safe™ ATP-Dependent DNase (Epicentre).

Preferably, the circular DNA is linearized in step (d) before the amplification step (e), which improves the efficiency of the amplification reactions. Linearization of the circular DNA can be achieved using an RNA-guided DNA endonuclease, such as a CRISPR-Cas system as known to the skilled person, and corresponding guide RNAs. In particular embodiments, the RNA-guided DNA endonuclease is a Cas-9 endonuclease.

In order to achieve selective linearization of circular DNA that comprises integrated viral DNA and host DNA, guide RNA(s) are used that target a region of the viral DNA. Preferably, the “linearization site”, i.e. the region in the viral DNA that is targeted by a guide RNA or a pool of guide RNAs, comprises a region of the viral genome that is prone to integration in host DNA. For example, for HPV, a linearization site may comprise E6 gene and/or E7 gene. For retroviruses, a linearization site may be adjacent to a 5′LTR or adjacent to a 3′LTR.

Particular guide RNA targeting domains and pools of guide RNA targeting domains are provided in Table 1. The sequences set forth in SEQ ID NO:7-79 refer to oligonucleotide sequences used for synthesizing the guide RNAs. These sequences comprise a “targeting domain” as well as accessory sequences required by the kit, in particular the EnGen® sgRNA Synthesis Kit (New England Biolabs), for synthesizing the guide RNA, which elements can be identified by the skilled person. By way of example, oligonucleotide sequences encoding HPV18 and HPV16 gRNAs and their corresponding targeting domain and flanking PAM site (underlined) are summarized in the below table. With “targeting domain” is meant herein a sequence that is capable of hybridizing to a sequence in the region of the viral DNA that is targeted by the guide RNA (i.e. in the linearization site of the viral DNA). With “PAM site” is meant herein a protospacer adjacent sequence as is known in the art. When reference is made to a guide RNA comprising a sequence set forth in any one of SEQ ID NO:7-79, a guide RNA comprising the targeting domain of said sequence is envisaged, i.e. the sequence without the sequence TTCTAATACGACTCACTATA (SEQ ID NO:244) 5 prime and without the sequence GTTTTAGAGCTAGA (SEQ ID NO:245) 3 prime. When reference is made to a guide RNA comprising a sequence set forth in any one of SEQ ID NO:232-243, a guide RNA comprising the targeting of said sequence is envisaged, i.e. the sequence without the NGG sequence 3 prime. As will be appreciated by the skilled person, the guide RNA comprises in addition to a targeting domain, a tracer and a tracer mate as known in the art, wherein the tracer and tracer mate may be provided chimeric. The guide RNA is an RNA molecule and will therefore comprise the base uracil (U), while the oligonucleotide encoding the gRNA molecule comprises the base thymine (T).

| | | | Targeting | |
|---|---|---|---|---|
| | | SEQ | domain and | SEQ |
| | | ID | flanking PAM | ID |
| Guide RNA | Oligonucleotide | NO: | site | NO: |
| HPV18 Region 1 | | | | |
| Guide RNA | | | | |
| 1_H_PV18_R1_ | TTCTAATACGACTCACTATAGTGCTGCA | 68 | GTGCTGCAACCG | 232 |
| guide1 | ACCGAGCACGACGTTTTAGAGCTAGA | | AGCACGACAGG | |
| 2_HPV18_R1_ | TTCTAATACGACTCACTATAGTGCTCGG | 69 | GTGCTCGGTTGC | 233 |
| guide2 | TTGCAGCACGAAGTTTTAGAGCTAGA | | AGCACGAATGG | |
| 3_H_PV18_R1_ | TTCTAATACGACTCACTATAGCGACGAT | 70 | CGACGATTTCAC | 234 |
| guide3 | TTCACAACATAGCGTTTTAGAGCTAGA | | AACATAGCTGG | |
| HPV18_Region_2 | | | | |
| Guide RNA | | | | |
| 8_HPV18_R2_ | TTCTAATACGACTCACTATAGATTTTAG | 71 | ATTTTAGAGGAT | 235 |
| guide4 | AGGATTGGAACTTGTTTTAGAGCTAGA | | TGGAACTTTGG | |
| 9_HPV18_R2_ | TTCTAATACGACTCACTATAGTCTGCTA | 72 | TCTGCTATACTG | 236 |
| guide5 | TACTGCTTAAATTGTTTTAGAGCTAGA | | CTTAAATTTGG | |
| 10_HPV18_R2_ | TTCTAATACGACTCACTATAGCATCATA | 73 | GCATCATATTGC | 237 |
| guide6 | TTGCCCAGGTACGTTTTAGAGCTAGA | | CCAGGTACAGG | |
| HPV16_E6-E7 | | | | |
| Guide RNA | | | | |
| 3261_HPV16_E6- | TTCTAATACGACTCACTATAGCTAATTA | 74 | CTAATTAACAAA | 238 |
| E7_G1 | ACAAATCACACAAGTTTTAGAGCTAGA | | TCACACAACGG | |
| 3262_HPV16_E6- | TTCTAATACGACTCACTATAGATTCCAT | 75 | GATTCCATAATA | 239 |
| E7_G2 | AATATAAGGGGTGTTTTAGAGCTAGA | | TAAGGGGTCGG | |
| 3263_HPV16_E6- | TTCTAATACGACTCACTATAGCAACAAG | 76 | GCAACAAGACAT | 240 |
| E7_G3 | ACATACATCGACGTTTTAGAGCTAGA | | ACATCGACCGG | |
| HPV16_L1 | | | | |
| Guide RNA | | | | |
| 3266_HPV16_L1_G1 | TTCTAATACGACTCACTATAGCCACCTA | 77 | CCACCTATAGGG | 241 |
| | TAGGGGAACACTGGTTTTAGAGCTAGA | | GAACACTGGGG | |
| 3267_HPV16_L1_G2 | TTCTAATACGACTCACTATAGACCTACC | 78 | ACCTACCTCAAC | 242 |
| | TCAACACCTACACGTTTTAGAGCTAGA | | ACCTACACAGG | |
| 3268_HPV16_L1_G3 | TTCTAATACGACTCACTATAGTAATAGA | 79 | TAATAGAGAATG | 243 |
| | GAATGTATATCTAGTTTTAGAGCTAGA | | TATATCTATGG | |

To improve cleavage of a linearization site, more than one guide RNA targeting said linearization site can be used. As used herein, a “pool of guide RNAs” refers to a set of guide RNAs that target a defined region of the viral DNA, i.e. the linearization site. It is to be understood that each guide RNA within a pool of guide RNAs may be capable of hybridizing to different, non-overlapping or partially overlapping, sequences within said linearization site. A pool of guide RNAs may comprise at least 2 or at least 3 guide RNAs, preferably at least 3 guide RNAs, more preferably between 3 and 10 or between 3 and 8 guide RNAs, such as 3, 4, 5, 6, 7 or 8 guide RNAs.

The circular DNA may be linearized using a first guide RNA or a first pool of guide RNAs, which target a first region of the viral DNA, and at least one other guide RNA or at least one other pool of guide RNAs, which target a non-overlapping region(s) of the viral RNA. When targeting more than one linearization site, a more complete integration pattern may be obtained (e.g. more integration sites may be detected).

Accordingly, in embodiments, a first portion of the circular DNA is linearized using a first guide RNA or a first pool of guide RNAs that target a first region of the viral DNA to generate a first set of linearized DNA molecules; and

a second portion of the circular DNA is linearized using a second guide RNA or a second pool of guide RNAs that target a second region of the viral DNA to generate a second set of linearized DNA molecules,

wherein the first region and the second region of the viral DNA do not overlap.

In embodiments of the method for detecting an integration pattern of a retrovirus in genomic DNA of a subject, a first portion of the circular DNA is linearized using a first guide RNA or a first pool of guide RNAs that target a region of the viral DNA adjacent to the 5′ long terminal repeat (LTR) to generate a first set of linearized DNA molecules; and

a second portion of the circular DNA is linearized using a second guide RNA or a second pool of guide RNAs that target a region of the viral DNA adjacent to the 3′LTR to generate a second set of linearized DNA molecules.

In embodiments of the method for detecting an integration pattern of a HPV in genomic DNA of a subject, a first portion of the circular DNA is linearized using a first guide RNA or a first pool of guide RNAs that target a first region of the viral DNA comprising E6 gene and/or E7 gene to generate a first set of linearized DNA molecules; and

a second portion of the circular DNA is linearized using a second guide RNA or a second pool of guide RNAs that target a second region of the viral DNA to generate a second set of linearized DNA molecules, wherein said first and second regions of the viral DNA do not overlap.

In the amplification step (e), the circular DNA or preferably the linearized DNA molecules are amplified by an inverse amplification reaction using a pair of primers arranged about and oriented outwardly with respect to the linearization site. In particular, a primer pair is used comprising a forward primer capable of hybridizing to a viral DNA sequence in a 3′ flanking region of the viral DNA region targeted by the guide RNA or the pool of guide RNAs and a reverse primer capable of hybridizing to a viral DNA sequence in a 5′ flanking region of the viral DNA region targeted by the guide RNA or the pool of guide RNAs.

Particular primer pairs corresponding to the guide RNA targeting domains or pools of guide RNA targeting domains of Table 1 are provided in Table 2. The primers in Table 2 may comprise a tail, in particular a tail consisting of the sequence TTTCTGTTGGTGCTGATATTGC (SEQ ID NO:246) or the sequence ACTTGCCTGTCGCTCTATCTTC (SEQ ID NO:247). When reference is made herein to a primer comprising a sequence set forth in any one of SEQ ID NO:80-127, the tailed primer as well as a corresponding primer without the tail or with another tail are envisaged herein.

Preferably, each set of linearized DNA molecules (i.e. linearized DNA molecules generated by one guide RNA or one pool of guide RNAs as described herein and thus characterized by cleavage in a defined linearization site) is amplified in a separate amplification reaction using an appropriate pair of primers arranged about and oriented outwardly with respect to the linearization site.

In further embodiments, the linearization step and the amplification step may be carried out in a single solution, wherein a guide RNA or a pool of guide RNAs and a corresponding pair of primers are multiplexed.

In preferred embodiments, said amplification reaction comprises a long range amplification reaction such as a long range PCR. As used herein, “long range PCR” refers to a method to amplify DNA fragments of increased size, typically of more than 3-5 kb, using a modified DNA polymerase or high-fidelity DNA polymerase. DNA polymerases for long range PCR are known to the skilled person and are commercially available.

In further embodiments, tailed primers are used in the amplification reaction and the amplicons are subjected to a second amplification reaction using a set of indexing primers, thereby generating indexed amplification products. This facilitates multiplexed sequencing of the amplified DNA.

Particular methods are provided herein for detecting an integration pattern of a retrovirus in genomic DNA of a subject, said method comprising:

(a) fragmenting genomic DNA isolated from a sample of the subject;

(b) circularizing the DNA fragments to generate circular DNA;

(c) removing non-circularized DNA fragments;

(d) linearizing the circular DNA using an RNA-guided DNA endonuclease and at least one guide RNA or at least one pool of guide RNAs, which target a region in the viral genome adjacent to the 5′ long terminal repeat (LTR) or adjacent to the 3′LTR to generate linearized DNA molecules;

(e) amplifying the linearized DNA molecules by an inverse amplification reaction using a pair of primers arranged about and oriented outwardly with respect to the linearization site;

(f) sequencing the amplified DNA;

(g) mapping the sequenced DNA to genomic DNA sequence of the subject; and

(h) optionally mapping the sequenced DNA to the viral genome.

In further embodiments of the method for detecting an integration pattern of a retrovirus in genomic DNA of a subject, the linearization of the circular DNA comprises linearizing a first portion of the circular DNA using a first guide RNA or a first pool of guide RNAs, preferably a first pool of guide RNAs, which target a region of the viral DNA adjacent to the 5′ long terminal repeat (LTR) to generate a first set of linearized DNA molecules, and

linearizing a second portion of the circular DNA using a second guide RNA or a second pool of guide RNAs, preferably a second pool of guide RNAs, which target a region of the viral DNA adjacent to the 3′LTR to generate a second set of linearized DNA molecules; and

the amplification of the linearized DNA molecules comprises amplifying the first set of linearized DNA molecules using a first pair of primers arranged about and oriented outwardly with respect to the viral DNA region adjacent to the 5′ LTR targeted by the first guide RNA or the first pool of guide RNAs,

and amplifying the second set of linearized DNA molecules using a second pair of primers arranged about and oriented outwardly with respect to the viral DNA region adjacent to the 3′ LTR targeted by the second guide RNA or the second pool of guide RNAs.

A further aspect relates to a kit for performing the method described herein, said kit comprising:
at least one first guide RNA or at least one first pool of guide RNAs, which target a first region of the viral DNA; and/or, preferably and,
a pair of primers arranged about and oriented outwardly with respect to a first linearization site in the viral DNA defined by said at least one first guide RNA or at least one first pool of guide RNAs.

In further embodiments, the kit comprises:
a first guide RNA or a first pool of guide RNAs, which target a first region of the viral DNA;
a second guide RNA or a second pool of guide RNAs, which target a second region of the viral DNA, wherein the first and the second regions of the viral DNA do not overlap;
a first pair of primers arranged about and oriented outwardly with respect to a first linearization site in the viral DNA defined by said first guide RNA or said first pool of guide RNAs; and/or, preferably and,

a second pair of primers arranged about and oriented outwardly with respect to a second linearization site in the viral DNA defined by said second guide RNA or said second pool of guide RNAs.

Particular kits are provided herein for the detection of an integration pattern of a HPV in genomic DNA of a subject according to the method disclosed herein, said kit comprising:
at least one guide RNA or at least one pool of guide RNAs, which target a region of the viral DNA comprising E6 gene and/or E7 gene; and/or, preferably and
a pair of primers arranged about and oriented outwardly with respect to a linearization site in the viral DNA defined by said at least one guide RNA or at least one pool of guide RNAs.

In other embodiments, said kit comprises:
at least one guide RNA or at least one pool of guide RNAs, which target a region of the viral DNA comprising or adjacent to L1 gene; and
a pair of primers arranged about and oriented outwardly with respect to a linearization site in the viral DNA defined by said at least one guide RNA or at least one pool of guide RNAs.

In further embodiments, said kit for the detection of an integration pattern of a HPV comprises:
a first guide RNA or a first pool of guide RNAs, which target a first region of the viral DNA comprising E6 gene and/or E7 gene;
a first pair of primers arranged about and oriented outwardly with respect to a linearization site in the viral DNA defined by said first guide RNA or said first pool of guide RNAs;
a second guide RNA or a second pool of guide RNAs, which target a second region of the viral DNA, wherein said first and second regions of the viral DNA do not overlap; and
a second pair of primers arranged about and oriented outwardly with respect to a linearization site in the viral DNA defined by said second guide RNA or said second pool of guide RNAs.

In particular embodiments, said second region of the viral DNA comprises a region of the viral DNA comprising L1 gene or a region of the viral DNA adjacent to L1 gene.

Particular embodiments for the guide RNAs, pools of guide RNAs and primer pairs are as described above for the method. Particular combinations of guide RNA targetind domains or pools of guide RNA targeting domains and primer pairs are described in Tables 1 and 2.

The kit may also contain reagents, e.g., buffers, enzymes and other necessary reagents, for performing the method described above. In particular embodiments, the kit further comprises an RNA-guided DNA endonuclease. In particular embodiments, the kit further comprises a DNA polymerase, preferably a DNA polymerase for long range PCR.

The various components of the kit may be present in separate containers or certain compatible components may be pre-combined into a single container, as desired.

The herein disclosed aspects and embodiments of the invention are further supported by the following non-limiting examples.

### EXAMPLES

### Example 1: Materials and Methods

Samples

Both the BLV infected sheep⁷ and HTLV-1 samples^{7,20} have been previously described. Briefly, the sheep were infected with the molecular clone pBLV344²¹, following the experimental procedures approved by the University of Saskatchewan Animal Care Committee based on the Canadian Council on Animal Care Guidelines (Protocol #19940212). The HTLV-1 samples^{7,20} were obtained with informed consent following the institutional review board-approved protocol at the Necker Hospital, University of Paris, France, in accordance with the Declaration of Helsinki. The BLV bovine samples were natural infections, obtained from commercially kept adult dairy cows in Alberta, Canada. Sampling was approved by VSACC (Veterinary Sciences Animal care Committee) of the University of Calgary: protocol number: AC15-0159. The bovine 571 used for ERV identification was collected as part of this cohort. The two sheep samples used for Jaagsiekte sheep retrovirus (enJSRV) identification were the BLV infected ovine samples (220 & 221 (032014)), with a PVL of 3.8 and 16% respectively. PBMCs were isolated using standard Ficoll-Hypaque separation. The DNA for the bovine Mannequin was extracted from sperm, while the DNA for bovine 10201e6 was extracted from whole blood using standard procedures. The HIV-1 U1 cell line DNA sequenced without dilution was provided by Dr. Carine Van Lint, IBMM, Gosselies, Belgium. The HIV-1 U1 cell line dilutions in Jurkat were generated at Ghent University Hospital.

HPV material was prepared from PAP smears obtained from HPV-infected patients at the CHU Liege University hospital. Both patients were PCR positive for HPV18, HPV18_PY was classified as having Atypical Squamous Cell of Undetermined Significance (ASC-US), while HPV18_PX was classified as having Atypical Glandular Cells (AGC). Patients provided written informed consent and the study was approved by the Comité d'Ethique Hospitalo-Facultaire Universitaire de Liege (Reference number: 2019/139). No statistical test was used to determine adequate sample size and the study did not use blinding.

PCIP-Seq

Total genomic DNA isolation was carried out using the Qiagen AllPrep DNA/RNA/miRNA kit (BLV, HTLV-1 and HPV infected individuals) or the Qiagen DNeasy Blood & Tissue Kit (HIV-1 patients) according to manufacturer's protocol. High molecular weight DNA was sheared to ˜8 kb using Covaris g-Tubes™ (Woburn, Mass.) or a Megaruptor (Diagenode), followed by end-repair using the NEBNext EndRepair Module (New England Biolabs). Intramolecular circularization was achieved by overnight incubation at 16° C. with T4 DNA Ligase. Remaining linear DNA was removed with Plasmid-Safe-ATP-Dependent DNAse (Epicentre, Madison Wis.). Guide RNAs were designed using chopchop (http://chopchop.cbu.uib.no/index.php). The EnGen™ sgRNA Template Oligo Designer (http://nebiocalculator.neb.com/#!/sgrna) provided the final oligo sequence. Oligos were synthesized by Integrated DNA Technologies (IDT). Oligos were pooled and guide RNAs synthesized with the EnGen sgRNA Synthesis kit, S. pyogenes (New England Biolabs). Selective linearization reactions were performed with the Cas-9 nuclease, S. pyogenes (New England Biolabs). (See Example 3 for the rationale behind using of CRISPR-cas9 to cleave the circular DNA). PCR primers flanking the cut sites were designed using primer3 (http://bioinfo.ut.ee/primer3/). Primers were tailed to facilitate the addition of Oxford Nanopore indexes in a subsequent PCR reaction. The linearized fragments were PCR amplified with LongAmp Taq DNA Polymerase (New England Biolabs) and purified using 1× AmpureXP beads, (Beckman Coulter). A second PCR added the appropriate Oxford Nanopore index. PCR products were visualized on a 1% agarose gel, purified using 1× AmpureXP beads and quantified on a Nanodrop spectrophotometer. Indexed PCR products were multiplexed and Oxford Nanopore libraries prepared with either the Ligation Sequencing Kit 1D (SQK-LSK108) or 1D\hat{ }2 Sequencing Kit (SQK-LSK308) (only the 1D were used) The resulting libraries were sequenced on Oxford Nanopore MinION R9.4 or R9.5 flow cells respectively. The endogenous retrovirus libraries were base called using albacore 2.3.1, all other PCIP-seq libraries were base called with Guppy 3.1.5 (https://nanoporetech.com) using the “high accuracy” base calling model. For the endogenous retrovirus libraries, demultiplexing was carried out via porechop (https://github.com/rrwick/Porechop) using the default setting. The HIV, HTLV-1, BLV and HPV PCIP-seq libraries were subjected to a more stringent demultiplexing with the guppy_barcoder (https://nanoporetech.com) tool using the --require_barcodes_both_ends option. The output was also passed through porechop, again barcodes were required on both ends, adapter sequence was trimmed and reads with middle adapters were discarded. Oligos used can be found in Tables 1 and 2.

TABLE 1
Guide RNA oligo's.

| | | SEQ |
|---|---|---|
| | | ID |
| Guide Pool | Guide RNA Oligos | NO |
| BLV-Pool-A | | |
| (used in Bov & OAR) | | |
| 2563-BLV-Guide31_5PA | TTCTAATACGACTCACTATAGTCTGAGGGGGAGATACCAGCGTTTTAGAG | 7 |
| | CTAGA | |
| 2564-BLV-Guide32_5PA | TTCTAATACGACTCACTATAGAAGACCCAAAACGCCGCCGAGTTTTAGAG | 8 |
| | CTAGA | |
| 2565-BLV-Guide33_5PA | TTCTAATACGACTCACTATAGCACCCCCTCGGCGGCGTTTTGTTTTAGAG | 9 |
| | CTAGA | |
| 2597-BLV-Guide43_3PA | TTCTAATACGACTCACTATAGACAGCCGGAGGGGGTCCACAGTTTTAGAG | 10 |
| | CTAGA | |
| 2598-BLV-Guide44_3PA | TTCTAATACGACTCACTATAGTTAGTAACGCATCCTGTCCTGTTTTAGAG | 11 |
| | CTAGA | |
| 2599-BLV-Guide45_3PA | TTCTAATACGACTCACTATAGCCCTCCTTGTGGACCCCCTCGTTTTAGAG | 12 |
| | CTAGA | |
| 2560-BLV-Guide46_3PA | TTCTAATACGACTCACTATAGCAAAGACGGACAGCCGGAGGGTTTTAGAG | 13 |
| | CTAGA | |
| BLV Pool B | | |
| (used in OAR) | | |
| 2570-BLV-Guide34_5PB | TTCTAATACGACTCACTATAGCTTCTGGGGCCGATGCACCCGTTTTAGAG | 14 |
| | CTAGA | |
| 257l-BLV-Guide35_5PB | TTCTAATACGACTCACTATAGCGAAGTGCTCTCAAACGATGGTTTTAGAG | 15 |
| | CTAGA | |
| 2572-BLV-Guide36_5PB | TTCTAATACGACTCACTATAGAACGGCGGGGGGGTCATAAGGTTTTAGAG | 16 |
| | CTAGA | |
| 2584-BLV-Guide40_3PB | TTCTAATACGACTCACTATAGGTTAGGAATAGGTCGATCGGTTTTAGAGC | 17 |
| | TAGA | |
| 2585-BLV-Guide41_3PB | TTCTAATACGACTCACTATAGTAACCGGTCGCATGGGGAAGGTTTTAGAG | 18 |
| | CTAGA | |
| 2586-BLV-Guide42_3PB | TTCTAATACGACTCACTATAGAGGAAGCGTTGTAAGGCCTGGTTTTAGAG | 19 |
| | CTAGA | |
| BLV BOV Pool B | | |
| (used in OAR) | | |
| 2570-BLV-Guide34_5PB | TTCTAATACGACTCACTATAGCTTCTGGGGCCGATGCACCCGTTTTAGAG | 20 |
| | CTAGA | |
| 257l-BLV-Guide35_5PB | TTCTAATACGACTCACTATAGCGAAGTGCTCTCAAACGATGGTTTTAGAG | 21 |
| | CTAGA | |
| 2572-BLV-Guide36_5PB | TTCTAATACGACTCACTATAGAACGGCGGGGGGGTCATAAGGTTTTAGAG | 22 |
| | CTAGA | |
| 2584-BLV-Guide40_3PB | TTCTAATACGACTCACTATAGGTTAGGAATAGGTCGATCGGTTTTAGAGC | 23 |
| | TAGA | |
| 2585-BLV-Guide41_3PB | TTCTAATACGACTCACTATAGTAACCGGTCGCATGGGGAAGGTTTTAGAG | 24 |
| | CTAGA | |
| 2691-BLV-Guide48_3PB | TTCTAATACGACTCACTATAGCTGCCCCTTATCCAAACGCCGTTTTAGAG | 25 |
| | CTAGA | |
| BosT ERV Pool A | | |
| 2652-BosT_ERV_G7-PB5 | TTCTAATACGACTCACTATAGAGGTTGTTCCTGAGTAGTCAGTTTTAGAG | 26 |
| | CTAGA | |
| 2663-BosT_ERV_G8-PB5 | TTCTAATACGACTCACTATAGTGTTCCTCATCCCTATCTTTGTTTTAGAG | 27 |
| | CTAGA | |
| 2664-BosT_ERV_G9-PB5 | TTCTAATACGACTCACTATAGACAACTAAATATCACTCTGAGTTTTAGAG | 28 |
| | CTAGA | |
| BosT ERV Pool B | | |
| 2657-BosT_ERV_G10-PC3 | TTCTAATACGACTCACTATAGCAAGGTAGCGTAGCCGAGGAGTTTTAGAG | 29 |
| | CTAGA | |
| 2658-BosT_ERV_Gll-PC3 | TTCTAATACGACTCACTATAGAAATCATTTGCTGTTCCAGGTTTTAGAGC | 30 |
| | TAGA | |
| 2659-BosT_ERV_Gll-PC3 | TTCTAATACGACTCACTATAGGGGTGTTACACATATCCACGTTTTAGAGC | 31 |
| | TAGA | |
| Oar JSRV Pool A | | |
| 2627-JSRV_G9-5PA | TTCTAATACGACTCACTATAGTCGAGACCAGCCACAACAGAGTTTTAGAG | 32 |
| | CTAGA | |
| 2628-JSRV_G10-5PA | TTCTAATACGACTCACTATAGGGTTGCTTTCAACCCCCTCGTTTTAGAGC | 33 |
| | TAGA | |
| 2629-JSRV_Gll-5PA | TTCTAATACGACTCACTATAGACTATTGCTTTACAGAACGCGTTTTAGAG | 34 |
| | CTAGA | |
| 2642-JSRV_G18-3PA | TTCTAATACGACTCACTATAGTTACAGCGGATACAAAACGGTTTTAGAGC | 35 |
| | TAGA | |
| 2643-JSRV_G19-3PA | TTCTAATACGACTCACTATAGAAGGCTGGTACGCGCGGCAGGTTTTAGAG | 36 |
| | CTAGA | |
| 2644-JSRV_G20-3PA | TTCTAATACGACTCACTATAGATGTCGAGCACGAATTGCATGTTTTAGAG | 37 |
| | CTAGA | |
| Oar JSRV Pool B | | |
| 2632-JSRV_G12-5PB | TTCTAATACGACTCACTATAGATCTTTCAAAAGTCCGGCAGTTTTAGAGC | 38 |
| | TAGA | |
| 2633-JSRV_G13-5PB | TTCTAATACGACTCACTATAGCTGATGTTAACCGACAGCAGTTTTAGAGC | 39 |
| | TAGA | |
| 2634-JSRV G14-5PB | TTCTAATACGACTCACTATAGCACAAATATCAAATGCGGCTGTTTTAGAG | 40 |
| | CTAGA | |
| 2637-JSRV_G15-3PB | TTCTAATACGACTCACTATAGGCTCAGACCTCTTTTAGGAGTTTTAGAGC | 41 |
| | TAGA | |
| 2638-JSRV_G16-3PB | TTCTAATACGACTCACTATAGTTCTGACTTTCCGTGGGATAGTTTTAGAG | 42 |
| | CTAGA | |
| 2639-JSRV_G17-3PB | TTCTAATACGACTCACTATAGATTTTGTAAAAAATTATCGAGTTTTAGAG | 43 |
| | CTAGA | |
| HTLV1 Pool A | | |
| 2604-HTLV1_G21-5PA | TTCTAATACGACTCACTATAGCTGGTGGAAATCGTAACTGGGTTTTAGAG | 44 |
| | CTAGA | |
| 2605-HTLV1_G22-5PA | TTCTAATACGACTCACTATAGTCCCAAAAGGATACCCCGGCGTTTTAGAG | 45 |
| | CTAGA | |
| 2606-HTLV1_G23-5PA | TTCTAATACGACTCACTATAGTAAAATTTCATTCACCCGGCGTTTTAGAG | 46 |
| | CTAGA | |
| 2611-HTLV1_G24-3PA | TTCTAATACGACTCACTATAGCGGGGTGGCAAAAAATCACGGTTTTAGAG | 47 |
| | CTAGA | |
| 2612-HTLV1_G25-3PA | TTCTAATACGACTCACTATAGGGTGTACAGGTTTTGGGGCGTTTTAGAGC | 48 |
| | TAGA | |
| 2613-HTLV1_G26-3PA | TTCTAATACGACTCACTATAGTTTGCCACCCCGGCCAGCTCGTTTTAGAG | 49 |
| | CTAGA | |
| HTLV1 Pool B | | |
| 2616-HTLV1_G27-5PB | TTCTAATACGACTCACTATAGCATGACTGGAAGGACTTGGGGTTTTAGAG | 50 |
| | CTAGA | |
| 2617-HTLV1_G28-5PB | TTCTAATACGACTCACTATAGGATGGTCTGCATAAACTGGGTTTTAGAGC | 51 |
| | TAGA | |
| 2618-HTLV1_G29-5PB | TTCTAATACGACTCACTATAGCAAACTGCTGCACCGCAAGCGTTTTAGAG | 52 |
| | CTAGA | |
| 2619-HTLV1_G3O-3PB | TTCTAATACGACTCACTATAGGAAATCATAGGCGTGCCATGTTTTAGAGC | 53 |
| | TAGA | |
| 2620-HTLV1_G31-3PB | TTCTAATACGACTCACTATAGGCTGGCCATCTTTAGGGCAGTTTTAGAGC | 54 |
| | TAGA | |
| 2621-HTLV1_G32-3PB | TTCTAATACGACTCACTATAGAGGACTGTAGTACTAAAGAGTTTTAGAGC | 55 |
| | TAGA | |
| 2622-HTLV1_G33-3PB | TTCTAATACGACTCACTATAGATGGCACGCCTATGATTTCCGTTTTAGAG | 56 |
| | CTAGA | |
| HIV UI Pool A | | |
| 2667-HIV_G1-5PA | TTCTAATACGACTCACTATAGAGAGCGTCGGTATTAAGCGGGTTTTAGAG | 57 |
| | CTAGA | |
| 2668-HIV_G2-5PA | TTCTAATACGACTCACTATAGCGGGGGAGAATTAGATAAAGTTTTAGAGC | 58 |
| | TAGA | |
| 2681-HIV_G9-3PA | TTCTAATACGACTCACTATAGAGGCGGGTCTGGAACGATAAGTTTTAGAG | 59 |
| | CTAGA | |
| 2682-HIV_G10-3PA | TTCTAATACGACTCACTATAGCACTCATCTGGGTCGATCTGGTTTTAGAG | 60 |
| | CTAGA | |
| 2683-HIV_G11-3PA | TTCTAATACGACTCACTATAGAATCCATTCACTAATGGTCGTTTTAGAGC | 61 |
| | TAGA | |
| HIV UI Pool B | | |
| 2671-HIV_G3-5PB | TTCTAATACGACTCACTATAGCATGCAGGGCCTATTGCACCGTTTTAGAG | 62 |
| | CTAGA | |
| 2672-HIV_G4-5PB | TTCTAATACGACTCACTATAGATTGCATCCAGTGCATGCAGTTTTAGAGC | 63 |
| | TAGA | |
| 2673-HIV_G5-5PB | TTCTAATACGACTCACTATAGCAATAGGCCCTGCATGCACGTTTTAGAGC | 64 |
| | TAGA | |
| 2676-HIV_G6-3PB | TTCTAATACGACTCACTATAGCAAAACGTAGTATGAGTGGAGTTTTAGAG | 65 |
| | CTAGA | |
| 2677-HIV_G7-3PB | TTCTAATACGACTCACTATAGCTACTAATGCTAATTGTGCCGTTTTAGAG | 66 |
| | CTAGA | |
| 2678-HIV_G8-3PB | TTCTAATACGACTCACTATAGCGAACTGAACCAGCAGCAGAGTTTTAGAG | 67 |
| | CTAGA | |
| HPV18 Region 1 Guide | | |
| RNA | | |
| 1_H PV18_R1_guidel | TTCTAATACGACTCACTATAGTGCTGCAACCGAGCACGACGTTTTAGAGC | 68 |
| | TAGA | |
| 2_HPV18_R1_guide2 | TTCTAATACGACTCACTATAGTGCTCGGTTGCAGCACGAAGTTTTAGAGC | 69 |
| | TAGA | |
| 3_HPV18_R1_guide3 | TTCTAATACGACTCACTATAGCGACGATTTCACAACATAGCGTTTTAGAG | 70 |
| | CTAGA | |
| HPV18 Region 2 Guide | | |
| RNA | | |
| 8_HPV18_R2_guide4 | TTCTAATACGACTCACTATAGATTTTAGAGGATTGGAACTTGTTTTAGAG | 71 |
| | CTAGA | |
| 9_HPV18_R2_guide5 | TTCTAATACGACTCACTATAGTCTGCTATACTGCTTAAATTGTTTTAGAG | 72 |
| | CTAGA | |
| 10_HPV18_R2_guide6 | TTCTAATACGACTCACTATAGCATCATATTGCCCAGGTACGTTTTAGAGC | 73 |
| | TAGA | |
| HPV16_E6-E7 Guide RNA | | |
| 3261_HPV16_E6-E7_G1 | TTCTAATACGACTCACTATAGCTAATTAACAAATCACACAAGTTTTAGAG | 74 |
| | CTAGA | |
| 3262_HPV16_E6-E7_G2 | TTCTAATACGACTCACTATAGATTCCATAATATAAGGGGTGTTTTAGAGC | 75 |
| | TAGA | |
| 3263_HPV16_E6-E7_G3 | TTCTAATACGACTCACTATAGCAACAAGACATACATCGACGTTTTAGAGC | 76 |
| | TAGA | |
| HPV16_L1 | | |
| 3266_HPV16_L1_G1 | TTCTAATACGACTCACTATAGCCACCTATAGGGGAACACTGGTTTTAGAG | 77 |
| | CTAGA | |
| 3267_HPV16_L1_G2 | TTCTAATACGACTCACTATAGACCTACCTCAACACCTACACGTTTTAGAG | 78 |
| | CTAGA | |
| 3268_HPV16_L1_G3 | TTCTAATACGACTCACTATAGTAATAGAGAATGTATATCTAGTTTTAGAG | 79 |
| | CTAGA | |

TABLE 2
Primers used for amplification of linearized DNA molecules

| | | SEQ | |
|---|---|---|---|
| | | ID | |
| | PCR primers | NO: | |
| BLV Pool A | | | |
| 2568-BLV_5PA- | TTTCTGTTGGTGCTGATATTGCGCGACCCTCTCCTA | 80 | psp344:718-739 |
| minION-E | GCGATTTT | | |
| 2595-BLV_5PA- | ACTTGcCTGTCGCTCTATCTTCGTTAGGGTTCCGGG | 81 | psp344:551-572 |
| minION-R | GTGATCAA | | |
| 2601-BLV_3PA- | TTTCTGTTGGTGCTGATATTGCCTCCACCCTTTTGA | 82 | psp344:7815-7836 |
| minION-E | CGCTATGC | | |
| 2602-BLV_3PA- | ACTTGCCTGTGGCTCTATCTTCATTGGCATTGGTAG | 83 | psp344:7585-7606 |
| minION-R | GGCTGGAA | | |
| BLV Pool B | | | |
| 2575-BLV_5PB- | TTTCTGTTGGTGCTGATATTGCCCCGCCGTTTTGCC | 84 | psp344:944-965 |
| minION-E | AATCATAT | | |
| 2576-BLV_5PB- | ACTTGCCTGTCCCTCTATCTTCTTTAGGGTGGCCAA | 85 | psp344:849-870 |
| minION-R | GACAAGGG | | |
| 2589-BLV_3PB- | TTTCTGTTGGTGCTGATATTGOTCAGAATTGGTTGC | 86 | psp344:8089-8110 |
| minION-E | TAGCGGGA | | |
| 2603-BLV_3PB- | ACTTGCCTGTCGCTCTATCTTCTTTGGATAAGGGGC | 87 | psp344:7933-7954 |
| minION-R | AGCTCGAA | | |
| BLV BOV Pool B | | | |
| 2575-BLV_5PB- | TTTCTGTTGGTGCTGATATTGCCCCGCCGTTTTGCC | 88 | psp344:944-965 |
| minION-E | AATCATAT | | |
| 2576-BLV_5PB- | ACTTCCCTGTCGCTCTATCTTCTTTAGGGTGGCCAA | 89 | psp344:849-870 |
| minION-R | GACAAGGG | | |
| 2690-BLV_3PB- | TTTCTGTTGGTGCTGATATTGCGGTCCAGTCCTCAG | 90 | psp344:8036-8056 |
| minION-E | GCCTTAC | | |
| 2603-BLV_3PB- | ACTTGCCTGTCGCTCTATCTTCTTTGGATAAGGGGC | 91 | psp344:7933-7954 |
| minION-R | AGCTCGAA | | |
| BosT ERV Pool A | | | |
| 2650- | TTTCTGTTGGTGCTGATATTGCCTGTCAGACCATCC | 92 | ChrX_ERV_denovo:23 |
| BosT_ERV_PB5-F | GCTCCTAG | | 05-2326 |
| 2651- | ACTTGCCTGTCGCTCTATCTTCTAGTCAGGCGGGTC | 93 | ChrX_ERV_denovo:20 |
| BosT_ERV_PB5-R | TTCGTTTT | | 95-2116 |
| BosT ERV Pool B | | | |
| 2655- | TTTCTGTTGGTGCTGATATTGCTCTTCGGCAGAGCA | 94 | ChrX_ERV_denovo:57 |
| BosT ERV PC3-F | TTCAGAGG | | 18-5739 |
| 2656- | ACTTGCCTGTCGCTCTATCTTCAAGTAAGCCCACAA | 95 | ChrX_ERV_denovo:51 |
| BosT ERV PC3-R | ACCGTCGT | | 33-5154 |
| Oar JSRV Pool A | | | |
| 2625-JSRV-5PA-F | TTTCTGTTGGTGCTGATATTGGCCTCCACCGTCTGA | 96 | enJSRV-7:1269-1290 |
| | GAACATGT | | |
| 2626-JSRV-5PA-R | ACTTGCCTCTCCCTCTATCTTCAGCATACCTGGGTT | 97 | enJSRV-7:920-941 |
| | CCGAATCA | | |
| 2640-JSRV-3PA-F | TTTCTGTTGGTGCTGATATTGCGAACCGGACCTCTC | 98 | enJSRV-7:6216-6237 |
| | GACATTCC | | |
| 2641-JSRV-3PA-R | AGTTGCCTGTCGCTCTATCTTCAAACACAAACATGC | 99 | enJSRV-7:5650-5671 |
| | CCTCGTCC | | |
| Oar JSRV Pool B | | | |
| 2630-JSRV-5PB-F | TTTCTGTTGGTGCTGATATTGCGGGACCTGATGAGC | 100 | enJSRV-7:1796-1817 |
| | CTTACCAG | | |
| 2631-JSRV-5PB-R | ACTTGCCTGTCGCTCTATCTTCGCAATGGTGAATGG | 101 | enJSRV-7:1453-1474 |
| | AGCGGTAG | | |
| 2635-JSRV-3PB-F | TTTCTGTTGGTGCTGATATTGCCCTTCATTCACTGT | 102 | enJSRV-7:7306-7327 |
| | GGCGAAGT | | |
| 2636-JSRV-3PB-R | ACTTGCCTGTCGCTCTATCTTCGTAAGGAACACAAG | 103 | enJSRV-7:6553-6574 |
| | CTCGGGGA | | |
| HTLV1 Pool A | | | |
| 2607-HTLV1-5PA-F | TTTCTGTTGGTGCTGATATTGCTCATCCAAACCCAA | 104 | HTLV_ATK:1083-1104 |
| | GCCCAGAT | | |
| 2608-HTLV1-5PA-R | ACTTGCCTGTCGCTCTATCTTCGGACCGGGTTCTAG | 105 | HTLV_ATK:915-936 |
| | GCGATATG | | |
| 2609-HTLV1-3PA-F | TTTCTGTTGGTGCTGATATTGCTCTACCCGAAGACT | 106 | HTLV_ATK:7941-7962 |
| | GTTTGCCC | | |
| 2610-HTLV1-3PA-R | ACTTGCCTCTCCCTCTATCTTCTTGTATGAGTGATT | 107 | HTLV_ATK:7591-7612 |
| | GGCGGGGT | | |
| HTLV1 Pool B | | | |
| 2614-HTLV1-5PB-F | TTTGTGTTGGTGCTGATATTGCAAAGACCTCCAAGA | 108 | HTLV_ATK:1370-1391 |
| | CCTCCTGC | | |
| 2615-HTLV1-5PB-R | ACTTGCCTGTCGCTCTATCTTCCGTAGGCTCAACAT | 109 | HTLV_ATK:1177-1198 |
| | AGGGAGGG | | |
| 2623-HTLV1-3PB-F | TTTCTGTTGGTGCTGATATTGGCTCTCACACGGCCT | 110 | HTLV_ATK:8194-8215 |
| | CATACAGT | | |
| 2624-HTLV1-3PB-R | ACTTGCCTGTCGCTCTATCTTCGAGTGGTGAGGGTT | 111 | HTLV_ATK:8029-8050 |
| | GAGTGGAA | | |
| HIV UI Pool A | | | |
| 2665-HIV-5PA-F | TTTCTGTTGGTGCTGATATTGCAaaattcggttaag | 112 | HIV_U1:841-862 |
| | gccagggg | | |
| 2666-HIV-5PA-R | ACTTGCCTGTCGCTCTATCTTCCtcgcacccatctc | 113 | HIV_U1:779-800 |
| | tctccttc | | |
| 2679-HIV-3PA-F | TTTCTGTTGGTGCTGATATTGCGctaccaccgcttg | 114 | HIV_U1:8461-8482 |
| | agagactt | | |
| 2680-HIV-3PA-R | ACTTGCCTGTCGCTCTATCTTCaccaattccacaaa | 115 | HIV_U1:8157-8178 |
| | cttgccca | | |
| HIV UI Pool B | | | |
| 2669-HIV-5PB-F | TTTCTGTTGGTGCTGATATTGCCcaggccagatgag | 116 | HIV_U1:1462-1483 |
| | agaaccaa | | |
| 2670-HIV-5PB-R | ACTTGCCTGTCGCTCTATCTTCtcccattctgcagc | 117 | HIV_U1:1406-1427 |
| | ttcctcat | | |
| 2674-HIV-3PB-F | TTTCTGTTGGTGCTGATATTGCgaggaggaggaggt | 118 | HIV_U1:8917-8938 |
| | gggttttc | | |
| 2675-HIV-3PB-R | ACTTGCCTGTCGCTCTATCTTCtgaccacttgccac | 119 | HIV_U1:8730-8751 |
| | ccatctta | | |
| HPV18 Pool A | | | |
| 4_HPV18_R1_Left | ctccaacgacgcagagaaacac | 120 | |
| 5_HPV18_R1_Right | ggattcaacggtttctggcacc | 121 | |
| HPV18_Pool_B | | | |
| 11_HPV18_R2_Left | ttttggttcaggctggattgcg | 122 | |
| 12_HPV18_R2_Right | agaatacacacagctgccaggt | 123 | |
| HPV16_E6-E7 | | | |
| 3259_HPV16_E6-E7 | AACCGGACAGAGCCCATTACAA | 124 | |
| 3260_HPV16_E6-E7 | AGTCATATACCTCACGTCGCAGT | 125 | |
| HPV16_L1 | | | |
| 3264_HPV16_L1 | ACTGGCTTTGGTGCTATGGACT | 126 | |
| 3265_HPV16_L1 | CAAACCAGCCGCTGTGTATCTG | 127 | |

Identification of Proviral Integration Sites in PCIP-Seq

Reads were mapped with Minimap2⁵⁵ to the host genome with the proviral genome as a separate chromosome. In-house R-scripts were used to identify integration sites (IS). Briefly, chimeric reads that partially mapped to at least one extremity of the proviral genome were used to extract virus-host junctions and shear sites. Junctions within a 200 bp window were clustered together to form an “IS cluster”, compensating for sequencing/mapping errors. The IS retained corresponded to the position supported by the highest number of virus-host junctions in each IS cluster. Clone abundance was estimated based on the number of reads supporting each IS cluster. Reads sharing the same integration site and same shear site were considered PCR duplicates. Custom software, code description and detailed outline of the workflow are available on Github: https://github.com/GIGA-AnimalGenomics-BLV/PCIP.

Measure of Proviral Load (PVL) and Identification of Proviral Integration Sites (Illumine)

PVLs and integration sites of HTLV-1- and BLV-positive individuals were determined as previously described in Rosewick et al 2017⁷ and Artesi et al 2017²⁰. PVL represents the percentage of infected cells, considering a single proviral integration per cell. Total HIV-1 DNA content of CD4 T-cell DNA isolates was measured by digital droplet PCR (ddPCR, QX200 platform, Bio-Rad, Temse, Belgium), as described by Rutsaert et al.⁵⁶ The DNA was subjected to a restriction digest with EcoRI (Promega, Leiden, The Netherlands) for one hour, and diluted 1:2 in nuclease free water. HIV-1 DNA was measured in triplicate using 4 μL of the diluted DNA as input into a 20 μL reaction, while the RPP30 reference gene was measured in duplicate using 1 μL as input. Primers and probes are summarized in Table 3. Thermocycling conditions were as follows: 95° C. for 10 min, followed by 40 cycles of 95° C. for 30 s and 56° C. for 60 s, followed by 98° C. for 10 min. Data was analyzed with the ddpcRquant analysis software⁵⁷.

TABLE 3

| | Loca- | | | Temp. | |
|---|---|---|---|---|---|
| Assay | tion | Primer | Label | (° C.) | Sequence |
| Total | HIV | Forward | MGB/ | 56 | 5′-GCCTCAATA |
| HIV-1 | LTR | | FAM | | AAGCTTGCC-3′ |
| DNA | | | | | (SEQ ID |
| | | | | | NO: 128) |
| | HIV | Reverse | | | 5′-GGCGCCAC |
| | LTR- | | | | TGCTAGAGATT |
| | Gag | | | | TT-3^{′} |
| | inter | | | | (SEQ ID |
| | | | | | NO: 129) |
| | HIV | Probe | | | 5′-AAGTRGTG |
| | LTR | | | | TGTGCCC-3^{′} |
| | | | | | (SEQ ID |
| | | | | | NO: 130) |
| RPP30 | human | Forward | HEX | 56 | 5′-AGATTTGGA |
| | RPP30 | | | | CCTGCGAGCG-3′ |
| | gene | | | | (SEQ ID |
| | | | | | NO: 131) |
| | human | Reverse | | | 5′-GAGCGGCTGT |
| | RPP30 | | | | CTCCACAAGT-3′ |
| | gene | | | | (SEQ ID |
| | | | | | NO: 132) |
| | human | Probe | | | 5′-TTCTGACCTG |
| | RPP30 | | | | AAGGCTCTGCGC |
| | gene | | | | G-3′ |
| | | | | | (SEQ ID |
| | | | | | NO: 133) |

Variant Calling

After PCR duplicate removal, proviruses with an IS supported by more than 10 reads were retained for further processing. SNPs were identified using LoFreq²² with default parameters, only SNPs with an allele frequency of >0.6 in the provirus associated with the insertion site were considered. We also called variants on proviruses supported by more than 10 reads without PCR duplicate removal (this greatly increased the number of proviruses examined). This data was used to explore the number of proviruses carrying the Tax 303 variant. Deletions were called on proviruses supported by more than 10 reads without PCR duplicate removal using an in house R-scripts. Briefly, samtools pileup⁵⁸ was used to calculate/compute coverage and deletions at base resolution. We used the changepoint detection algorithm PELT⁵⁹ to identify genomic windows showing an abrupt change in coverage. Windows that showed at least a 4-fold increase in the frequency of deletions (absence of a nucleotide for that position within a read) were flagged as deletions and visually confirmed in IGV⁸⁰.

HIV-1 Proviral Sequences

Sequences of the two major proviruses integrated in chr2 (SEQ ID NO:5) and chrX (SEQ ID NO:4) of the U1 cell line were generated by initially mapping the reads from both platforms to the HIV-1 provirus, isolate NY5 (GenBank: M38431.1), where the 5′LTR sequence is appended to the end of the sequence to produce a full-length HIV-1 proviral genome reference. The sequence was then manually curated to produce the sequence for each provirus. To check for recombination, reads of selected clones were mapped to the sequence from the chrX provirus and the patterns of SNPs examined to determine if the variants matched the chrX or chr2 proviruses.

Endogenous Retroviruses

The sequence of bovine APOB ERV (SEQ ID NO:6) was generated by PCR amplifying the full length ERV with LongAmp Taq DNA Polymerase (New England Biolabs) from a Holstein suffering from cholesterol deficiency. The resultant PCR product was sequenced on the Illumina platform as described below. It was also sequenced with an Oxford Nanopore MinION R7 flow cell as previously described²⁹. Full length sequence of the element was generated via manual curation. Guide RNAs and primer pairs were designed using this ERV reference. For the Ovine ERV we used the previously published enJSRV-7 sequence⁴⁰ as a reference to design PCIP-seq guide RNAs and PCR primers.

As the ovine and bovine genome contains sequences matching the ERV, mapping ERV PCIP-seq reads back to the reference genome creates a large pileup of reads in these regions. To avoid this, prior to mapping to the reference we first used BLAST⁶¹ to identify the regions in the reference genome containing sequences matching the ERV, we then used BEDtools⁶² to mask those regions. The appropriate ERV reference was then added as an additional chromosome in the reference.

PCR validation and Illumina Sequencing

Clone specific PCR products were generated by placing primers in the flanking DNA as well as inside the provirus. LongAmp Taq DNA Polymerase (New England Biolabs) was used for amplification following the manufacturers guidelines. Resultant PCR products were sheared to ˜400 bp using the Bioruptor Pico (Diagenode) and Nextera XT indexes added as previously described²⁹. Illumina PCIP-seq libraries were generated in the same manner. Sequencing was carried out on either an Illumina MiSeq or NextSeq 500. Clone specific PCR products sequenced on Nanopore were indexed by PCR, multiplexed and libraries prepared using the Ligation Sequencing Kit 1D (SQK-LSK108) and sequenced on a MinION R9.4 flow cell. Oligos used can be found in Tables 4-7.

TABLE 4
Primers used for clone specific validation of SNPs
Ovine 220_122013

| | | | | | Oligo | | |
|---|---|---|---|---|---|---|---|
| | POS in | | | | location | | |
| | BLV | | | | in BLV | | Location in |
| Provirus | genome | REF | ALT | BLV Oligo | Provirus | Host Oligo | Host |
| OAR12_62009791_ | 7925 | T | G | TTTCAGAGGGCGGAGA | 4648-4667 | CACCCTGAGCCTCCATA | chr12:62010099- |
| 62009791 | | | | AACA | | CAT | 62010118 |
| | | | | (SEQ ID NO: 134) | | (SEQ ID NO: 137) | |
| OAR2_248506820_ | 466 | T | C | TTTAGCAAACGCCAGG | 4797-4816 | GCGAATCTCTGTCTTGC | chr2:248506994- |
| 248507220 | | | | GAAC | | TGG | 248507013 |
| | | | | (SEQ ID NO: 135) | | (SEQ ID NO: 138) | |
| OAR5_60508711_ | 7511 | G | A | TTTCAGAGGGCGGAGA | 4648-4667 | AACTCTATGGCTGGAAG | chr5:60509280- |
| 60508719 | | | | AACA | | GACA | 60509300 |
| | | | | (SEQ ID NO: 136) | | (SEQ ID NO: 139) | |

Ovine 221_022016 & 221_032014

| | | | | | Oligo | | |
|---|---|---|---|---|---|---|---|
| | POS in | | | | location | | |
| | BLV | | | | in BLV | | |
| Provirus | genome | REF | ALT | BLV Oligo | Provirus | Host Oligo | Location |
| OARX_115780553_ | 6251 | G | A | TTTCAGAGGGCGGAGA | 4648-4667 | AGGTGGAGATGATGTG | chrX:115781164- |
| 115780560 | | | | AACA | | TGC A | 115781183 |
| | | | | (SEQ ID NO: 140) | | (SEQ ID NO: 146) | |
| OAR3_68849355_ | 973 | G | A | TTTAGCAAACGCCAGG | 4797-4816 | ACCTCACACCAAAACGA | chr3:68849738- |
| 68850177 | | | | GAAC | | AGC | 68849757 |
| | | | | (SEQ ID NO: 141) | | (SEQ ID NO: 147) | |
| ″ | 2917 | G | A | ″ | ″ | ″ | ″ |
| ″ | 3139 | C | T | ″ | ″ | ″ | ″ |
| OAR8_80138768_ | 3407 | T | C | TTTAGCAAACGCCAGG | 4797-4816 | GTGACTTGTTTGCCTCCC | chr8:80137900- |
| 80138775 | | | | GAAC | | TG | 80137919 |
| | | | | (SEQ ID NO: 142) | | (SEQ ID NO: 148) | |
| OAR2_56698159_ | 7524 | C | A | TTTAGCAAACGCCAGG | 4797-4816 | TTCATGTGCTTCCGTGG | chr2:56698504- |
| 56698164 | | | | GAAC | | TTG | 56698523 |
| | | | | (SEQ ID NO: 143) | | (SEQ ID NO: 149) | |
| OAR7_72660067_ | 7191 | G | A | TTTCAGAGGGCGGAGA | 4648-4667 | AGAGGCCTGAGTGTTTT | chr7:72660692- |
| 37266007 | | | | AACA | | GGT | 72660711 |
| | | | | (SEQ ID NO: 144) | | (SEQ ID NO: 150) | |
| OAR8_80151001_ | 5305 | G | A | TTTCAGAGGGCGGAGA | 4648-4667 | GACCCACATCAGTTGCC | chr8:80151348- |
| 78015100 | | | | AACA | | TTC | 80151367 |
| | | | | (SEQ ID NO: 145) | | (SEQ ID NO: 151) | |

Bovine 14³9

| | | | | | Oligo | | |
|---|---|---|---|---|---|---|---|
| | POS in | | | | location | | |
| | BLV | | | | in BLV | | |
| Provirus | genome | REF | ALT | BLV Oligo | Provirus | Host Oligo | Location |
| 24_41573470_ | 3415 | A | G | GGGGCTCGCAATCATA | 5143-5162 | CTTGAACTCCGGGACCT | chr24:41574183- |
| 41573476 | | | | TGTG | | TCT | 41574202 |
| | | | | (SEQ ID NO: 152) | | (SEQ ID NO: 166) | |
| 22_48070162_ | 3470 | T | G | GGGGCTCGCAATCATA | 5143-5162 | TCGAAAAGGCCAAGTAC | chr22:48070630- |
| 48070168 | | | | TGTG | | CCT | 48070649 |
| | | | | (SEQ ID NO: 153) | | (SEQ ID NO: 167) | |
| 18_57045658_ | 3440 | T | C | GGGGCTCGCAATCATA | 5143-5162 | GATGGGATGAGGTCAG | chr18:57045372- |
| 57045664 | | | | TGTG | | GAGG | 57045391 |
| | | | | (SEQ ID NO: 154) | | (SEQ ID NO: 168) | |
| 18_61039250_ | 453 | T | C | GGGGCTCGCAATCATA | 5143-5162 | ACAGGCAGGATCTTTGT | chr18:61039161- |
| 61039250 | | | | TGTG | | GGA | 61039180 |
| | | | | (SEQ ID NO: 155) | | (SEQ ID NO: 169) | |
| 2_5529599_ | 106 | C | T | GGGGCTCGCAATCATA | 5143-5162 | GCACACTGTCCTGAGAtc | chr2:5529276- |
| 5529704 | | | | TGTG | | ca | 5529295 |
| | | | | (SEQ ID NO: 156) | | (SEQ ID NO: 170) | |
| ″ | 8295 | C | T | AGCCCTCTGGACTCACA | 4562-4581 | CCAGTGCATGCttaat | chr2:5530006- |
| | | | | ATC | | cgct | 5530025 |
| | | | | (SEQ ID NO: 157) | | (SEQ ID NO: 171) | |
| 2_54238495_ | 93 | T | C | GGGGCTCGCAATCATA | 5143-5162 | AATCCGTTCATGGTTCC | chr2:54238966- |
| 54238502 | | | | TGTG | | GTG | 54238985 |
| | | | | (SEQ ID NO: 158) | | (SEQ ID NO: 172) | |
| ″ | 7437 | T | C | AGCCCTCTGGACTCACA | 4562-4581 | GCTGCTAATTTGACTGG | chr2:54237331- |
| | | | | ATC | | CCA | 54237350 |
| | | | | (SEQ ID NO: 159) | | (SEQ ID NO: 173) | |
| ″ | 8282 | T | C | ″ | ″ | ″ | ″ |
| 21_45410573_ | 2885 | c | A | GGGGCTCGCAATCATA | 5143-5162 | CTCGGGGAGACAGAAA | chr21:45410493- |
| 45410985 | | | | TGTG | | ACCT | 45410512 |
| | | | | (SEQ ID NO: 160) | | (SEQ ID NO: 174) | |
| 29_41063804_ | 3662 | A | G | AGCCCTCTGGACTCACA | 4562-4581 | CTTCCCTGCTCCATCCCT | chr29:41062629- |
| 41063804 | | | | ATC | | AG | 41062648 |
| | | | | (SEQ ID NO: 161) | | (SEQ ID NO: 175) | |
| ″ | 8642 | T | C | GGGGCTCGCAATCATA | 5143-5162 | CAGCTTACTCCACCCTTC | chr29:41064575- |
| | | | | TGTG | | CA | 41064594 |
| | | | | (SEQ ID NO: 162) | | (SEQ ID NO: 176) | |
| 3_87619443_ | 453 | T | C | AGCCCTCTGGACTCACA | 4562-4581 | GCAAGAGAAGAGAGTG | chr3:87618300- |
| 87619450 | | | | ATC | | GGGT | 87618319 |
| | | | | (SEQ ID NO: 163) | | (SEQ ID NO: 177) | |
| ″ | 8642 | T | C | GGGGCTCGCAATCATA | 5143-5162 | TCTAATCCCCAAGCTGT | chr3:87619588- |
| | | | | TGTG | | GCA | 87619607 |
| | | | | (SEQ ID NO: 164) | | (SEQ ID NO: 178) | |
| 1_150385145_ | 5859 | G | A | AGCCCTCTGGACTCACA | 4562-4581 | CGACAAGCCTGGTAAG | chr1:150385624- |
| 150385351 | | | | ATC | | ATGC | 150385643 |
| | | | | (SEQ ID NO: 165) | | (SEQ ID NO: 179) | |

TABLE 5
Primers for clone specific validation of SV
Bovine 1439

| | Aprox | Aprox | | | Oligo locaion | | Location |
|---|---|---|---|---|---|---|---|
| | start | end | | | in BLV | | in |
| Provirus | BLV | BLV | type | BLV Oligo | Provirus | Host Oligo | Host |
| 1_150385145_ | 3451 | 3474 | DE | GGGGCTCGCAATCATA | 5143-5162 | GTGGGACGGTGTTTGA | chr1:150384631- |
| 150385351 | | | L | TGTG | | AGTC | 150384650 |
| | | | | (SEQ ID NO: 180) | | (SEQ ID NO: 188) | |
| 2_124084208_ | 391 | 406 | DE | GAGGCATCGATAGCAT | 1663-1684 | TTCCCCAAGACTTTCCC | chr2:124084230- |
| 124084213 | | | L | GGTCC.T | | AGGTC | 124084251 |
| | | | | (SEQ ID NO: 181) | | (SEQ ID NO: 189) | |
| 23_39892380_ | 2364 | 2560 | DE | AAATCTGGGGCCACAA | 3504-3525 | TCCAGTGGCCGTGTAT | chr23:39893192- |
| 39892560 | | | L | TTGCAG | | TTGTCT | 39893213 |
| | | | | (SEQ ID NO: 182) | | (SEQ ID NO: 190) | |
| 27_36582809_ | 1 | 852 | DE | CCACCCTATTGCTTCC | 3950-3969 | TTCCCTTAGCAGTCAG | chr27:36583265- |
| 36582809 | | | L | CTGA | | GTGG | 36583284 |
| | | | | (SEQ ID NO: 183) | | (SEQ ID NO: 191) | |
| 27_36582809_ | 4522 | 5636 | DE | GGCATGAGTAGCTCCA | 4258-4277 | AGGCCTTCACTCTAACC | chr27:36581475- |
| 36582809 | | | L | GAGT | | GTT | 36581494 |
| | | | | (SEQ ID NO: 184) | | (SEQ ID NO: 192) | |
| 3_45576532_ | 2316 | 2336 | DE | AAATCTGGGGCCACAA | 3504-3525 | TACTGCCCATCACCCCT | chr3:45576400- |
| 45576538 | | | L | TTGCAG | | TCATC | 45576421 |
| | | | | (SEQ ID NO: 185) | | (SEQ ID NO193) | |
| 4_100234239_ | 8296 | 8370 | INS | AGCCCTCTGGACTCAC | 4562-4581 | ACAAAACAGTCAAACA | chr4:100234688- |
| 100234246 | | | | AATC | | GGGCT | 100234708 |
| | | | | (SEQ ID NO: 186) | | (SEQ ID NO: 194) | |
| 5_51456241_ | 1 | 4152 | DE | AGCGAGGAGAGTGAG | 4882-4903 | CCCCTGCATAAAATGA | chr5:51456399- |
| 51456285 | | | L | AGTGAGA | | GGCCTG | 51456420 |
| | | | | (SEQ ID NO: 187) | | (SEQ ID NO: 195) | |

Ovine 221

| | Aprox | Aprox | | | Oligo locaion | | Location |
|---|---|---|---|---|---|---|---|
| | start | end | | | in BLV | | in |
| Provirus | BLV | BLV | type | BLV Oligo | Provirus | Host Oligo | Host |
| OAR25_25097056_ | 2325 | 4303 | DE | AGATTTCAGGGAAGTG | 6236-6257 | TGCCTTCTCCGTTCCCA | chr25:25097010- |
| 25097063 | | | L | GGGAGC | | ATTCT | 25097031 |
| | | | | (SEQ ID NO: 196) | | (SEQ ID NO: 202) | |
| OARX_78143793_ | 3284 | 6602 | DE | TGGATGTGGCTGGAAT | 7063-7082 | CACCAGGGAAGTCTTG | chrX:78144637- |
| 78143801 | | | L | GTCT | | TTGC | 78144656 |
| | | | | (SEQ ID NO: 197) | | (SEQ ID NO: 203) | |
| OARX_78143793_ | 3284 | 6602 | DE | AATTACAGGCGGTCTT | 3025-3044 | CAGCCTCAGAGTTCCTT | chrX:78143342- |
| 78143801 | | | L | GGGA | | CCA | 78143361 |
| | | | | (SEQ ID NO: 198) | | (SEQ ID NO: 204) | |
| OAR1_250672128_ | 7365 | 7389 | DE | AAATGCCCAAAGAACG | 4824-4845 | AGCCTTCACAAGTCAC | chr1:250672354- |
| 250672136 | | | L | ACGGTC | | CTCTCC | 250672375 |
| | | | | (SEQ ID NO: 199) | | (SEQ ID NO: 205) | |
| OAR2_242159705_ | 7017 | 7232 | INS | CGAATCTTCCCCATGCA | 6775-6796 | GATGCCCTGGAATGGT | chr2:242159088- |
| 242159712 | | | | GCTTC | | TTGGTG | 242159109 |
| | | | | (SEQ ID NO: 200) | | (SEQ ID NO: 206) | |
| OAR8_80161637_ | 6502 | 6561 | DE | AAATGCCCAAAGAACG | 4824-4845 | TCCAGAAGAGGCAAAG | chr8:80163636- |
| 80161982 | | | L | ACGGTC | | CAAGGA | 80163657 |
| | | | | (SEQ ID NO: 201) | | (SEQ ID NO: 207) | |

Ovine 223

| | | | | | Position of | | |
|---|---|---|---|---|---|---|---|
| | Aprox | Aprox | | | oligo | | Location |
| | start | end | | | in BLV | | in |
| Provirus | BLV | BLV | type | BLV Oligo | Provirus | Host Oligo | Host |
| OAR10_34545991_ | 5298 | 5330 | DE | AAATGCCCAAAGAACG | 4824-4845 | AAGTCGAGCAAGGCAC | chr10:34547689- |
| 34546003 | | | L | ACGGTC | | CTATGT | 34547710 |
| | | | | (SEQ ID NO: 208) | | (SEQ ID NO: 210) | |
| OAR10_49266255_ | 6512 | 6586 | DE | AAATGCCCAAAGAACG | 4824-4845 | TGGTTGTGGGTCATCA | chr1O:492663OO- |
| 49266262 | | | L | ACGGTC | | TCGTCT | 49266321 |
| | | | | (SEQ ID NO: 209) | | (SEQ ID NO: 211) | |

TABLE 6
Primers for long range PCR to validate
ERVs in the Bovine

| | Forward | | Reverse | |
|---|---|---|---|---|
| ERV | Oligo | Location | Oligo | Location |
| BTA8_ | GGCTGCC | chr8: | TTTACCC | chr8: |
| 37.3 | CTTCACT | 37362441- | TTGGAGT | 37362889- |
| | GAGAGTAA | 37362462 | GTGGCCTT | 37362910 |
| | (SEQ ID | | (SEQ ID | |
| | NO: 212) | | NO: 215) | |
| BTA21 | TGGCTAAG | chr21: | GGGTCCT | chr21: |
| _18.6 | TTCCAC | 18639407- | CTGTCCT | 18639907- |
| | CACACTCT | 18639428 | CTGTCTTC | 18639928 |
| | (SEQ ID | | (SEQ ID | |
| | NO: 213) | | NO: 216) | |
| BTA27 | GGAGCAA | chr27: | AGAGGGA | chr27: |
| _14.1 | GGTAGAG | 14152640- | AATCAC | 14153202- |
| | GGGTGAAG | 14152661 | ACCGAAG | 14153223 |
| | (SEQ ID | | CA | |
| | NO: 214) | | (SEQ ID | |
| | | | NO: 217) | |

TABLE 7
Primers for long range PCR to
validate ERVs in the Ovine

| | Forward | | Reverse | |
|---|---|---|---|---|
| ERV | Oligo | Location | Oligo | Location |
| OAR1_ | GTTGTTG | chr1: | GGAGCCT | chr1: |
| 86.0 | CATCTTC | 85959032- | CAACGAC | 85964651- |
| | CGGTCCTG | 85959053 | TCTGCTAA | 85964672 |
| | (SEQ ID | | (SEQ ID | |
| | NO: 218) | | NO: 225) | |
| OAR3_ | TAGCCCA | chr3: | CCCCTTC | chr3: |
| 39.2 | GCAAGAG | 39184853- | ATAGCCC | 39196544- |
| | TCTCCCTA | 39184874 | ACTGGAAA | 39196565 |
| | (SEQ ID | | (SEQ ID | |
| | NO: 219) | | NO: 226) | |
| OAR4_ | TTGATGT | chr4: | CCAGCAA | chr4: |
| 77.4 | GAAGAGC | 77421367- | CTCAGAC | 77421696- |
| | CTGTGAGC | 77421388 | AAACCAGG | 77421717 |
| | (SEQ ID | | (SEQ ID | |
| | NO: 220) | | NO: 227) | |
| OAR13 | GGCTTCA | chr13: | AATGTGTA | chr13: |
| _16.7 | AACACAC | 16720272 | GATGGAG | 16721090 |
| | CTCACCT | -16720293 | GCTGGGC | -16721111 |
| | C | | (SEQ ID | |
| | (SEQ ID | | NO: 228) | |
| | NO: 221) | | | |
| OAR4_ | GAGATGG | chr4: | GCTAACA | chr4: |
| 40.4 | CCGTGT | 40492573- | AACGGGT | 40493498- |
| | GTGACA | 40492594 | GGCAAAGA | 40493519 |
| | AAG | | (SEQ ID | |
| | (SEQ ID | | NO: 229) | |
| | NO: 222) | | | |
| OAR5_ | TGAAAGA | chr5: | CTGGGGA | chr5: |
| 73.0 | CTCACTG | 73012745- | AGCCAA | 73013599- |
| | TGGCCCAA | 73012766 | GCAAAGATG | 73013620 |
| | (SEQ ID | | (SEQ ID | |
| | NO: 223) | | NO: 230) | |
| OAR13 | ACTCTCTC | chr13: | ATTCTGGT | chr13: |
| _66.0 | CCAACAT | 66026352 | GGTCTC | 66027161 |
| | TGCCCTC | -66026373 | TGTGGCTC | -66027182 |
| | (SEQ ID | | (SEQ ID | |
| | NO: 224) | | NO: 231) | |

BLV References

The sequence (SEQ ID NO:1) of the pBLV344 provirus was generated via a combination of Sanger and Illumina based sequencing with manual curation of the sequence to produce a full length proviral sequence. The consensus BLV sequences for the bovine samples 1439 & 1053 (SEQ ID NO:3,2) were generated by first mapping the PCIP-seq Nanopore reads to the pBLV344 provirus. We then used Nanopolish⁶³ to create an improved consensus. PCIP-seq libraries sequenced on the Illumina and Nanopore platform were mapped to this improved consensus visualized in IGV and manually corrected.

Genome References Used

Sheep=OAR3.1; Cattle=UMD3.1; Human=hg38; For HTLV-1 integration sites hg19 was used; HPV18=GenBank: AY262282.1; Sequences of the exogenous and endogenous proviruses can be found in SEQ ID NO:1-SEQ ID NO:6.

Data Availability

Sequence data that support the findings of this study have been deposited in the European Nucleotide Archive (ENA) hosted by the European Bioinformatics Institute (EMBL-EBI) and are accessible through study accession number PRJEB34495. All other relevant data are available within the article and its Supplementary Information files or from the corresponding authors upon reasonable request.

Code Availability

The code and a detailed outline of the PCIP-seq analysis workflow are publicly available on Github: https://github.com/GIGA-AnimalGenomics-BLV/PCIP

### Example 2: Overview of PCIP-Seq (Pooled CRISPR Inverse PCR-Sequencing)

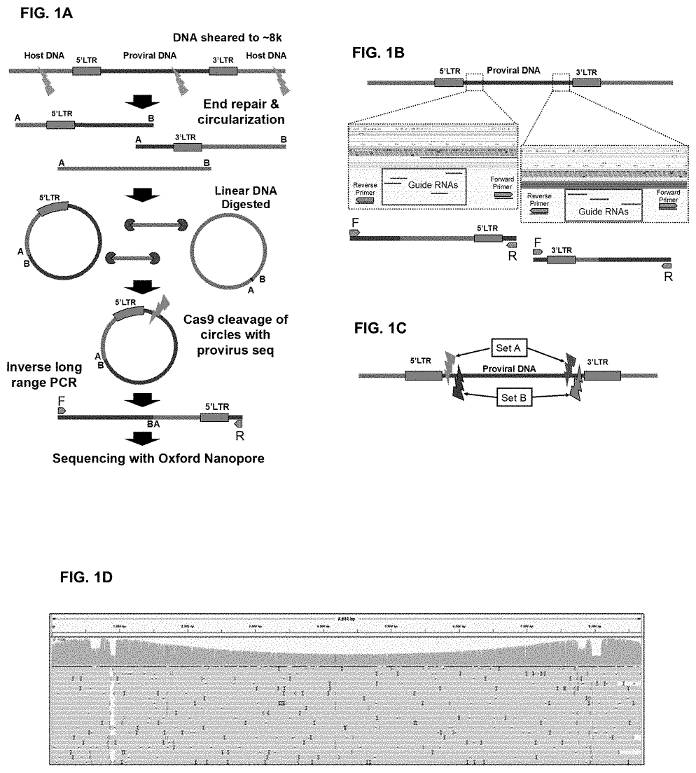

The genome size of the viruses targeted ranged from 6.8 to 9.7 kb, therefore we chose to shear the DNA to ˜8 kb in length. In most cases this creates two fragments for each provirus, one containing the 5′ end with host DNA upstream of the insertion site and the second with the 3′ end and downstream host DNA. Depending on the shear site the amount of host and proviral DNA in each fragment will vary (FIG. 1a). To facilitate identification of the provirus insertion site via inverse PCR we carry out intramolecular ligation, followed by digestion of the remaining linear DNA. To selectively linearize the circular DNA containing proviral sequences (this helps increase PCR efficiency), regions adjacent to the 5′ and 3′ LTRs in the provirus are targeted for CRISPR mediated cleavage. We sought a balance between ensuring that the majority of the reads contained part of the flanking DNA (for clone identification) while also generating sufficient reads extending into the midpoint of the provirus. We found that using a pool of CRISPR guides for each region increased the efficiency and by multiplexing the guide pools and PCR primers for the 5′ and 3′ ends we could generate coverage for the majority of a clonally expanded provirus in a single reaction (FIG. 1b). The multiplexed pool of guides and primers leaves coverage gaps in the regions flanked by the primers. To address these coverage gaps we designed a second set of guides and primers. Following separate CRISPR cleavage and PCR amplification the products of these two sets of guides and primers were combined for sequencing (FIG. 1c). This approach ensured that the complete provirus was sequenced (FIG. 1d).

Pooled CRISPR Inverse PCR sequencing (PCIP-seq) leverages long reads on the Oxford Nanopore MinION platform to sequence the insertion site and its associated provirus. The technique was applied to natural infections produced by three exogenous retroviruses, HTLV-1, BLV and HIV-1 as well as endogenous retroviruses in both cattle and sheep. The high efficiency of the method facilitated the identification of tens of thousands of insertion sites in a single sample. Thousands of SNPs and dozens of structural variants within proviruses were observed. While initially developed for retroviruses the method has also been successfully extended to DNA extracted from HPV positive PAP smears, where it could assist in identifying viral integrations associated with clonal expansion. An overview of the applications tested herein is provided in Table 8.

TABLE 8
Number of insertion sites (IS) identified via PCIP-seq. Chimeric reads = reads containing host and viral
DNA. Largest clone % = insertion site with highest number of reads in that sample. PVL = Proviral
Load. (Percentage cells carrying a single copy of integrated provirus or number proviral copies per 100 cells).

| | | | | | | | Pure | | |
|---|---|---|---|---|---|---|---|---|---|
| | | | | | | Chimeric | Host/Pure | | Largest |
| Sample | | | | Template | raw | reads | Viral | Insertion | clone |
| name | Virus | Host | PVL | μg | reads | (%) | reads | sites | (%) |
| ATL2 | HTLV-1 | HSA | nd | 4 | 81,219 | 68.21 | 0.0037/31.8 | 160 | 49.5 |
| ATL100 | HTLV-1 | HSA | 106 | 4 | 4,838 | 64.14 | 9.16/26.7 | 13 | 89.624 |
| 233 | BLV | OAR | 78.3 | 7 | 524,698 | 53.4 | 0.04/46.53 | 5311 | 5.22 |
| 221 (022016) | BLV | OAR | 63 | 4 | 180,276 | 67.14 | 3.59/29.27 | 8023 | 0.625 |
| 221 (032014) | BLV | OAR | 16 | 4 | 32,266 | 68.69 | 0.11/31.20 | 5374 | 0.279 |
| 220 | BLV | OAR | 3.8 | 2 | 44,876 | 67.38 | 0/32.62 | 1352 | 3.55 |
| 1439 | BLV | BosT | 45 | 3 | 181,055 | 70.52 | 0.19/29.29 | 5773 | 1.17 |
| 560 | BLV | BosT | 0.644 | 1 | 6,802 | 69.83 | 1.12/29.06 | 172 | 4.59 |
| 1053 | BLV | BosT | 23.5 | 6 | 367,454 | 72.13 | 0.04/27.83 | 17903 | 0.353 |
| HIV_U1 | HIV-1 | HSA | 200 | 2 | 94,086 | 54.66 | 2.75/42.59 | 728 | 47.2 |
| Jurkat U1-0.1 | HIV-1 | HSA | 0.2 | 5 | 252,913 | 43.33 | 0.04/56.62 | 4 | 71.7 |
| Jurkat U1-0.01 | HIV-1 | HSA | 0.02 | 5 | 234,421 | 43.33 | 0.04/56.52 | 2 | 90.2 |
| Jurkat neg | HIV-1 | HSA | 0 | 5 | 12,137 | 0 | 100/0 | 0 | 0 |
| HPV18_PX | HPV18 | HAS | nd | 4 | 180,550 | 21.36 | 0.29/78.35 | 55 | nd |
| HPV18_PY | HPV18 | HAS | nd | 4 | 82,807 | 0.09 | 0.05/99.86 | 19 | nd |

### Example 3: Rationale Behind the Use of CRISPR-Cas9 to Cleave Circular DNA

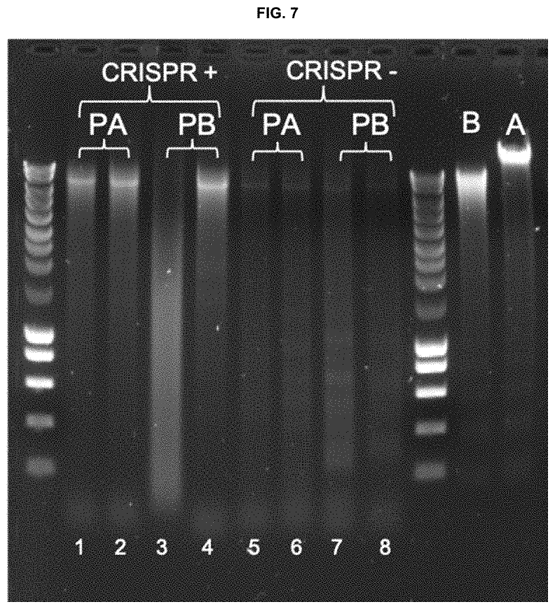

It is established practice to linearize plasmids (generally via cutting with a restriction enzyme) prior to their use as template in PCR. It is believed that this avoids supercoiling and thereby increases PCR efficiency⁶⁷. Following the same logic, we speculated that linearizing our circularized DNA could also increase PCR efficiency. FIG. 7 shows an experiment carried out using 8 μg of DNA from a BLV infected sheep with a proviral load of 82.6%. The DNA was circularized and linear DNA was eliminated (to prevent PCR amplification/recombination involving the remaining linear fragments) using plasmid safe DNase (see Example 1 for a complete description). One quarter of the resultant DNA was subject to CRISPR-cas9 cleavage using the Pool A guides, the second quarter was cleaved using the Pool B guides, the remaining half was kept aside. The linearized DNA was cleaned and used as template in 2×50 μl PCR reactions using the appropriate primer pairs for Pool A (PA) or Pool B (PB). For the uncut DNA half was used as template for 2×50 μl PCR reactions using the PA primers and the other half was used for 2×50 μl PCR reactions using the PB primers. Following 25 PCR cycles, 10 μl of each reaction were loaded on a 1% agarose gel. As can be seen in FIG. 7, the band intensity for the CRISPR-cas9 cut samples is higher. It should be noted that in lane 3 the PCR smear is shifted down, we generally discard these types of products as the fraction of host-virus fragments is low. (A=unshared genomic DNA, B=genomic DNA sheared to 8 kb).

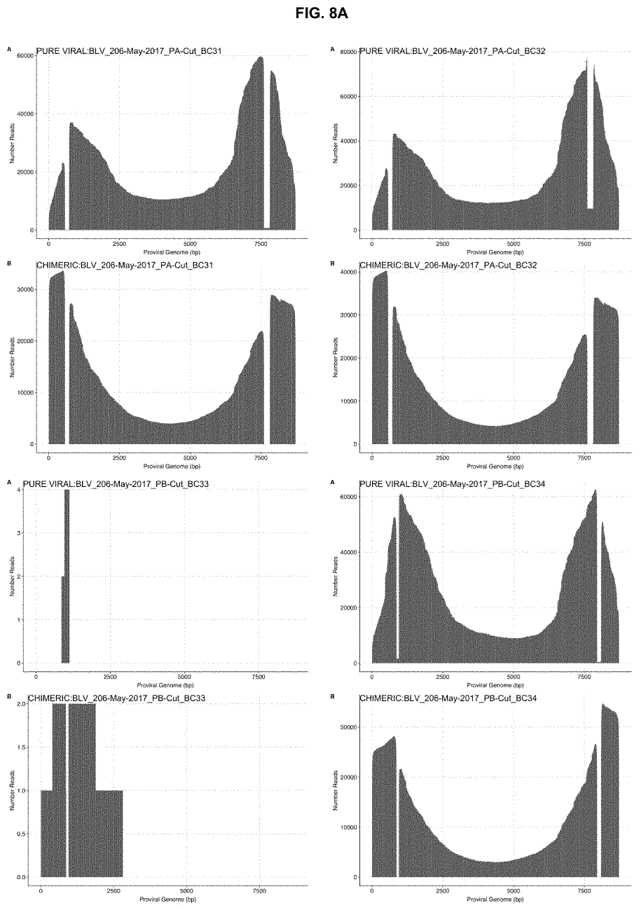

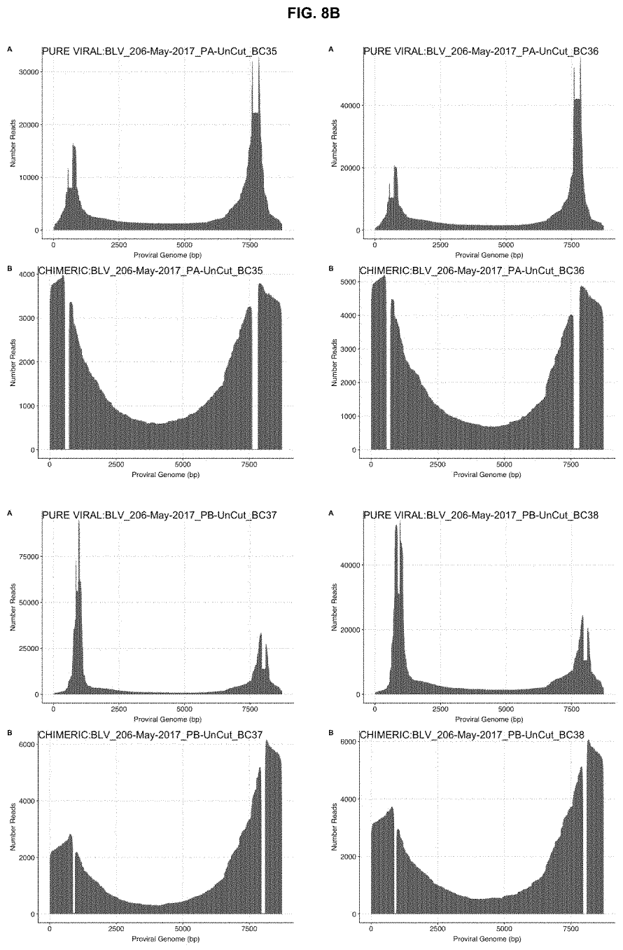

Following clean up and elution in ˜40 μl of H₂O we took an equal volume (3 μl) of each library and indexed them via PCR, in a 50 μl reaction volume and using 8 cycles. Again, following clean up, an equal volume of library was pooled and a nanopore library (LSK-109) was prepared and sequenced on a r9.4 flow cell. Base calling and demultiplexing was carried out as described in Example 1. The results are outlined in Table 9. In addition the coverage of the resultant reads is shown in FIGS. 8a and 8b.

TABLE 9

| | | DNA | | | | Pure | | | | | | | |
|---|---|---|---|---|---|---|---|---|---|---|---|---|---|
| | | concen- | DNA | | | Host/Pure | | | | | Largest | | Largest |
| | | tration | concen- | | Chimeric | Viral | | | | Insertion | clone | Insertion | clone |
| | | PCR 1 | trationPCR 2 | Raw | reads | reads | Mean | | Median | sites | PCIP | sites | Illumina |
| Lib | Treatment | (ng/ul) | (ng/ul) | reads | % | (%) | Length | N50 | Length | PCIP | (%) | Illumina | (%) |
| 1 | PA-Cut | 22.52 | 69.48 | 113,485 | 55.6 | 0.25/44.2 | 2880.6 | 3855.0 | 2217.0 | 2122 | 25.8 | 1700 | 30.849 |
| | BC31 | | | | | | | | | | | | |
| 2 | PA-Cut | 26.18 | 72.06 | 137,109 | 54.1 | 0.47/45.4 | 2770.6 | 3710.0 | 2141.0 | 2216 | 24.7 | ″ | ″ |
| | BC32 | | | | | | | | | | | | |
| 3 | PB-Cut | 71.85 | 63.7 | 6,844 | 1.01 | 98.5/0.51 | 263.8 | 277.0 | 195.5 | 2 | 50 | ″ | ″ |
| | BC33 | | | | | | | | | | | | |
| 4 | PB-Cut | 34.17 | 86.65 | 126,655 | 49.4 | 0.19/50.4 | 2616.2 | 3395.0 | 2010.0 | 2281 | 24.5 | ″ | ″ |
| | BC34 | | | | | | | | | | | | |
| 5 | PA-UnCut | 13.4 | 33.32 | 42,795 | 22.5 | 0.19/77.3 | 1759.8 | 2670.0 | 1227.0 | 660 | 30.9 | ″ | ″ |
| | BC35 | | | | | | | | | | | | |
| 6 | PA-UnCut | 17.26 | 42.53 | 66,602 | 19.7 | 0.19/80.2 | 1549.1 | 2381.0 | 1056.0 | 713 | 30.4 | ″ | ″ |
| | BC36 | | | | | | | | | | | | |
| 7 | PB-UnCut | 22.27 | 48.24 | 114,967 | 10.4 | 0.16/89.4 | 917.9 | 1579.0 | 497.0 | 690 | 29.5 | ″ | ″ |
| | BC37 | | | | | | | | | | | | |
| 8 | PB-UnCut | 14.71 | 35.92 | 64,789 | 18.1 | 0.19/81.7 | 1461.4 | 2111.0 | 992.0 | 736 | 30.4 | ″ | ″ |
| | BC38 | | | | | | | | | | | | |

Table 9 shows that libraries prepared with the CRISPR cut generally produced more raw reads and a much larger fraction of them is composed of the desired chimeric reads containing proviral and host DNA. The CRISPR cut libraries also identified a large number of integration sites. The comparison with an Illumina based library prepared from the same timepoint, using ˜4 ug of template, shows that PCIP can identify more integration sites. This experiment also shows that only libraries with a size distribution that mirrors that observed in the sheared DNA should be sequenced, libraries with a preponderance of shorter fragments mainly represent nonspecific amplification.

### Example 4: Identifying Genomic Insertions and Internal Variants in HTLV-1

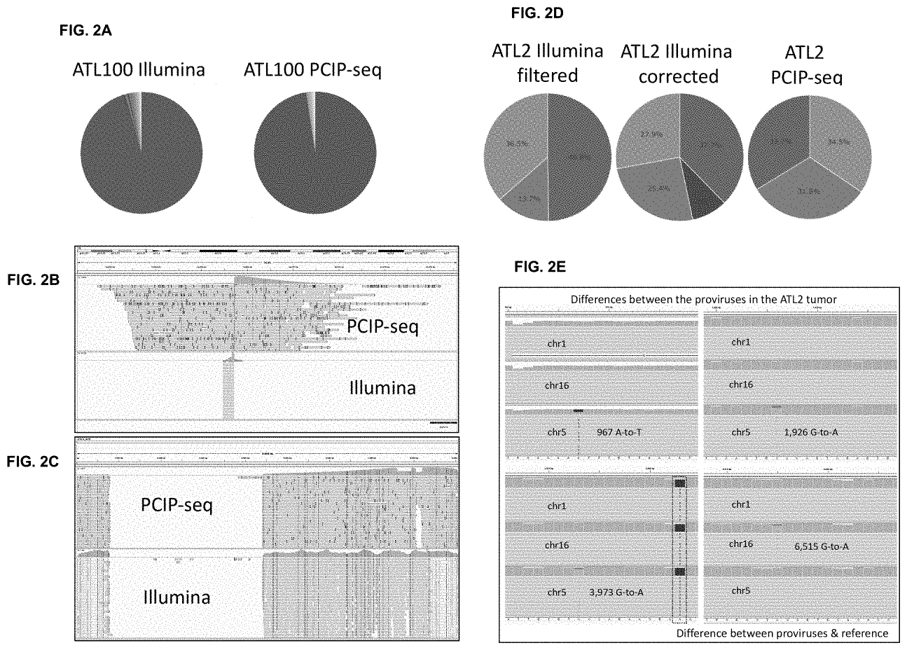

Adult T-cell leukemia (ATL) is an aggressive cancer induced by HTLV-1. It is generally characterized by the presence of a single dominant malignant clone, identifiable by a unique proviral integration site. We and others have developed methods based on ligation mediated PCR and Illumina sequencing to simultaneously identify integration sites and determine the abundance of the corresponding clones^{2.7}. We initially applied PCIP-seq to two HTLV-1 induced cases of ATL, both previously analyzed with our Illumina based method (ATL2⁷ & ATL100²⁰). In ATL100 both methods identify a single dominant clone, with >95% of the reads mapping to a single insertion site on chr18 (FIG. 2a, 2b & Table 8). Using the integration site information, we extracted the PCIP-seq hybrid reads spanning the provirus/host insertion site, uncovering a ˜3,600 bp deletion within the provirus (FIG. 2c).

In the case of ATL2, PCIP-seq showed three major proviruses located on chr5, chr16 and chr1, each responsible for ˜33% of the HTLV-1/host hybrid reads. We had previously established that these three proviruses are in a single clone via examination of the T-cell receptor gene rearrangement⁷. However, it is interesting to note that this was not initially obvious using our Illumina based method as the proviral insertion site on chr1 falls within a repetitive element (LTR) causing many of the reads to map to multiple regions in the genome. If multi mapping reads are filtered out, the chr1 insertion site accounted for 13.7% of the remaining reads, while retaining multi mapping produces values closer to reality (25.4%). In contrast the long reads from PCIP-seq allow unambiguous mapping and closely matched the expected 33% for each insertion site (FIG. 2d), highlighting the advantage long reads have in repetitive regions. Looking at the three proviruses, proviral reads revealed all to be full length. Three de novo mutations were observed in one provirus and a single de novo mutation was identified in the second (FIG. 2e).

### Example 5: Insertion Sites Identified in Samples with Multiple Clones of Low Abundance

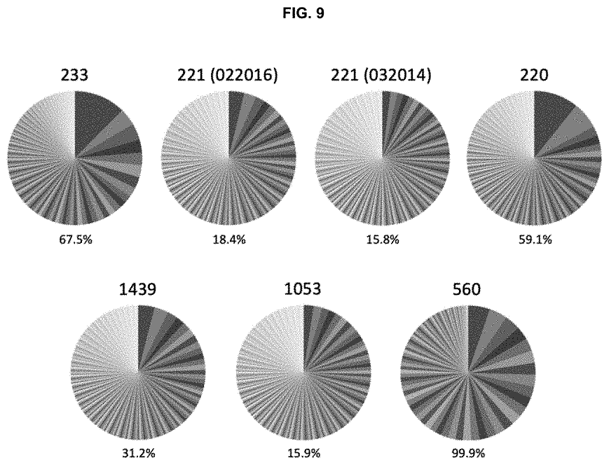

The samples utilized above represent a best-case scenario, with ˜100% of cells infected and a small number of major clones. We next applied PCIP-seq to four samples from BLV infected sheep (experimental infection²¹) and three cattle (natural infection) to explore its performance on polyclonal and low proviral load (PVL) samples and compared PCIP-seq to our previously published Illumina method⁷. PCIP-seq revealed all samples to be highly polyclonal (FIG. 9 and Table 8) with the number of unique insertion sites identified varying from 172 in the bovine sample 560 (1 μg template, PVL 0.644%) to 17,903 in bovine sample 1053 (6 μg template, PVL 23.5%). In general, PCIP-seq identified more insertion sites, using less input DNA than our Illumina based method (Table 10).

TABLE 10
Comparing PCIP-seq to ligation mediated PCR and Illumina sequencing. For the Illumina
libraries the template DNA used was 4 μg. For the PCIP-seq it varied between libraries (233 = 7 μg,
221(022016) = 4 μg, 221(032014) = 4 μg, 220 = 2 μg, 1439 = 3 μg, 560 = 1 μg,
1053 = 6 μg). >3 signifies insertion sites supported by more than 3 reads after PCR duplicate removal.
ILLUMINA = Ligation mediated PCR with Illumina sequencing. U-IS ILL. in PCIP = Unique insertion sites
(%) identified in ILLUMINA and also found in PCIP-seq. Correlation Abundance Overlapping IS. Pearson's
correlation Abundance = correlation of abundances from proviruses detected in both Illumina and PCIP-seq.

| | | | | | Insertion | Insertion | | | |
|---|---|---|---|---|---|---|---|---|---|
| | Insertion | Insertion | U-IS ILL. | | sites | sites | U-IS ILL. | Raw | Raw |
| | sites | sites | in PCIP | Pearson | ILLUMINA | PCIP-seq | in PCIP | PCIP-seq | Illumina |
| Sample | ILLUMINA | PCIP-seq | (%) | Correlation | (>3) | (>3) | (%) (>3) | reads | reads |
| 233 | 1110 | 5311 | 81.2 | 0.949810181 | 448 | 2302 | 85.9 | 524698 | 173196 |
| 221 (022016) | 1122 | 8023 | 40.4 | 0.511939213 | 74 | 3546 | 50 | 180276 | 9579 |
| 221 (032014) | 4473 | 5374 | 44.4 | 0.526457101 | 1555 | 1524 | 34.9 | 32266 | 391478 |
| 220 | 915 | 1352 | 36.1 | 0.894732877 | 401 | 664 | 47.6 | 44876 | 299554 |
| 1439 | 5784 | 5773 | 47.7 | 0.894732877 | 1449 | 3053 | 63.9 | 181055 | 216525 |
| 560 | 379 | 172 | 15.8 | 0.616804459 | 81 | 77 | 33.3 | 6802 | 192170 |
| 1053 | 8496 | 17903 | 62.0 | 0.811169919 | 2196 | 7777 | 68.5 | 367454 | 219461 |

Comparison of the results showed a significant overlap between the two methods. When we consider insertion sites supported by more than three reads in both methods (larger clones, more likely to be present in both samples), in the majority of cases >50% of the insertion sites identified in the Illumina data were also observed via PCIP-seq (Table 10). These results show the utility of PCIP-seq for insertion site identification, especially considering the advantages long reads have in repetitive regions of the genome.

### Example 6: Identifying SNPs in BLV Proviruses

Portions of the proviruses with more than ten supporting reads (PCR duplicates removed) were examined for SNPs with LoFreq²². For the four sheep samples, the variants were called relative to the pBLV344 provirus (used to infect the animals). For the bovine samples 1439 and 1053 custom consensus BLV sequences were generated for each and the variants were called in relation to the appropriate reference (SNPs were not called in 560). Across all the samples 3,209 proviruses were examined, 934 SNPs were called and 680 (21%) of the proviruses carried one or more SNPs (Table 11).

TABLE 11
Numbers of SNPs identified in each sample.

| | | | | | | | # |
|---|---|---|---|---|---|---|---|
| | | | | | | | Positions |
| | | | | # | # | # | within |
| | | | # | Proviruses | Variants | Proviruses | proviruses |
| Sample | | | Insertion | examined | detected | with variant | with variant |
| name | Species | PVL | sites | for SNPs | (AF > 0.6) | (AF > 0.6) | (AF > 0.6) |
| 233 | OAR | 78.3 | 5311 | 789 | 233 | 168 | 136 |
| 221 (022016) | OAR | 63.0 | 8023 | 408 | 93 | 79 | 86 |
| 221 (032014) | OAR | 16.0 | 5374 | 70 | 6 | 6 | 6 |
| 220 | OAR | 3.8 | 1352 | 130 | 50 | 42 | 36 |
| 1439 | BosT | 45.0 | 5773 | 587 | 311 | 211 | 137 |
| 1053 | BosT | 23.5 | 17903 | 1243 | 241 | 182 | 169 |

We validated 10 BLV SNPs in the ovine samples and 15 in the bovine via clone specific long-range PCR and Illumina sequencing. For Ovine 221, which was sequenced twice over a two-year interval, we identified and validated three instances where the same SNP and provirus were observed at both time points. We noted a small number of positions in the BLV provirus prone to erroneous SNP calls. By comparing allele frequencies from bulk Illumina and Nanopore data these problematic positions could be identified. For example, we observed a number of BLV proviruses in all the samples that had an apparent SNP at position 8213. When we looked at this position in reads mapped to the provirus without first sorting based on insertion site (referred to as bulk) we saw a C called 36 and 38% of the time respectively in the Nanopore data. In the bulk Illumina data, generated from the same sample, we saw the C is called 0% of the time indicating a technical artifact. As a consequence, SNPs from this position were excluded.

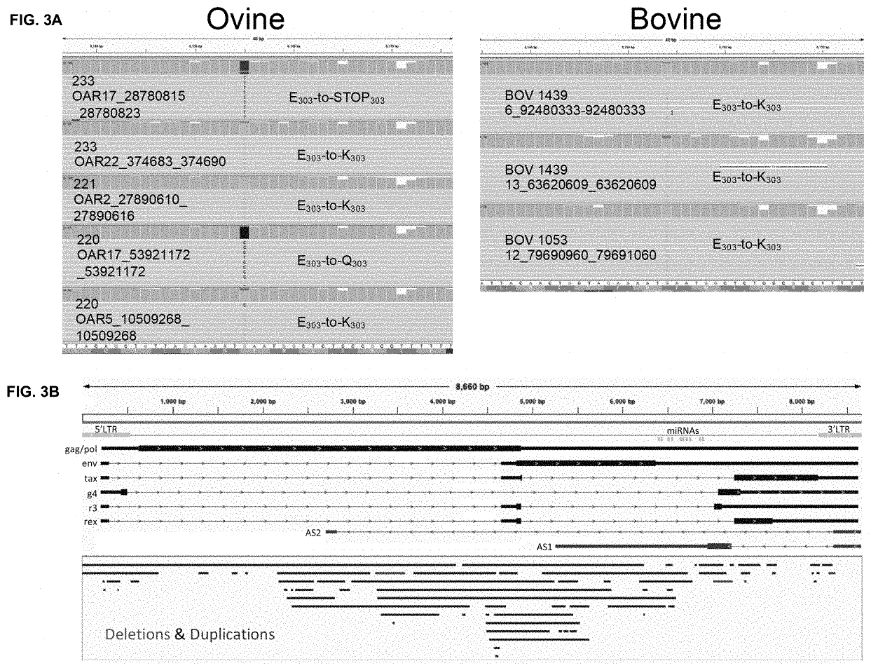

Approximately half of the SNPs (47.1% sheep, 51.6% cattle) were found in multiple proviruses. Generally, SNPs found at the same position in multiple proviruses were concentrated in a single individual, indicating their presence in a founder provirus or via a mutation in the very early rounds of viral replication. For example, in animal 233 we found 16 proviruses (provirus inclusion was based on the less stringent criteria of >10 reads covering the position, not filtered for PCR duplicates) carrying a T-to-C transition within the Tax ORF at position 8154, this variant does not change the amino acid. Illumina and Nanopore bulk sequencing from the same sample show C is called at a 2% frequency in Nanopore, while with Illumina C is called at a 1% frequency. This indicates that the SNPs observed in these proviruses are not a technical artifact. Alternatively, a variant may also rise in frequency due to increased fitness of clones carrying a mutation in that position. In this instance, we would expect to see the same position mutated in multiple individuals. One potential example is found in the first base of codon 303 (position 8155) of the viral protein Tax, a potent viral transactivator, stimulator of cellular proliferation and highly immunogenic²³. A variant was observed at this position in five proviruses for sheep 233 and three for sheep 221 as well as one provirus from bovine 1439 (FIG. 3a). Using less stringent criteria for the inclusion of a proviral region (>10 reads, not filtered for PCR duplicates) we found 34 proviruses in the ovine and 3 in the bovine carrying a variant in this position. The majority of the variants observed were G-to-A transitions (results in E-to-K amino acid change), however we also observed G-to-T (E-to-STOP) and G-to-C (E-to-Q) transversions. It has been previously shown that the G-to-A mutation abolishes the Tax proteins transactivator activity^{23,24}. The repeated selection of variants at this specific position suggests that they reduce viral protein recognition by the immune system, while preserving the Tax proteins other proliferative properties.

Patterns of provirus-wide APOBEC3G²⁵ induced hypermutation (G-to-A) were not observed in BLV. However, three proviruses (two from sheep 233 and one in bovine 1053) showed seven or more A-to-G transitions, confined to a ˜70 bp window in the first half of the U3 portion of the 3′LTR. The pattern of mutation, as well as their location in the provirus suggests the action of RNA adenosine deaminases 1 (ADAR1)^{26,27}.

### Example 7: PCIP-Seq Identifies BLV Structural Variants in Multiple Clones

Proviruses were also examined for structural variants (SVs) using a custom script and via visualization in IGV (see Example 1). Between the sheep and bovine samples, we identified 66 deletions and 3 tandem duplications, with sizes ranging from 15 bp to 4,152 bp, with a median of 113 bp (Table 12).

TABLE 12
BLV structural variants identified via PCIP-seq

| 1053 | | | | | 1439 | | | | |
|---|---|---|---|---|---|---|---|---|---|
| | | | | Clone | | | | | Clone |
| | | Region | Approx | specific | | | Region | Approx | specific |
| Provirus | Type | in BLV | size | PCR | Provirus | Type | in BLV | size | PCR |
| 1_120275095_120275095 | DEL | 230-252 | 22 | no | 10_65013091_65013093 | DEL | 2164-3192 | 1028 | no |
| 1_147862114_147862122 | DEL | 2241-2275 | 34 | no | 1_150385145_150385351 | DEL | 3451-3474 | 23 | yes |
| 2_106933456_106933462 | DEL | 7674-7708 | 34 | no | 2_121703720_121703726 | DEL | 5350-5399 | 49 | no |
| 3_6970332_6970339 | DEL | 5109-6728 | 1619 | no | 23_39892380_39892560 | DEL | 2364-2560 | 196 | yes |
| 3_90671155_90671163 | DEL | 2608-2919 | 311 | no | 2_4188067_4188067 | DEL | 2176-2570 | 394 | no |
| 4_114867583_114867589 | DEL | 4574-4637 | 63 | no | 24_3748146_3748155 | DEL | 5419-5497 | 78 | no |
| 5_25818093_25818100 | DEL | 4482-4526 | 44 | no | 27_36582809_36582809 | DEL | 4522-5636 | 1114 | yes |
| 6_95273607_95273614 | DEL | 4487-5537 | 1050 | no | 27_36582809_36582809 | DEL | 1-852 | 852 | yes |
| 6_112133285_112133291 | DEL | 5217-5368 | 151 | no | 4_100234239_100234246 | INS | 8296-8370 | 75 | yes |
| 10_101509344_101509352 | DEL | 7324-7425 | 101 | no | 5_51456241_51456285 | DEL | 1-4152 | 4152 | yes |
| 12_36183673_36183673 | DEL | 1808-1835 | 27 | no | 2_124084208_124084213 | DEL | 391-406 | 15 | yes |
| 13_35328779_35328785 | DEL | 3679-4603 | 924 | no | 3_45576532_45576538 | DEL | 2316-2336 | 20 | yes |
| 15_24605050_24605054 | DEL | 8136-8162 | 26 | no | 5_95348339_95348346 | DEL | 8167-8200 | 33 | no |
| 16_28380797_28380803 | DEL | 2984-3895 | 911 | no | 8_112613917_112613964 | DEL | 4225-6244 | 2019 | no |
| 17_64277037_64277043 | DEL | 5418-5636 | 218 | no | 5_6307451_6307451 | INS | 3251-3590 | 338 | no |
| 20_7882911_7882911 | DEL | 8111-8137 | 26 | no | | | | | |
| 20_7882911_7882911 | DEL | 8230-8340 | 110 | no | | | | | |
| 21_53434814_53434824 | DEL | 6854-7130 | 276 | no | | | | | |
| 21_53434814_53434824 | DEL | 7202-7246 | 44 | no | | | | | |
| 22_40343810_40343823 | DEL | 4629-4838 | 209 | no | | | | | |
| 22_48239823_48239830 | DEL | 2271-2799 | 528 | no | | | | | |
| 23_41760533_41760533 | DEL | 8100-8201 | 101 | no | | | | | |
| 24_22643966_22643974 | DEL | 6857-7165 | 308 | no | | | | | |
| 25_33749737_33749744 | DEL | 4225-4264 | 39 | no | | | | | |
| 28_28470239_28470248 | DEL | 4496-5191 | 695 | no | | | | | |
| 29_25146501_25146508 | DEL | 3901-5251 | 1350 | no | | | | | |
| X_33071616_33071616 | DEL | 3322-3969 | 647 | no | | | | | |
| X_61600607_61600612 | DEL | 6193-6783 | 590 | no | | | | | |

| 221 (022016 & 032014) | | | | | 221 (032014) | | | | |
|---|---|---|---|---|---|---|---|---|---|
| | | | | Clone | | | | | Clone |
| | | Region | Approx | specific | | | Region | Approx | specific |
| Provirus | Type | in BLV | size | PCR | Provirus | Type | in BLV | size | PCR |
| OAR3_128671913_128671921 | DEL | 4591-4620 | 30 | no | OAR14_25755878_25755884 | DEL | 5846-6486 | 640 | no |
| OAR18_26694984_26694991 | DEL | 5287-5508 | 222 | no | | | | | |
| OAR25_25097056_25097063 | DEL | 2325-4303 | 1979 | yes | | | | | |
| OARX_110727773_110727797 | DEL | 2858-2970 | 113 | no | | | | | |
| OARX_78143793_78143801 | DEL | 3284-6602 | 3298 | yes | | | | | |
| 221 (022016) | | | | | 233 | | | | |
| | | | | Clone | | | | | Clone |
| | | Region | Approx | specific | | | Region | Approx | specific |
| Provirus | Type | in BLV | size | PCR | Provirus | Type | in BLV | size | PCR |
| OAR1_25125478_25125485 | DEL | 6237-6255 | 19 | no | OAR10_34545991_34546003 | DEL | 5298-5330 | 32 | yes |
| OAR1_250672128_250672136 | DEL | 7365-7389 | 25 | yes | OAR10_49266255_49266262 | DEL | 6512-6586 | 74 | yes |
| OAR2_73878244_73878251 | DEL | 237-264 | 28 | no | OAR14_42146250_42146256 | DEL | 1658-1724 | 66 | no |
| OAR3_149619110_149619110 | DEL | 7610-7726 | 117 | no | OAR16_3998022_3998027 | DEL | 4479-4706 | 227 | no |
| OAR3_211678275_211678275 | DEL | 6228-6285 | 58 | no | OAR19_37466567_37466573 | DEL | 278-428 | 150 | no |
| OAR8_80161637_80161982 | DEL | 6502-6561 | 60 | yes | OAR23_14140808_14140814 | DEL | 3270-5878 | 2608 | no |
| OAR13_10090846_10090865 | DEL | 6484-6561 | 78 | no | OAR3_184106381_184106391 | DEL | 5799-5874 | 75 | no |
| OAR16_10037623_10037623 | DEL | 1287-1396 | 110 | no | OAR7_72584331_72584331 | DEL | 4574-5453 | 879 | no |
| OAR21_31148897_31148902 | DEL | 7292-7544 | 253 | no | OAR7_72649090_72649098 | DEL | 539-629 | 90 | no |
| OAR24_28280610_28280610 | DEL | 6807-6828 | 22 | no | | | | | |
| OAR2_242159705_242159712 | INS | 7017-7232 | 215 | yes | | | | | |

We validated 14 of these via clone specific PCR. As seen in FIG. 3b SVs were found throughout the majority of the provirus, encompassing the highly expressed microRNAs²⁸ as well as the second exon of the constitutively expressed antisense transcript AS1²⁹. Only two small regions at the 3′ end lacked any SVs. More proviruses will need to be examined to see if this pattern holds, but these results again suggest the importance of the 3′LTR and its previously reported interactions with adjacent host genes⁷.

### Example 8: Identifying HIV-1 Integration Sites and the Associated Provirus

Despite the effectiveness of combination antiretroviral therapy (ART) in suppressing HIV-1 replication, cART is not capable of eliminating latently infected cells, ensuring a viral rebound if cART is suspended³⁰. This HIV-1 reservoir represents a major obstacle to a HIV cure³¹ making its exploration a priority. However, this task is complicated by its elusiveness, with only ˜0.1% of CD4⁺ T cells carrying integrated HIV-1 DNA³². To see if PCIP-seq could be applied to these extremely low proviral loads we initially carried out dilution experiments using U1³³, a HIV-1 cell line containing replication competent proviruses³⁴. PCIP-seq on undiluted U1 DNA found the major insertion sites on chr2 and chrX (accounting for 47% & 41% of the hybrid reads respectively) and identified the previously reported variants that disrupt Tat function³⁵ in both proviruses. In the chr2 provirus a T-to-C changes ATG to ACG and the first methionine to a threonine. In the chrX provirus an A-to-T changes CAT to CTT replacing a histidine at position 13 with a leucine. In addition to the two major proviruses we identified an additional ˜700 low abundance insertion sites (Table 8) including one on chr19 (0.8%) reported by Symons et al 2017³⁴ that is actually a product of recombination between the major chrX and chr2 proviruses, and one on chr7 (chr7: 100.5). Identification of the chr7: 100.5 & chr19: 34.9 proviruses as the products of recombination between major chrX and chr2 proviruses was shown by mapping proviral reads from all four proviruses to a full length proviral genome (the sequence (SEQ ID NO:4) of the chrX provirus was used as the reference). This allowed to identify SNPs and sequences derived from respectively, the chr2 and chrX provides. We then serially diluted U1 DNA in Jurkat cell line DNA. PCIP-seq was carried out with 5 μg of template DNA where U1 represents 0.1% and 0.01% of the total DNA. We also processed 5 μg of Jurkat DNA in parallel as a negative control. The three PCIP-seq libraries were prepared using the same guides and primers. Following sequencing and demultiplexing the Jurkat negative control produced 12,137 reads, Jurkat+U1 0.01% produced 234,421 reads and Jurkat+U1 0.1% 252,913 reads. The resultant reads were mapped to the human genome. We were able to detect the major proviruses on chr2 and chrX in both dilutions (Table 8). The reads were also mapped the HIV-1 genome. No reads of pure HIV-1 or chimeric HIV-1/host reads mapping to HIV-1 were observed in the Jurkat negative control (Table 14). In Jurkat+U1 0.01% samples 12.6% of the reads were chimeric HIV-1/host, in Jurkat+U1 0.1% this rose to 43.2%.

### Example 9: Identifying Full-Length and Polymorphic Endogenous Retroviruses in Cattle and Sheep

ERVs in the genome can be present as full length, complete provirus, or more commonly as solo-LTRs, the products of non-allelic recombination³⁷. At the current time conventional short read sequencing, using targeted or whole genome approaches, cannot distinguish between the two classes. Examining full length ERVs would provide a more complete picture of ERV variation, while also revealing which elements can produce de novo ERV insertions. As PCIP-seq targets inside the provirus we can preferentially amplify full length ERVs, opening this type of ERV to study in larger numbers of individuals. As a proof of concept we targeted the class II bovine endogenous retrovirus BERVK2, known to be transcribed in the bovine placenta³⁸. We applied the technique to three cattle, of which one (10201e6) was a Holstein suffering from cholesterol deficiency, an autosomal recessive genetic defect recently ascribed to the insertion of a 1.3 kb LTR in the APOB gene³⁹. PCIP-seq clearly identified the APOB ERV insertion in 10201e6, whereas no reads were seen mapping to this position in libraries from the other two cattle (Mannequin & 571). In contrast to previous reports³⁹ PCIP-seq shows it to be a full-length element. We identified a total of 67 ERVs, with 8 present in all three samples (Table 15).

TABLE 15
Endogenous retroviruses (BERVK2) identified in cattle via PCIP-seq.

| # | Approximate location in genome (BTA6) | Provirus name | 10201e6 | Mannequin | 571 | Provirus |
|---|---|---|---|---|---|---|
| 1 | chr1: 108,822,892-108,832,262 | BTA1_108.8 | no | no | YES | Full |
| 2 | chr1: 140,473,236-140,486,732 | BTA1_140.4 | YES | no | YES | Full |
| 3 | chr2: 7,341,443-7,349,776 | BTA2_7.3 | no | no | YES | Full |
| 4 | chr2: 68,574,688-68,583,604 | BTA2_68.5 | YES | no | no | Partial |
| 5 | chr2: 108,763,340-108,771,071 | BTA2_108.7 | no | YES | no | Full |
| 6 | chr2: 136,856,893-136,860,100 | BTA2_136.8 | YES | no | no | Full |
| 7 | chr3: 11,025,879-11,032,187 | BTA3_11.0 | no | YES | no | Full |
| 8 | chr3: 21,243,379-21,247,173 | BTA3_21.24 | no | YES | no | Full |
| 9 | chr3: 21,262,507-21,266,148 | BTA3_21.26 | no | YES | no | Full |
| 10 | chr3: 115,305,677-115,313,191 | BTA3_115.3 | YES | no | no | Full* |
| 11 | chr4: 23,529,679-23,538,398 | BTA4_23.5 | YES | no | no | Partial |
| 12 | chr4: 106,804,424-106,812,368 | BTA4_106.8 | no | no | YES | Full |
| 13 | chr5: 76,505,040-76,518,833 | BTA5_76.5 | YES | YES | YES | Full |
| 14 | chr6: 19,795,982-19,804,772 | BTA6_19.7 | YES | YES | YES | Full |
| 15 | chr6: 33,664,998-33,674,349 | BTA6_33.6 | YES | no | no | Full |
| 16 | chr6: 93,979,584-93,984,028 | BTA6_93.9 | YES | YES | YES | Partial |
| 17 | chr7: 18,507,208-18,514,234 | BTA7_18.5 | no | YES | no | Partial |
| 18 | chr7: 62,318,935-62,329,558 | BTA7_62.3 | YES | no | no | Full |
| 19 | chr7: 109,501,965-109,512,061 | BTA7_109.5 | YES | no | YES | Full |
| 20 | chr8: 16,410,224-16,424,259 | BTA8_16.4 | YES | no | YES | Full |
| 21 | chr8: 37,357,029-37,369,016 | BTA8_37.3 | no | YES | no | Full |
| 22 | chr8: 67,963,331-67,972,754 | BTA8_67.9 | no | YES | no | Full |
| 23 | chr8: 81,237,785-81,244,766 | BTA8_81.2 | YES | YES | no | Full |
| 24 | chr9: 15,412,806-15,418,477 | BTA9_15.4 | YES | no | no | Partial |
| 25 | chr9: 83,082,008-83,092,749 | BTA9_83.0 | YES | no | no | Full |
| 26 | chr9: 84,257,434-84,262,548 | BTA9_84.2 | YES | no | no | Full |
| 27 | chr9: 101,949,614-101,957,434 | BTA9_101.9 | YES | YES | no | Full |
| 28 | chr10: 71,920,524-71,928,975 | BTA10_71.9 | YES | no | no | Full |
| 29 | chr10: 87,425,735-87,443,841 | BTA10_87.4 | YES | YES | YES | Partial |
| 30 | chr11: 50,592,847-50,606,524 | BTA11_50.5 | YES | no | YES | Full |
| 31 | chr11: 61,788,705-61,792,024 | BTA11_61.7 | no | YES | no | Full |
| 32 | chr11: 77,955,413-77,963,724 | BTA11_77.9 | YES | no | no | Full# |
| 33 | chr12: 72,978,039-72,985,406 | BTA12_72.9 | YES | YES | no | Full |
| 34 | chr12: 74,723,248-74,731,915 | BTA12_74.7 | YES | YES | no | Partial |
| 35 | chr15: 9,435,764-9,439,369 | BTA15_9.4 | YES | YES | YES | Full |
| 36 | chr16: 10,720,162-10,727,571 | BTA16_10.7 | YES | no | no | Full |
| 37 | chr16: 13,308,596-13,315,659 | BTA16_13.3 | YES | no | no | Partial |
| 38 | chr16: 28,504,653-28,536,456 | BTA16_28.5 | YES | no | YES | Full |
| 39 | chr18: 27,619,893-27,626,348 | BTA18_27.6 | YES | no | YES | Partial |
| 40 | chr18: 27,715,161-27,722,285 | BTA18_27.7 | no | no | YES | Full |
| 41 | chr18: 50,368,602-50,378,304 | BTA18_50.3 | YES | YES | YES | Full |
| 42 | chr18: 60,211,168-60,220,590 | BTA18_60.2 | YES | YES | YES | Partial |
| 43 | chr18: 61,691,367-61,697,347 | BTA18_61.6 | YES | no | YES | Full |
| 44 | chr19: 5,180,841-5,189,334 | BTA19_5.1 | YES | no | no | Partial |
| 45 | chr19: 22,014,748-22,025,138 | BTA19_22.0 | YES | no | no | Full |
| 46 | chr19: 51,039,969-51,101,363 | BTA19_51.0 | no | YES | YES | Partial |
| 47 | chr20: 15,283,426-15,290,599 | BTA20_15.2 | YES | no | no | Full |
| 48 | chr20: 55,126,259-55,134,120 | BTA20_55.1 | no | YES | no | Full |
| 49 | chr21: 1,241,740-1,256,399 | BTA21_1.2 | YES | YES | YES | Partial |
| 50 | chr21: 2,303,211-2,307,834 | BTA21_2.3 | no | YES | no | Full |
| 51 | chr21: 4,133,180-4,142,631 | BTA21_4.1 | no | no | no | Full |
| 52 | chr21: 18,634,068-18,645,042 | BTA21_18.6 | no | YES | no | Full |
| 53 | chr22: 160,456-166,792 | BTA22_160.4 | no | no | YES | Full |
| 54 | chr23: 41,312,657-41,328,100 | BTA23_41.3 | YES | no | no | Full |
| 55 | chr23: 52,329,640-52,337,577 | BTA23_52.3 | YES | no | no | Full |
| 56 | chr24: 12,819,683-12,824,449 | BTA24_12.6 | YES | YES | no | Partial |
| 57 | chr24: 53,067,680-53,078,844 | BTA24_53.0 | no | no | YES | Full |
| 58 | chr25: 20,428,960-20,444,963 | BTA25_20.4 | no | no | no | Full |
| 59 | chr26: 50,606,858-50,616,960 | BTA26_50.6 | YES | no | no | Full |
| 60 | chr27: 14,146,146-14,156,627 | BTA27_14.1 | no | YES | no | Full |
| 61 | chr28: 17,575,320-17,582,731 | BTA28_17.5 | YES | no | no | Full |
| 62 | chr29: 39,631,808-39,639,476 | BTA29_39.6 | YES | no | no | Full |
| 63 | chrX: 27,723,875-27,732,458 | BTAX_27.7 | no | YES | no | Full |
| 64 | chrX: 30,183,463-30,187,122 | BTAX_30.1 | YES | no | no | Partial |
| 65 | chrX: 36,260,818-36,264,888 | BTAX_36.2 | YES | no | no | Partial |
| 66 | chrX: 43,949,278-43,960,449 | BTAX_43.9 | no | no | YES | Full |
| 67 | chrX:47,314,044-47,327,526 | BTAX_47.3 | no | no | YES | Full |

*LTR matches APOB ERV (BTA11_77.9);
#ERV inserted into APOB; Full = Full length ERV; Partial = ERV with large deletion.

We validated three ERVs via long range PCR and Illumina sequencing. We did not find any with an identical sequence to the APOB ERV, although the ERV BTA3_115.3 has an identical LTR sequence, highlighting that the sequence of the LTR cannot be used to infer the complete sequence of the ERV.

We also adapted PCIP-seq to amplify the Ovine endogenous retrovirus Jaagsiekte sheep retrovirus (enJSRV), a model for retrovirus-host co-evolution⁴⁰. The PCIP-seq reads were mapped to the reference genome (OAR3) where sequences matching enJSRV had been masked out, this preventing reads from multiple proviruses mapping to these positions. Hybrid reads in the unique flanking sequence allowed us to determine the sequence of the proviruses present at these locations. Using two sheep (220 & 221) as template we identified a total of 48 enJSRV proviruses, (33 in 220 and 38 in 221, with 22 common to both) and of these ˜54% were full length (Table 16).

TABLE 16
Endogenous retroviruses (enJSRV) identified in sheep via PCIP-seq.
Full = Full length ERV; Partial = ERV with large deletion.

| | Approximate | | | | pro- |
|---|---|---|---|---|---|
| | location in genome (OAR3) | ERV name | 220 | 221 | virus |
| 1 | chr1: 57,132,178-57,139,903 | OAR1_57.13 | no | YES | Full |
| 2 | chr1: 86,065,652-86,091,348 | OAR1_86.0 | YES | YES | Full |
| 3 | chr1: 129,489,883-129,502,056 | OAR1_129.4 | no | YES | Full |
| 4 | chr1: 220,250,002-220,258,800 | OARl_220.2 | YES | YES | Full |
| 5 | chr1: 240,077,458-240,092,905 | OAR1_240.0 | YES | YES | Partial |
| 6 | chr1: 253,739,233-253,756,582 | OAR1_253.7 | YES | YES | Partial |
| 7 | chr2: 196,585,537-196,593,010 | OAR2_196.5 | YES | no | Full |
| 8 | chr3: 39,261,134-39,285,428 | OAR3_39.2 | YES | YES | Full |
| 9 | chr3: 39653898-39656987 | OAR3_39.6 | YES | YES | Partial |
| 10 | chr3: 151,767,643-151,783,037 | OAR3_151.7 | YES | YES | Partial |
| 11 | chr3: 182,538,937-182,555,692 | OAR3_182.5 | YES | no | Full |
| 12 | chr4: 40,485,410-40,504,790 | OAR4_40.4 | YES | YES | Full |
| 13 | chr4: 77,416,611-77,428,510 | OAR4_77.4 | YES | YES | Partial |
| 14 | chr5: 7,744,521-7,756,178 | OAR5_7.74 | YES | YES | Partial |
| 15 | chr5: 64,916,815-64,926,920 | OAR5_64.9 | YES | no | Partial |
| 16 | chr5: 73,009,027-73,018,771 | OAR5_73.0 | YES | no | Full |
| 17 | chr6: 5,400,881-5,410,594 | OAR6_5.4 | no | YES | Full |
| 18 | chr6: 6,789,991-6,858,767 | OAR6_6.7 | YES | YES | Partial |
| 19 | chr6: 26,968,086-26,977,558 | OAR6_26.9 | no | YES | Full |
| 20 | chr8: 2,974,531-2,988,179 | OAR8_2.9 | YES | YES | Partial |
| 21 | chr8: 49,483,598-49,499,241 | OAR8_49.4 | YES | YES | Partial |
| 22 | chr9: 48,096,442-48,105,912 | OAR9_48.0 | no | YES | Full |
| 23 | chr9: 89,743,769-89,752,495 | OAR9_89.7 | no | YES | Partial |
| 24 | chr10: 70,892,072-70,919,960 | OAR10_70.8 | YES | no | Partial |
| 25 | chr11: 32,085,050-32,095,786 | OAR11_32.0 | YES | YES | Full |
| 26 | chr13: 5,676,353-5,686,765 | OAR13_5.6 | no | YES | Full |
| 27 | chr13: 16,714,529-16,726,069 | OAR13_16.7 | YES | YES | Full |
| 28 | chr13: 37,514,438-37,529,955 | OAR13_37.5 | YES | YES | Full |
| 29 | chr13: 66022872-66031772 | OAR13_66.0 | YES | no | Full |
| 30 | chr14: 13,811,039-13,844,103 | OAR14_13.8 | YES | YES | Partial |
| 31 | chr14: 15,011,370-15,043,076 | OAR14_15.0 | YES | YES | Partial |
| 32 | chr14: 56,232,971-56,236,157 | OAR14_56.2 | YES | YES | Full |
| 33 | chr14: 57,491,683-57,503,056 | OAR14_57.4 | no | YES | Partial |
| 34 | chr14: 57,605,121-57,623,737 | OAR14_57.6 | YES | YES | Partial |
| 35 | chr15: 10,864,017-10,870,430 | OAR15_10.8 | no | YES | Full |
| 36 | chr17: 48,876,178-48,887,208 | OAR17_48.8 | no | YES | Full |
| 37 | chr18: 1,738,143-1,751,356 | OAR18_1.7 | no | YES | Partial |
| 38 | chr18: 67,778,281-67,799,930 | OAR18_67.7 | YES | YES | Full |
| 39 | chr19: 52,665,989-52,689,785 | OAR19_52.6 | YES | YES | Partial |
| 40 | chr20: 433,819-443,901 | OAR20_0.4 | YES | no | Full |
| 41 | chr20: l,237,366-1,250,699 | OAR20_1.2 | no | YES | Partial |
| 42 | chr20: 27,598,593-27,615,677 | OAR20_27.5 | no | YES | Full |
| 43 | chr21: 6,694,384-6,709,701 | OAR21_6.6 | YES | no | Partial |
| 44 | chr22: 46,781,990-46,790,196 | OAR22_46.7 | no | YES | Full |
| 45 | chr26: 8,253,764-8,265,010 | OAR26_8.2 | no | YES | Full |
| 46 | chrX: 3,690,949-3,701,009 | OARX_3.6 | YES | no | Full |
| 47 | chrX: 62,939,566-62,949,333 | OARX_62.9 | YES | YES | Partial |
| 48 | chrX: 78,127,416-78,132,398 | OARX_78.1 | YES | no | Partial |

We validated seven proviruses via long-range PCR and Illumina sequencing.

### Example 10: Extending PCIP-Seq to Human Papillomaviruses (HPV)

The majority of HPV infections clear or are suppressed within 1-2 years⁴¹, however a minority evolve into cancer, and these are generally associated with integration of the virus into the host genome. This integration into the host genome is not part of the viral lifecycle and the breakpoint in the viral genome can occur at any point across is 8 kb circular genome¹⁶. As a consequence the part of the viral genome found at the virus host breakpoint varies considerably, making the identifying of integration sites difficult using existing approaches¹⁶. The long reads employed by PCIP-seq mean that even when the breakpoint is a number of kb away from the position targeted by primers we should still capture the integration site. As a proof of concept, we applied PCIP-seq to two HPV18 positive cases, (HPV18_PX and HPV18_PY) using 4 μg of DNA extracted from Pap smear material. We identified 55 integration sites in HPV18_PX and 19 integration sites in HPV18_PY (Table 17).

TABLE 17
HPV integration sites identified in patients HPV18_PX and HPV18_PY.
Estimated read count refers to number of reads after PCR duplicates have been
removed, see https://github.com/GIGA-AnimalGenomics-
BLV/PCIP/blob/master/README.md

| | | Estimated | | | |
|---|---|---|---|---|---|
| | | read | Overlapping | | |
| Patient | ID | count | Gene | geneID | Notes |
| HPV18_PX | chr1: 201993711-201993711 | 1 | RNPEP | ENSG00000176393 | |
| HPV18_PX | chr1: 54070808-54070808 | 1 | TCEANC2 | ENSG00000116205 | |
| HPV18_PX | chr1: 74339164-74339164 | 2 | FPGT-TNNI3K | ENSG00000259030 | |
| HPV18_PX | chr11: 72988358-72988358 | 6 | FCHSD2 | ENSG00000137478 | |
| HPV18_PX | chr12: 124528897-124528897 | 5 | NCOR2 | ENSG00000196498 | |
| HPV18_PX | chr12: 62430096-62430096 | 3 | NA | NA | |
| HPV18_PX | chr12: 88750111-88750111 | 2 | NA | NA | |
| HPV18_PX | chr13: 32401471-32401471 | 1 | N4BP2L1 | ENSG00000139597 | |
| HPV18_PX | chr13: 59883976-59883976 | 1 | DIAPH3 | ENSG00000139734 | |
| HPV18_PX | chr13: 70017637-70017637 | 1 | KLHL1 | ENSG00000150361 | |
| HPV18_PX | chr13: 96145444-96145444 | 1 | HS6ST3 | ENSG00000185352 | |
| HPV18_PX | chr16: 35696743-35696743 | 4 | NA | NA | |
| HPV18_PX | chr16: 46391666-46391666 | 15 | NA | NA | |
| HPV18_PX | chr16: 60839237-60839237 | 3 | NA | NA | |
| HPV18_PX | chr17: 50736162-50736162 | 1 | LUC7L3 | ENSG00000108848 | |
| HPV18_PX | chr17: 71945217-71945217 | 1 | NA | NA | |
| HPV18_PX | chr18: 33256597-33256597 | 2 | CCDC178 | ENSG00000166960 | |
| HPV18_PX | chr2: 175176252-175176252 | 1 | NA | NA | |
| HPV18_PX | chr2: 184979785-184979785 | 1 | NA | NA | |
| HPV18_PX | chr2: 222973976-222973976 | 1 | NA | NA | |
| HPV18_PX | chr20: 26724089-27697774 | 1 | NA | NA | Virus in satellite |
| | | | | | repeat |
| HPV18_PX | chr20: 59882951-59882951 | 4 | SYCP2 | ENSG00000196074 | |
| HPV18_PX | chr21: 31443081-31443081 | 5 | TIAM1 | ENSG00000156299 | |
| HPV18_PX | chr21: 8210410-8210516 | 6 | FP671120.3 | ENSG00000280800 | |
| HPV18_PX | chr21: 8225927-8228889 | 9 | FP671120.1 | ENSG00000278996 | |
| HPV18_PX | chr21: 8393406-8393551 | 9 | FP236383.2 | ENSG00000280614 | |
| HPV18_PX | chr21: 8437761-8437761 | 9 | FP236383.3 | ENSG00000281181 | |
| HPV18_PX | chr21: 8453856-8454775 | 19 | NA | NA | |
| HPV18_PX | chr3: 141177260-141177260 | 1 | NA | NA | |
| HPV18_PX | chr3: 183646815-183646815 | 5 | KLHL24 | ENSG00000114796 | |
| HPV18_PX | chr3: 52477576-52477615 | 67 | NISCH | ENSG00000010322 | |
| HPV18_PX | chr3: 52491989-52492028 | 67 | NISCH | ENSG00000010322 | |
| HPV18_PX | chr3: 52564151-52564190 | 75 | SMIM4 | ENSG00000168273 | |
| HPV18_PX | chr4: 113196089-113196089 | 3 | ANK2 | ENSG00000145362 | |
| HPV18_ PX | chr4: 118149173-118149173 | 2 | NDST3 | ENSG00000164100 | |
| HPV18_PX | chr4: 125160196-125160196 | 2 | NA | NA | |
| HPV18_PX | chr4: 8361851-8361851 | 1 | NA | NA | |
| HPV18_PX | chr5: 85159333-85159333 | 2 | NA | NA | |
| HPV18_PX | chr6: 12217019-12217019 | 1 | NA | NA | |
| HPV18_PX | chr6: 58604926-59721758 | 1 | NA | NA | Virus in satellite |
| | | | | | repeat |
| HPV18_PX | chr6: 60995120-60995120 | 4 | NA | NA | |
| HPV18_PX | chr6: 72218404-72218404 | 3 | RIMS1 | ENSG00000079841 | |
| HPV18_PX | chr6: 7655460-7655460 | 6 | NA | NA | |
| HPV18_PX | chr7: 55353950-55353950 | 10 | NA | NA | |
| HPV18_PX | chr7: 63798384-63798384 | 3 | NA | NA | |
| HPV18_PX | chr7: 7812181-7812181 | 4 | AC007161.3 | ENSG00000283549 | |
| HPV18_PX | chr7: 98111088-98111088 | 1 | LMTK2 | ENSG00000164715 | |
| HPV18_PX | chr8: 119801685-119801685 | 13 | TAF2 | ENSG00000064313 | |
| HPV18_PX | chr8: 2564068-2564068 | 1 | NA | NA | |
| HPV18_PX | chr8: 93515097-93515097 | 1 | LINC00535 | ENSG00000246662 | |
| HPV18_PX | chr8: 9886409-9886409 | 2 | NA | NA | |
| HPV18_PX | chr9: 12503146-12503146 | 1 | NA | NA | |
| HPV18_PX | chr9: 128458663-128458663 | 1 | ODF2 | ENSG00000136811 | |
| HPV18_PX | chrX: 19414286-19414286 | 1 | MAP3K15 | ENSG00000180815 | |
| HPV18_PX | chrX: 41675298-41675299 | 1 | CASK | ENSG00000147044 | |
| HPV18_PY | chr5: 37774016-37774016 | 2 | NA | NA | |
| HPV18_PY | chr7: 64329003-64329003 | 2 | ZNF736 | ENSG00000234444 | |
| HPV18_PY | chr4: 184039889-184039889 | 2 | NA | NA | |
| HPV18_PY | chr18: 108534-108534 | 2 | NA | NA | |
| HPV18_PY | chr3: 59699600-59699600 | 1 | NA | NA | |
| HPV18_PY | chr4: 90546531-90546531 | 1 | CCSER1 | ENSG00000184305 | |
| HPV18_PY | chr5: 146985347-146985347 | 1 | PPP2R2B | ENSG00000156475 | |
| HPV18_PY | chr6: 41200232-41200232 | 1 | TREML2 | ENSG00000112195 | |
| HPV18_PY | chr6: 113561576-113561576 | 1 | NA | NA | |
| HPV18_PY | chr1: 107169512-107169512 | 1 | NTNG1 | ENSG00000162631 | |
| HPV18_PY | chr1: 218361256-218361256 | 1 | TGFB2 | ENSG00000092969 | |
| HPV18_PY | chr3: 52563123-52563123 | 1 | SMIM4 | ENSG00000168273 | |
| HPV18_PY | chr9: 15686595-15686595 | 1 | CCDC171 | ENSG00000164989 | |
| HPV18_PY | chr9: 137787856-137787856 | 1 | AL590627.1 | ENSG00000255585 | |
| HPV18_PY | chr10: 6703026-6703026 | 1 | AL158210.2 | ENSG00000285743 | |
| HPV18_PY | chr10: 23788794-23788794 | 1 | KIAA1217 | ENSG00000120549 | |
| HPV18_PY | chr10: 91570894-91570894 | 1 | NA | NA | |
| HPV18_PY | chr11: 97096506-97096506 | 1 | NA | NA | |
| HPV18_PY | chr19: 35339090-35339090 | 1 | CD22 | ENSG00000012124 | |

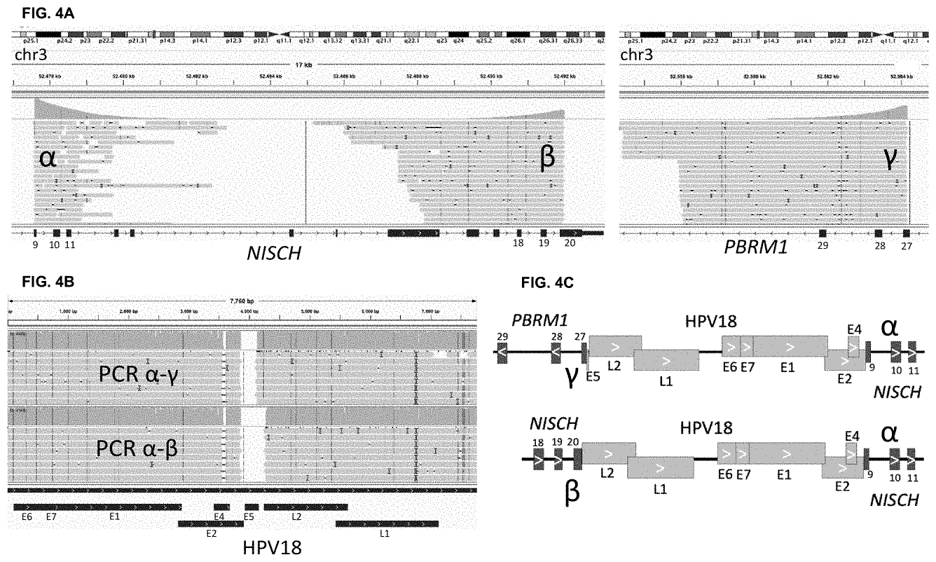

In HPV18_PY the vast majority of the reads only contained HPV sequences, the integration sites identified were defined by single reads, suggesting little or no clonal expansion (Table 8). In HPV18_PX most integration sites were again defined by a single read, however there were some exceptions (Table 17). HPV18_PX had integrated copies of HPV18 on chr21 and chr3 (FIGS. 4a-4c). Both integration sites contained multiple copies of the HPV genome. The most striking of these was a cluster of what appeared to be three integration sites located within the region chr3:52477576-52564190 (FIG. 4a). The unusual pattern of read coverage combined with the close proximity of the virus-host breakpoints indicated that these three integration sites were connected. Long range

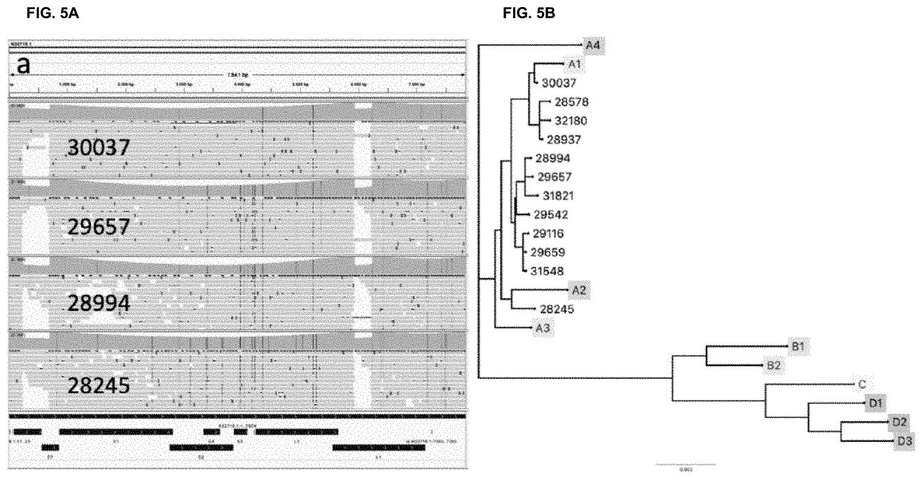

PCR with primers spanning positions α-β and α-γ, showed that a genomic rearrangement had occurred in this clonally expanded cell (FIG. 4a). Regions α and β are adjacent to one another with HPV integrated between, however PCR also showed regions α and γ to be adjacent to one another, again with the HPV genome integrated between (FIG. 4b). The sequence of the virus found between α-β looks to be derived from the α-γ virus as it shares a breakpoint and is slightly shorter (FIG. 4b). This complex arrangement suggests that this rearrangement was generated via the recently described ‘looping’ integration mechanism^{16,42}. The α and β breakpoints fall within exons of the NISCH gene while the γ breakpoint falls within exon 27 of PBRM1 (FIG. 4c), a gene previously shown to be a cancer driver in renal carcinoma⁴³ and intrahepatic cholangiocarcinomas⁴⁴. This patient was classified by histology as having atypical glandular cells and a follow up three months later was classified as a high grade CIN3. The PCIP-seq method was applied to DNA from leftover Pap smears, assaying 29 HPV18 and 42 HPV16 positive cases. The majority of the samples had been classified by cytology as Atypical squamous cells of undetermined significance (ASC-US). In both, episomal HPV was the most common finding. We found that the reads generated from episomal HPV can be used to generate a consensus sequence for HPV and as shown in FIGS. 5a and 5b it is possible to examine the phylogenetic relationships between the isolates.

As regards HPV integrations, we identified six patients where integration is associated with a pronounced clonal expansion, four, including HPV18_PX, were infected with HPV18 and two with HPV16.

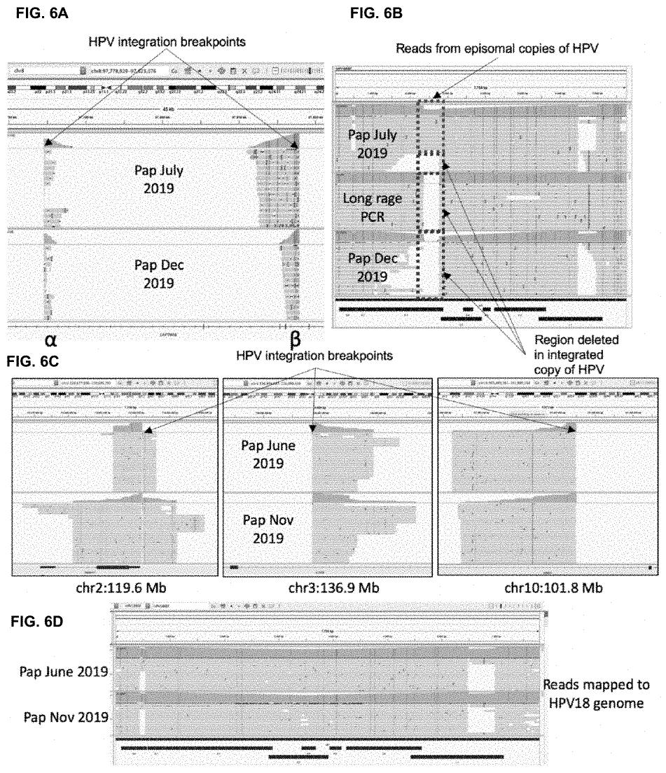

The second patient had an integration of HPV18 within an intron of LRRC49 (histology=low grade squamous intraepithelial lesion). From the next two clonally expanded integrations (both HPV18), samples from two time points were available. The first had an integration in the LAPTM4B gene, the integration was found in both samplings and in the second it appears that episomal HPV18 has been cleared (FIGS. 6a and 6b). (Histology, 1st sample=atypical squamous cells cannot exclude HSIL, 2nd sampling upgraded to High Grade Squamous Intraepithelial Lesion, HSIL).

The last clonally expanded integrations were found in a seventy-one-year-old patient, integration was observed in three different positions in the genome, all were observed in two samplings 5 months apart (FIGS. 6c and 6d) (Both time points, histology=atypical squamous cell of undetermined significance). All the integrated copies of HPV18 had intact E6 and E7 genes (both are cancer driver genes and are deregulated when HPV integrates).

As regards HPV16, we identified two samples with clonally expanded integrations. The first was observed in a 53-year-old with a low-grade squamous intraepithelial lesion, the HPV16 genome had integrated ˜2.5 kb upstream of the KRT5 gene. No episomal HPV16 DNA was observed in this sample. The integrated HPV genome contains a ˜3 kb deletion that does not overlap with the E6 and E7 genes. The second HPV16 sample has an integration in intron 4 of the POFUT1 gene. Again, the inserted viral genome contains a large deletion (˜5.5 kb) that does not overlap with E6 and E7. In contrast to the other HPV16 sample the majority (˜75%) of the HPV16 reads in this patient were still derived from episomal HPV16.

Discussion

In the present report we describe how PCIP-seq can be utilized to identify insertion sites while also sequencing parts of, and in some cases the entire associated provirus, and confirm this methodology is effective with a number of different retroviruses as well as HPV. For insertion site identification, the method was capable of identifying more than ten thousand BLV insertion sites in a single sample, using ˜4 μg of template DNA. Even in samples with a PVL of 0.66%, it was possible to identify hundreds of insertion sites with only 1 μg of DNA as template. The improved performance of PCIP-seq in repetitive regions further highlights its utility, strictly from the standpoint of insertion site identification. In addition to its application in research, high throughput sequencing of retrovirus insertion sites has shown promise as a clinical tool to monitor ATL progression²⁰. Illumina based techniques require access to a number of capital-intensive instruments. In contrast PCIP-seq libraries can be generated, sequenced and analyzed with the basics found in most molecular biology labs, moreover, preliminary results are available just minutes after sequencing begins⁴⁵. As a consequence, the method may have use in a clinical context to track clonal evolutions in HTLV-1 infected individuals, especially as the majority of HTLV-1 infected individuals live in regions of the world with poor biomedical infrastructure.

One of the common issues raised regarding Oxford Nanopore data is read accuracy. Early versions of the MinION had read identities of less than 60%⁴⁷, however the development of new pores and base calling algorithms make read identities of ˜90% achievable. Accuracy can be further improved by generating a consensus from multiple reads, making accuracies of ˜99.4%⁴⁸ possible. Recently Greig et al⁴⁹ compared the performance of Illumina and Oxford Nanopore technologies for SNP identification in two isolates of Escherichia coli. They found that after accounting for variants observed at 5-methylcytosine motif sequences only ˜7 discrepancies remained between the platforms. It should be noted that as PCIP-seq sequences PCR amplified DNA, errors generated by base modifications will be avoided. Despite these improvements in accuracy, Nanopore specific errors can be an issue at some positions. Comparison with Illumina data is helpful in the identification of problematic regions and custom base calling models may be a way to improve accuracy in such regions⁴⁸. Additionally, PCIP-seq libraries could equally be sequenced using long reads on the Pacific Biosciences platform or via 10× Genomics linked reads on Illumina if high single molecule accuracy is required¹⁷. In the current study we focused on SNPs observed in clonally expanded BLV proviruses. For viruses such as HIV-1, which have much lower proviral loads, more caution will be requited as the majority of proviral sequences will be generated from single provirus, making errors introduced by PCR more of an issue.

When analyzing SNPs from BLV the most striking result was the presence of the recurrent mutations at the first base of codon 303 in the viral protein Tax, a central player in the biology of both HTLV-1⁴⁶ and BLV⁵⁰. It has previously been reported that this mutation causes an E-to-K amino acid substitution which ablates the transactivator activity of the Tax protein²³. Collectively, these observations suggest this mutation confers an advantage to clones carrying it, possibly contributing to immune evasion, while retaining Tax protein functions that contribute to clonal expansion. However, there is a cost to the virus as this mutation prevents infection of new cells due to the loss of Tax mediated transactivation of the proviral 5′LTR making it an evolutionary dead end. It will be interesting to see if PCIP-seq can provide a tool to identify other examples of variants that increase the fitness of the provirus in the context of an infected individual but hinder viral spread to new hosts. Additionally, the technique could be used to explore the demographic features of the proviral population within and between hosts, how these populations evolve over time and how they vary.

A second notable observation is the cluster of A-to-G transitions observed within a ˜70 bp window in the 3′LTR. Similar patterns have been ascribed to ADAR1 hypermutation in a number of viruses²⁶, including the close BLV relatives HTLV-2 and simian T-cell leukemia virus type 3 (STLV-3)⁵¹. Given the small number of hypermutated proviruses observed, it appears to be a minor source of variation in BLV, although it will be interesting to see it this holds for different retroviruses and at different time points during infection.

In the current study we focused our analysis on retroviruses and ERVs. However, as this methodology is potentially applicable to a number of different targets we extended its use to HPV as a proof of concept. It is estimated that HPV is responsible for >95% of cervical carcinoma and ˜70% of oropharyngeal carcinoma⁵². While infection with a high-risk HPV strain (HPV16 & HPV18) is generally necessary for the development of cervical cancer, it is not sufficient and the majority of infections resolve without adverse consequences⁴¹. The use of next-generation sequencing has highlighted the central role HPV integration plays in driving the development of cervical cancer¹⁶. Our results show that PCIP-seq can be applied to identify HPV integration sites in early precancerous samples. This opens up the possibility of generating a more detailed map of HPV integrations as well as potentially providing a biomarker to identify HPV integrations on the road to cervical cancer.

Other potential applications include determining the insertion sites and integrity of retroviral vectors⁵⁴ and detecting transgenes in genetically modified organisms. We envision that in addition to the potential applications outlined above many other novel targets/questions could be addressed using this method.

### REFERENCES

1. Bushman, F. et al. Genome-wide analysis of retroviral DNA integration. Nat Rev Micro 3, 848-858 (2005).
2. Gillet, N. A. et al. The host genomic environment of the provirus determines the abundance of HTLV-1-infected T-cell clones. Blood 117, 3113-3122 (2011).
3. Maldarelli, F. et al. Specific HIV integration sites are linked to clonal expansion and persistence of infected cells. Science (2014). doi:10.1126/science.1254194
4. Wagner, T. A. et al. HIV latency. Proliferation of cells with HIV integrated into cancer genes contributes to persistent infection. Science 345, 570-573 (2014).
5. Bruner, K. M. et al. A quantitative approach for measuring the reservoir of latent HIV-1 proviruses. Nature 566, 1-19 (2019).
6. Einkauf, K. B. et al. Intact HIV-1 proviruses accumulate at distinct chromosomal positions during prolonged antiretroviral therapy. J. Clin. Invest. 129, 988-998 (2019).
7. Rosewick, N. et al. Cis-perturbation of cancer drivers by the HTLV-1/BLV proviruses is an early determinant of leukemogenesis. Nature Communications 8, 15264 (2017).
8. Malhotra, S. et al. Selection for avian leukosis virus integration sites determines the clonal progression of B-cell lymphomas. PLoS Pathog 13, e1006708-25 (2017).
9. Simonetti, F. R. et al. Clonally expanded CD4+ T cells can produce infectious HIV-1 in vivo. Proceedings of the National Academy of Sciences 113, 1883-1888 (2016).
10. Miyazaki, M. et al. Preferential selection of human T-cell leukemia virus type 1 provirus lacking the 5′ long terminal repeat during oncogenesis. Journal of Virology 81, 5714-5723 (2007).
11. Hiener, B. et al. Identification of Genetically Intact HIV-1 Proviruses in Specific CD4+ T Cells from Effectively Treated Participants. Cell Reports 21, 813-822 (2017).
12. Lander, E. S. et al. Initial sequencing and analysis of the human genome. Nature 409, 860-921 (2001).
13. Rivas-Carrillo, S. D., Pettersson, M. E., Rubin, C.-J. & Jern, P. Whole-genome comparison of endogenous retrovirus segregation across wild and domestic host species populations. PNAS 115, 11012-11017 (2018).
14. Pett, M. & Coleman, N. Integration of high-risk human papillomavirus: a key event in cervical carcinogenesis? The Journal of Pathology 212, 356-367 (2007).
15. Hu, Z. et al. Genome-wide profiling of HPV integration in cervical cancer identifies clustered genomic hot spots and a potential microhomology-mediated integration mechanism. Nat Genet 47, 158-163 (2015).
16. Groves, I. J. & Coleman, N. Human papillomavirus genome integration in squamous carcinogenesis: what have next-generation sequencing studies taught us? The Journal of Pathology 245, 9-18 (2018).
17. Sedlazeck, F. J., Lee, H., Darby, C. A. & Schatz, M. C. Piercing the dark matter: bioinformatics of long-range sequencing and mapping. Nature Reviews Genetics 17, 1-18 (2018).
18. Pradhan, B. et al. Detection of subclonal L1 transductions in colorectal cancer by long-distance inverse-PCR and Nanopore sequencing. Scientific Reports 7, 1-12 (2017).
19. Lober, U. et al. Degradation and remobilization of endogenous retroviruses by recombination during the earliest stages of a germ-line invasion. Proceedings of the National Academy of Sciences 5, 201807598-15 (2018).
20. Artesi, M. et al. Monitoring molecular response in adult T-cell leukemia by high-throughput sequencing analysis of HTLV-1 clonality. Leukemia 31, 2532-2535 (2017).
21. Willems, L. et al. In vivo infection of sheep by bovine leukemia virus mutants. Journal of Virology 67, 4078-4085 (1993).
22. Wilm, A. et al. LoFreq: a sequence-quality aware, ultra-sensitive variant caller for uncovering cell-population heterogeneity from high-throughput sequencing datasets. Nucleic Acids Research 40, 11189-11201 (2012).
23. Van den Broeke, A. et al. In vivo rescue of a silent tax-deficient bovine leukemia virus from a tumor-derived ovine B-cell line by recombination with a retrovirally transduced wild-type tax gene. Journal of Virology 73, 1054-1065 (1999).
24. Merimi, M. et al. Complete suppression of viral gene expression is associated with the onset and progression of lymphoid malignancy: observations in Bovine Leukemia Virus-infected sheep. Retrovirology 4,51 (2007).
25. Armitage, A. E. et al. APOBEC3G-Induced Hypermutation of Human Immunodeficiency Virus Type-1 Is Typically a Discrete ‘All or Nothing’ Phenomenon. PLoS Genet 8, e1002550-12 (2012).
26. Samuel, C. E. Adenosine deaminases acting on RNA (ADARs) are both antiviral and proviral. Virology 411, 180-193 (2011).
27. Cachat, A. et al. ADAR1 enhances HTLV-1 and HTLV-2 replication through inhibition of PKR activity. Retrovirology 11, 7415-15 (2014).
28. Rosewick, N. et al. Deep sequencing reveals abundant noncanonical retroviral microRNAs in B-cell leukemia/lymphoma. Proceedings of the National Academy of Sciences 110, 2306-2311 (2013).
29. Durkin, K. et al. Characterization of novel Bovine Leukemia Virus (BLV) antisense transcripts by deep sequencing reveals constitutive expression in tumors and transcriptional interaction with viral microRNAs. Retrovirology 13, 1-16 (2016).
30. Finzi, D. et al. Latent infection of CD4+ T cells provides a mechanism for lifelong persistence of HIV-1, even in patients on effective combination therapy. Nat Med 5, 512-517 (1999).
31. Anderson, E. M. & Maldarelli, F. The role of integration and clonal expansion in HIV infection: live long and prosper. Retrovirology 15, 1-22 (2018).
32. Kiselinova, M. et al. Integrated and Total HIV-1 DNA Predict Ex Vivo Viral Outgrowth. PLoS Pathog 12, e1005472-17 (2016).
33. Folks, T. M., Justement, J., Kinter, A., Dinarello, C. A. & Fauci, A. S. Cytokine-induced expression of HIV-1 in a chronically infected promonocyte cell line. Science 238, 800-802 (1987).
34. Symons, J. et al. HIV integration sites in latently infected cell lines: evidence of ongoing replication. Retrovirology 14, 1-11 (2017).
35. Emiliani, S. et al. Mutations in the tat Gene Are Responsible for Human Immunodeficiency Virus Type 1 Postintegration Latency in the U1 Cell Line. Journal of Virology 72, 1666-1670 (1998).
37. Hughes, J. F. & Coffin, J. M. Human endogenous retrovirus K solo-LTR formation and insertional polymorphisms: implications for human and viral evolution. Proceedings of the National Academy of Sciences 101, 1668-1672 (2004).
38. Cornelis, G. et al. Captured retroviral envelope syncytin gene associated with the unique placental structure of higher ruminants. Proceedings of the National Academy of Sciences 110, E828-E837 (2013).
39. Menzi, F. et al. A transposable element insertion in APOB causes cholesterol deficiency in Holstein cattle. Animal Genetics 47, 253-257 (2016).
40. Arnaud, F. et al. A Paradigm for Virus-Host Coevolution: Sequential Counter-Adaptations between Endogenous and Exogenous Retroviruses. PLoS Pathog 3, e170-14 (2007).
41. Schiffman, M., Castle, P. E., Jeronimo, J., Rodriguez, A. C. & Wacholder, S. Human papillomavirus and cervical cancer. The Lancet 370, 890-907 (2007).
42. Akagi, K. et al. Genome-wide analysis of HPV integration in human cancers reveals recurrent, focal genomic instability. Genome Research 24, 185-199 (2014).
43. Varela, I. et al. Exome sequencing identifies frequent mutation of the SWI/SNF complex gene PBRM1 in renal carcinoma. Nature 469, 1-5 (2011).
44. Jiao, Y. et al. Exome sequencing identifies frequent inactivating mutations in BAP1, ARID1A and PBRM1 in intrahepatic cholangiocarcinomas. Nat Genet 45, 1470-1473 (2013).
45. Quick, J. et al. Real-time, portable genome sequencing for Ebola surveillance. Nature 530, 228-232 (2016).
46. Bangham, C. R. M. Human T Cell Leukemia Virus Type 1: Persistence and Pathogenesis. Annu. Rev. Immunol. 36, annurev-immunol-042617-053222-29 (2017).
47. Goodwin, S. et al. Oxford Nanopore sequencing, hybrid error correction, and de novo assembly of a eukaryotic genome. Genome Research 25, 1750-1756 (2015).
48. Wick, R. Performance of neural network base calling tools for Oxford Nanopore sequencing. Genome Biol 20, 1-10 (2019).
49. Greig, D. R., Jenkins, C., Gharbia, S. & Dallman, T. J. Comparison of single-nucleotide variants identified by Illumina and Oxford Nanopore technologies in the context of a potential outbreak of Shiga toxin-producing Escherichia coli. GigaScience 8, 822-12 (2019).
50. Gillet, N. et al. Mechanisms of leukemogenesis induced by bovine leukemia virus: prospects for novel anti-retroviral therapies in human. Retrovirology 4, 18 (2007).
51. Ko, N. L., Birlouez, E., Wain-Hobson, S., Mahieux, R. & Vartanian, J. P. Hyperediting of human T-cell leukemia virus type 2 and simian T-cell leukemia virus type 3 by the dsRNA adenosine deaminase ADAR-1. Journal of General Virology 93, 2646-2651 (2012).
52. Schiffman, M. et al. Carcinogenic human papillomavirus infection. Nature reviews Disease primers 2, 16086 (2016).
54. Goodwin, L. O. et al. Large-scale discovery of mouse transgenic integration sites reveals frequent structural variation and insertional mutagenesis. Genome Research 29, gr.233866.117-505 (2019).
55. Li, H. Minimap2: pairwise alignment for nucleotide sequences. Bioinformatics 34, 3094-3100 (2018).
56. Rutsaert, S., De Spiegelaere, W., De Clercq, L. & Vandekerckhove, L. Evaluation of HIV-1 reservoir levels as possible markers for virological failure during boosted darunavir monotherapy. Journal of Antimicrobial Chemotherapy (2019).
57. Trypsteen, W. et al. ddpcRquant: threshold determination for single channel droplet digital PCR experiments. Analytical and bioanalytical chemistry 407, 5827-5834 (2015).
58. Li, H. et al. The Sequence Alignment/Map format and SAMtools. Bioinformatics 25, 2078-2079 (2009).
59. Killick, R., Fearnhead, P. & Eckley, I. A. Optimal detection of changepoints with a linear computational cost. Journal of the American Statistical Association 107, 1590-1598 (2012).
60. Thorvaldsdóttir, H., Robinson, J. T. & Mesirov, J. P. Integrative Genomics Viewer (IGV): high-performance genomics data visualization and exploration. Briefings in Bioinformatics 14, 178-192 (2013).
61. Camacho, C. et al. BLAST+: architecture and applications. BMC Bioinformatics 10, 421 (2009).
62. Quinlan, A. R. & Hall, I. M. BEDTools: a flexible suite of utilities for comparing genomic features. Bioinformatics 26, 841-842 (2010).
63. Loman, N. J., Quick, J. & Simpson, J. T. A complete bacterial genome assembled de novo using only nanopore sequencing data. Nature Methods 12, 733-735 (2015).
64. Tjalma WAA, Kim E, Vandeweyer K. The impact on women's health and the cervical cancer screening budget of primary HPV screening with dual-stain cytology triage in Belgium. Eur J Obstet Gynecol Reprod Biol. 2017; 212: 171-181.
65. Tjalma W, Brasseur C, Top G, Ribesse N, Morales I, Van Damme P A. HPV vaccination coverage in the federal state of Belgium according to regions and their impact. Facts Views Vis Obgyn. 2018; 10: 101-105.
66. Mirabello L, Yeager M, Yu K, Clifford G M, Xiao Y, Bin Zhu, et al. HPV16 E7 Genetic Conservation Is Critical to Carcinogenesis. Cell. Elsevier Inc; 2017; 170: 1164-1172.e6. doi:10.1016/j.ce11.2017.08.001
67. Chen, J., Kadlubar, F. F. & Chen, J. Z. DNA supercoiling suppresses real-time PCR: a new approach to the quantification of mitochondrial DNA damage and repair. Nucleic Acids Res 35, 1377-1388 (2007).

1. A method for detecting an integration pattern of human papillomavirus (HPV) in genomic DNA of a subject, the method comprising:
(a) fragmenting genomic DNA isolated from a sample of the subject;
(b) circularizing the DNA fragments to generate circular DNA;
(c) removing non-circularized DNA fragments;
(d) linearizing the circular DNA using an RNA-guided DNA endonuclease and at least one guide RNA or at least one pool of guide RNAs, which target a region in the viral genome, to generate linearized DNA molecules;
(e) amplifying the linearized DNA molecules by an inverse amplification reaction using a pair of primers arranged about and oriented outwardly with respect to the linearization site;
(f) sequencing the amplified DNA;
(g) mapping the sequenced DNA to human genomic DNA sequence; and
(h) optionally mapping the sequenced DNA to the HPV genome.

2. The method according to claim 1, wherein the genomic DNA is fragmented DNA fragments having an average size of about the HPV genome size.

3. The method according to claim 1, wherein the amplification reaction comprises long range PCR.

4. The method according to claim 1, wherein:
a first portion of the circular DNA is linearized using a first guide RNA or a first pool of guide RNAs that target a first region of the viral DNA to generate a first set of linearized DNA molecules; and
a second portion of the circular DNA is linearized using a second guide RNA or a second pool of guide RNAs that target a second region of the viral DNA to generate a second set of linearized DNA molecules,
wherein the first region and the second region of the viral DNA do not overlap.

5. The method according to claim 1, wherein the linearized DNA molecules are amplified using tailed primers, followed by a second amplification using a set of indexing primers to allow multiplexed sequencing of the amplified DNA.

6. The method according to claim 1, wherein the sample comprises cervical or vaginal epithelial cells, such as wherein the sample is a pap smear, or wherein the sample comprises oropharyngeal epithelial cells, such as wherein the sample is an oropharyngeal swab.

7. The method according to claim 1, wherein the HPV is a high-risk HPV strain, a HPV strain 18 or a HPV strain 16.

8. The method according to claim 1, wherein the at least one guide RNA or the at least one pool of guide RNAs target a region of the viral DNA comprising E6 gene and/or E7 gene.

9. The method according to claim 1, wherein the HPV is a HPV strain 18 and wherein:
the first guide RNA or the first pool of guide RNAs to generate the first set of linearized DNA molecules comprises at least one guide RNA selected from the group consisting of: a guide RNA comprising the targeting domain of SEQ ID NO:232, a guide RNA comprising the targeting domain of SEQ ID NO:233, and a guide RNA comprising the targeting domain of SEQ ID NO:234;
the second guide RNA or the second pool of guide RNAs to generate the second set of linearized DNA molecules comprises at least one guide RNA selected from the group consisting of: a guide RNA comprising the targeting domain of SEQ ID NO:235, a guide RNA comprising the targeting domain of SEQ ID NO:236 and a guide RNA comprising the targeting domain of SEQ ID NO:237,
wherein the T in the targeting domains is replaced by U in the guide RNAs;
the first set of linearized DNA molecules are amplified using a primer pair comprising a primer comprising the sequence set forth in SEQ ID NO: 120 (ctccaacgacgcagagaaacac) and a primer comprising the sequence set forth in SEQ ID NO:121 (ggattcaacggtttctggcacc); and/or
the second set of linearized DNA molecules are amplified using a primer pair comprising a primer comprising the sequence set forth in SEQ ID NO: 122 (ttttggttcaggctggattgcg) and a primer comprising the sequence set forth in SEQ ID NO:123 (agaatacacacagctgccaggt).

10. The method according to claim 1, wherein the HPV is a HPV strain 16 and wherein:
the first guide RNA or the first pool of guide RNAs to generate the first set of linearized DNA molecules comprises at least one guide RNA selected from the group consisting of: a guide RNA comprising the targeting domain of SEQ ID NO:238, a guide RNA comprising the targeting domain of SEQ ID NO:239, and a guide RNA comprising the targeting domain of SEQ ID NO:240;
the second guide RNA or the second pool of guide RNAs to generate the second set of linearized DNA molecules comprises at least one guide RNA selected from the group consisting of: a guide RNA comprising the targeting domain of SEQ ID NO:241, a guide RNA comprising the targeting domain of SEQ ID NO:242 and a guide RNA comprising the targeting domain of SEQ ID NO:243,
wherein the T in the targeting domains is replaced by U in the guide RNAs;
the first set of linearized DNA molecules are amplified using a primer pair comprising a primer comprising the sequence set forth in SEQ ID NO:124 (AACCGGACAGAGCCCATTACAA) and a primer comprising SEQ ID NO:125 (AGTCATATACCTCACGTCGCAGT); and/or
the second set of linearized DNA molecules are amplified using a primer pair comprising a primer comprising the sequence set forth in SEQ ID NO: 126 (ACTGGCTTTGGTGCTATGGACT) and a primer comprising SEQ ID NO:127 (CAAACCAGCCGCTGTGTATCTG).

11. A kit for detecting an integration pattern of human papillomavirus (HPV) in genomic DNA of a subject according to claim 1, the kit comprising:
at least one first guide RNA or at least one first pool of guide RNAs, which target a first region in the viral genome; and/or,
a pair of primers arranged about and oriented outwardly with respect to a first linearization site in the viral genome defined by the at least one first guide RNA or at least first one pool of guide RNAs.

12. The kit for detecting an integration pattern of human papillomavirus (HPV) in genomic DNA of a subject of claim 11, the kit further comprising:
at least one second guide RNA or at least one second pool of guide RNAs, which target a second region of the viral DNA, wherein the second region of the viral DNA does not overlap with the first region; and/or,
a pair of primers arranged about and oriented outwardly with respect to a second linearization site in the viral genome defined by the at least one second guide RNA or at least one second pool of guide RNAs.

13. The kit of claim 11 further comprising a DNA polymerase for long range PCR.

14. The kit of claim 11 further comprising an RNA-guided DNA endonuclease.

15. The kit of claim 11 for detecting an integration pattern of a HPV strain 18 wherein:
the first guide RNA or the first pool of guide RNAs comprise at least one guide RNA selected from the group consisting of: a guide RNA comprising the targeting domain of SEQ ID NO:232, a guide RNA comprising the targeting domain of SEQ ID NO:233, and a guide RNA comprising the targeting domain of SEQ ID NO:234;
the second guide RNA or the second pool of guide RNAs comprises at least one guide RNA selected from the group consisting of: a guide RNA comprising the targeting domain of SEQ ID NO:235, a guide RNA comprising the targeting domain of SEQ ID NO:236 and a guide RNA comprising the targeting domain of SEQ ID NO:237,
wherein the T in the targeting domains is replaced by U in the guide RNAs;
a primer pair comprising a primer comprising the sequence set forth in SEQ ID NO:120 and a primer comprising SEQ ID NO:121; and/or
a primer pair comprising a primer comprising the sequence set forth in SEQ ID NO: 122 and a primer comprising SEQ ID NO:123.

16. The kit of claim 11 for detecting an integration pattern of a HPV strain 16 comprising:
the first guide RNA or the first pool of guide RNAs comprises at least one guide RNA selected from the group consisting of: a guide RNA comprising the targeting domain of SEQ ID NO:238, a guide RNA comprising the targeting domain of SEQ ID NO:239, and a guide RNA comprising the targeting domain of SEQ ID NO:240;
the second guide RNA or the second pool of guide RNAs comprises at least one guide RNA selected from the group consisting of: a guide RNA comprising the targeting domain of SEQ ID NO:241, a guide RNA comprising the targeting domain of SEQ ID NO:242 and a guide RNA comprising the targeting domain of SEQ ID NO:243,
wherein the T in the targeting domains is replaced by U in the guide RNAs;
a primer pair comprising a primer comprising the sequence set forth in SEQ ID NO:124 and a primer comprising SEQ ID NO:125; and/or
a primer pair comprising a primer comprising the sequence set forth in SEQ ID NO: 126 and a primer comprising SEQ ID NO:127.

17. A method for monitoring the progression of a human papillomavirus (HPV) infection in a subject comprising:
detecting an integration pattern of human papillomavirus (HPV) in genomic DNA isolated from a sample of the subject according to the method of claim 1; and
comparing the integration pattern with an integration pattern of HPV in genomic DNA isolated from a sample of the subject at an earlier point in time.

18. A method for assessing a risk of having or developing a cancer in a subject comprising:
detecting an integration pattern of human papillomavirus (HPV) in genomic DNA of the subject according to the method of claim 1; and
determining whether the integration pattern predisposes the subject to cancer or cancer development.

19. The method according to claim 18, wherein the cancer is cervical carcinoma or an oropharyngeal carcinoma.

20. The method according to claim 18, further comprising a step of determining whether the integration pattern is indicative of clonal expansion.


SEQUENCE LISTING (247 sequences)

SEQ ID NO: 1 - DNA, 8721 bp, Artificial Sequence
  pBLV344 provirus
tgtatgaaagatcatgccggcctaggcgccgccaccgccccgtaaaccagacagagacgtcagctgccagaaaagctggtgacggcagctggtggctagaatccccgtacctccccaacttcccctttcccgaaaaatccacaccccgagctgctgacctcacctgctgataaaacaataaaatgccggccctgtcgagttagcggcaccagaagcgttctcctcctgagaccctcgtgctcagctctcggtcctgagctctcttgctcccgagaccttctggtcggctatccggcagcggtcaggtaaggcaaaccacggtttggagggtggttctcggctgagaccaccgcgagctctatctccggtcctctgaccgtctccacgtggactctctctcttgcctcctgaccccgcgctccaagggcgtctggcttgcacccgcgcttgtttcctgtcttactttctgtttctcgcggcccgcgctctctccttcggcgccctctagcggccaggagagaccggcaaacaattgggggctcgtccgggattgatcaccccggaaccctaacaatcctctggacccaccccctcggcggcgttttgggtctttcctttaaattatatcatgggaaattccccctcctataacccccccgctggtatctccccctcagactggctcaaccttttgcaaagcgcgcaaaggctcaatccgcgaccctctcctagcgattttaccgatttaaaaaattacatccattggtttcataagacccagaaaaaaccatggactttcacttctggtggccccgcctcatgcccacccgggaaattcggccgggttccccttgtcttggccaccctaaacgaagtgctctcaaacgatgagggcgccccgggtgcatcggccccagaagaacaaccccccccttatgacccccccgccgttttgccaatcatatctgaagggaatcgcaaccgccatcgcgcttgggcactccgagaattacaagatattaaaaaagaaattgaaaataaggcaccgggttcgcaagtatggatacaaacactacgacttgcaatcttgcaggccgaccctactcctgctgacctagaacaactttgccaatatattgcttccccggtcgatcaaacggcccacatgaccagcctaacggcagcaatagcagccgctgaagcggccaacacccttcagggttttaatccccaaaacgggaccctgacccaacaatcagctcagcccaacgccggggatcttagaagtcaatatcaaaacctttggcttcaggcctggaaaaatctccctactcgtccttcagtacaaccctggtccaccatcgtccaaggccccgccgagagctatgtagagtttgtcaaccggttacaaatttcattagctgacaaccttcccgacggagtccctaaagaacccattattgactcccttagctatgctaatgctaacaaagaatgccaacaaattttgcaggggcggggcctagtggccgccccggtgggacaaaaactgcaggcttgtgcacattgggcccccaagattaaacagcctgcaatcctcgtccacaccccagggcccaagatgcccgggcctcggcaaccggcccccaaaaggccccccccgggaccatgctatcgatgcctcaaagaaggccattgggcccgggactgtcccaccaagaccaccggcccccctccgggaccttgtcccatatgcaaagatccttcccattggaaacgagactgtccaaccctcaaatcaaaaaactaatagaggggggacttagcgccccccaaaccgtaacccctataacagatcctcttagtgaggctgaattggaatgcttactttctattcctctggctcgcagccgtccctccgtggctgtatacctgtctggcccctggctgcagccctctcagaatcaagcccttatgctcgtggacaccggggctgaaaatacggtcctcccacaaaattggctggttcgagattacccacggatccccgccgcagtgctcggagcggggggagtctcccggaacagatacaattggctacaaggccctctgaccctggctctaaaaccagagggtccctttatcaccatcccaaaaattttagttgacactttcgataaatggcaaattttaggacgggacgtcctctcccgcctacaggcctctatctccatacctgaggaggtacgcccccccatggtaggcgtcctagatgcccccccgagccacattggattagaacatttgcccgccccacctgaggtacctcaattccctttaaactagaacgcctccaggcccttcaagacctggtccatcgctctctggaggcaggttatatctccccctgggacgggccaggcaataatccagtattcccggtacggaaaccaaatggcacctggaggtttgtgcatgatctacgagctacaaatgctcttacaaagcccatcccggcgctctcccccggaccgccagaccttaccgctatccctacacaccttccacatatcatttgcctagatctcaaagatgccttcttccagattccagtcgaagaccgcttccgctcctattttgcttttaccctccctacccccgggggactccaacctcatagacgctttgcctggcgggtcctacctcaaggcttcattaatagcccggctcttttcgaacgggcactacaggaaccccttcgccaagtttccgccgccttctcccagtctcttctggtgtcctatatggacgatatccttatcgcttcgcctacagaagaacaacggtcacaatgttatcaagccctggctgcccgcctccgggacctagggtttcaggtggcgtctgaaaagactcgccagacgccttcgcccgtccccttcctgggacaaatggtccatgaccagattgtcacctatcagtccctacctaccttgcagatctcatccccaatttctcttcaccaattacaggcggtcttgggagacctccagtgggtctccaggggcacacctactacccgccgacccctgcaacttctctactcttcccttaaaggcatcgatgaccctagggccaccatccagctttccccggaacagctacaaggcattgcagagcttcgacaagccctgtcccataacgcaagatctagatataacgagcaagaacccctgctggcctacatacacctaacccgggcggggtccaccctggtactcttccaaaagggcgctcaatttcccctggcctactttcagacccccttgactgacaaccaagcctcaccttggggcctccttctcctgctgggatgccaatacctgcagactcaggccttaagctcttatgccaagcccatactcaaatactatcacaatcttcctaaaacctctctcgacaattggattcaatcatctgaggaccctcgagttcaggagttgttgcgattgtggccccagatttcctctcagggaatacagcccccgggcccctggaagaccttgatcaccagggcagaggtttttttgacgccccagttctctcctgaaccgattcctgcggccctttgcctctttagtgacggggctacaggacgaggagcatattgcctgtggaaagaccaccttttggactttcaggccgttccggctccagagtccgcccaaaagggagaactagcaggtctcttggcgggcttagcagccgccccgcctgaacctttaaatatatgggtagattccaaatacctatactccttgctcagaaccctagttctgggagcttggcttcaacctgaccccgtaccctcctatgccctcctatacaaaagcctcctccgacatccagcaatctttgttggtcatgtccggagccactcctcagcatcccaccctattgcttccctgaacaattatgtagatcaactgctccccttagaaactccagagcaatggcataagctcacccactgcaactctcgggccttgtctcgatggccgaacccacgtatttcggcctgggatccccgttcccccgctacgctatgtgaaacctgtcaaaagctcaatccaactggaggtggaaagatgcgaactattcagagagggtgggccccgaatcatatttggcaggccgatataacccattataaatacaaacagttcacctacgctttgcacgtgtttgtagatacttactctggagctactcatgcctcagcaaagcgagggctcaccactcaaatgaccattgagggccttctggaggccatagtacatctgggtcgtccaaaaaagctaaacactgaccaaggcgcaaactacacctccaaaacctttgtcaggttttgccagcagttcggagtttccctttctcatcacgttccctacaaccccacaagttcagggttggtagaacggacaaatggactgctcaaacttcttttgtctaaatatcacctagacgaaccccaccttcccatgactcaggccctttctcgagccctctggactcacaatcagattaacctcctgccaattctaaagaccagatgggagttacaccattcacccctacttgctgtcatttcagagggcggagaaacacccaagggctctgataaactctttttgtacaagctccccgggcaaaacaatcgtcggtggctaggaccactcccggccctagtcgaagcctcgggaggcgccctcctggctactaacccccccgtgtgggttccctggcgtttgctaaaagccttcaaatgcccaaagaacgacggtcccgaagacgcccacaaccgatcatcagatgggtaagtctcactcttactctcctcgctctctgtcagcccatccagacttggagatgctccctgtccctaggaaatcaacaatggatgacaacatataaccaagaggcaaaattttccatcgccattgaccaaatactagaggctcataatcaatcgcctttctgtcccaggtctcccagatacaccttggactttgtaaatggttatcctaagatctattggccccccccacaagggcgacgccggtttggagccagggccatggtcacatatgattgcgagccccgatgcccttatgtgggggcagatcacttcgactgcccccactgggacaatgcttcccaggccgatcaagggtccttttatgtcaatcatcagattttattcctgcatctcaaacaatgtcatggaattttcactctaacctgggaaatatggggatatgatcccctgatcaccttttctttacataaaatccctgatccccctcaacccgacttccctcagctgaacagtgactgggttccctctgtcaggtcatgggccctgcttttaaatcaaacggcacgggccttcccagactgtgctatatgttgggaaccttcccctccctgggctcccgaaatattagtatataacaaaaccatctccaactctggacccggtctcgccctcccggacgcccaaatcttctgggtcaacacgtccttgtttaacaccacccaaggatggcaccacccttcccagaggttgttgttcaacgtttctcaaggcaacgccttattattaccccctatctccctggttaatctctctacggcttcctccgcccctcctacccgggtcagacgcagtcctgccgcagccctgaccttgggcctagccctgtcagtggggctcactggaattaatgtagccgtgtccgcccttagccatcagagactcacctccctgatccacgttctggagcaagatcagcaacgcttgatcacagcaattaaccagacccactataatttgcttaatgtggcctctgtggtcgcccagaaccgacgggggctagattggttgtacatccggctgggttttcaaagcctatgtcccacgatcaatgaaccttgctgtttcctgcgcattcaaaatgactccattatccgcctcggtgatctccagcctctctcgcaaagagtctctacagactggcaatggccctggaattgggatctggggctcaccgcctgggtgcgagaaaccattcattctgttctaagcctattcctattagccctttttttgctcttcttggccccctgcctgataaaatgcttgacctctcgccttttaaaactcctccggcaggctccccacttccctgaaatctccttcccccctaaacccgattctgattatcaggccttgctaccatccgcgccagagatctactctcacctctcccccaccaaacccgattacatcaaccttcgaccctgcccttgacacccccatgtttcacgcaccctcaggctgtggtggggcactggcttagtggaatagtcagtgtaccatcacaagcctcttcttgctgccagcgccgagttcgaacacagccctaccctgagcctctctgagtgcatgactgagtgtagcgcagagagattgtcgcttctgcgtgtcactcagtcattttttatagccgattggggttcgcgccctcccgttgcctgtgacacggttaagacctctctcacttctgcttcaccatccccctgccagcgttggtctagtggaaagaactaacgctgacgggggcgatttcttgcagctgtgctaagcgagaggctctggtgctggggataagatgcggcccctagcaccacagtctctgcgccttttgggttcgaatcttccccatgcagcttccgctttttacgccctgttgcacaccctttctagagatacctgaaaatctcagctcgcaccccaaggaaggttgtggctcagaggttaaaatagctcggaccgcaacctccctttctttttattccaccctcgcaaggccccgggttctagaccccctaacggaggttcaaaatttcctctactagggggtgctcaggtccaagtgtgcacaacatctcttccaaaaggtcctgatgaacatcttcccatgtaacaagccccagcagagacattccagccacatccagcagcatttgggccgccttctctaacagtgcccataaagtcccttctgtttccacaacggctgcctctgcatcttctatttccacctcggcaccgactcccccgccgagcccttcaagctcttcgggatccattacctgataacgacaaaattatttcttgtcttttaagcaagtgttgttggttgggggccccactctctacatgcctgcccggccctggttttgtccaatgatgtcaccatcgatgcctggtgccccctctgcgggccccatgaacgactccaattcgaaaggatcgacaccacgctcacctgcgagacccaccgtatcacctggaccgccgatggacgacctttcggcctcaatggaacgttgttccctcgactgcatgtctccgagacccgcccccaagggccccgacgactctggatcaactgcccccttccggccgttcgcgctcagcccggcccggtttcactttcccccttcgagcagtcccccttccagccctaccaatgccaattgccctcggcctctagcgatggttgccccatcatcgggcacggccttcttccctggaacagcttagtaacgcatcctgtcctcggaaaagtccttacattaaatcaaatggccaatttttccttactcccccccttcgataccctccttgtggaccccctccggctgtccgtctttgccccggacactaggggagccatacgttatctctccacccttttgacgctatgcccagctacttgtattctacccctaggcgagcccttctctcctaatgtccccatatgccgctttccccgggacaccaatgaacctcccctttcagaattcgagctgccccttatccaaacgcccggcctgtcttggtctgtccccgcgatcgacctattcctaaccggtcccccttccccatgcgaccggttacacgtgtggtccagtcctcaggccttacaacgcttcctccatgaccccacgctcacctggtcagaattggttgctagcgggaaactaagacttgattcacccttaaaattacagctgttagaaaatgaatggctctcccgccttttttgagggggagtcatttgtatgaaagatcatgccggcctaggcgccgccaccgccccgtaaaccagacagagacgtcagctgccagaaaagctggtgacggcagctggtggctagaatccccgtacctccccaacttcccctttcccgaaaaatccacaccccgagctgctgacctcacctgctgataaaacaataaaatgccggccctgtcgagttagcggcaccagaagcgttctcctcctgagaccctcgtgctcagctctcggtcctgagctctcttgctcccgagaccttctggtcggctatccggcagcggtcaggtaaggcaaaccacggtttggagggtggttctcggctgagaccaccgcgagctctatctccggtcctctgaccgtctccacgtggactctctctcttgcctcctgaccccgcgctccaagggcgtctggcttgcacccgcgcttgtttcctgtcttactttctgtttctcgcggcccgcgctctctccttcggcgccctctagcggccaggagagaccggcaaaca

SEQ ID NO: 2 - DNA, 8720 bp, Artificial Sequence
  Bos-1053_BLV_consensus
tgtatgaaagatcatgccgacctaggcgccgccaccgccccgtaaaccagacagagacgtcagctgccagaaaagctggtgacggcagctggtggctagaatccccgtacctccccaacttcccctttcccgaaaaatccacaccctgagctgctgacctcacctgctgataaactaataaaatgccggccctgtcgagttagcggcaccagaagcgttcttctcctgagaccctcgtgctcagctctcggtcctgagctctcttgctcccgagaccttctggtcggctatccggcagcggtcaggtaaggcaaaccacggtttggagggtggttctcggctgagaccaccgcgagctctatctccggtcctctgaccgtctccacgtggactctctcctttgcctcctgaccccgcgctccaagggcgtctggcttgcacccgcgtttgtttcctgtcttactttctgtttctcgcggcccgcgctctctccttcggcgccctctagcggccaggagagaccggcaaacaattgggggctcgtccgggattgatcaccccggaaccctaacaactctctggacccaccccctcggcggcattttgggtctctccttcaaattatatcatgggaaattccccctcctataacccccccgctggtatctccccctcagactggctcaaccttctgcaaagcgcgcaaaggctcaatccgcgaccctctcctagcgattttaccgatttaaagaattacatccattggtttcataagacccagaaaaaaccatggactttcacttctggtggccccacctcatgtccacccgggagattcggccgggttccccttgtcttggccaccctaaacgaagtgctctcaaacgaagggggcgccccgggtgcatcggccccagaagaacaaccccccccttatgacccccccgccgttttgccaatcatatctgaagggaatcgcaaccgccatcgtgcttgggcactccgagaattacaagatatcaaaaaggaaattgaaaataaggcaccgggttcgcaagtatggatacaaacactacgacttgcaatcctgcaggccgaccctactccggctgacttagaacaactttgccaatatattgcttccccggtcgaccaaacggcccatatgaccagcctaacggcagcaattgccgccgctgaagcggccaacaccctccagggttttaacccccaaaacgggaccctaacccaacaatcagctcagcccaacgccggggatcttagaagtcaatatcaaaacctctggcttcaggcctggaaaaatctccctactcgtccttcagtacaaccttggtccaccatcgtccaaggccccgccgaaagctatgtagagtttgtcaaccggttacaaatttcattagctgacaaccttcccgacggagtccctaaggaacccattattgactcccttagttatgcaaatgctaacaaagagtgtcagcaaattttgcaagggcgaggcctagtggccgccccggtggggcaaaagctgcaggcttgcgcacattgggcccccaagatgaaacagcctgcaattctcgtccacaccccagggcccaagatgcccgggcctcggcaaccggcccccaaaaggcctcccccaggaccatgctatcgatgcctcaaagaaggccattgggcccgggattgtcctaccaaggccaccggcccccctccgggaccttgccccatatgtaaagatccttcccattggaaacgagactgtccaaccctcaaatcaaaaaactaatagaggggggacttagcgccccccaaaccataacacccataacagattctcttagtgaggccgaattagaatgcttactttctattcctctggctcgcagccgtccctccgtggctgtatacctgtctggcccctggctgcagccctctcagaatcaagccctcatgcttgtggacaccggggctgaaaatacggttctcccacaaaattggctggttcgggattacccacggatccccgccgcagtgctcggagcagggggagtctcccggaacagatacaattggctacaaggccctctgaccctggctctaaaaccagagggtccctttatcaccatcccaaaaattttagttgacactttcgacaaatggcaaattttaggacgggacgtcctctcccgcctacaggcctctatctccatacctgaggaagtacgcccccctgtggtaggcgtcttggatgcccccccgagccacattggattagaacatctgccccctccacctgaggtgcctcaattccctttaaactagaacgcctccaggcccttcaagacctggtccatcgctctctggaggcaggttatatctccccctgggacgggccaggcaataatccagtcttcccggtacggaaaccaaatggcgcctggaggtttgtgcatgacctacgagctacaaatgctcttacaaagcccattccggcactctcccccggaccgccagaccttaccgctatccctacacaccttccacatatcatttgcctagatctcaaagacgccttcttccagattccagtcgaagaccgcttccgctcctactttgcttttaccctccctacccccgggggactccaacctcatagacgctttgcctggcgggtcctacctcaaggcttcattaacagcccagctcttttcgaacgagcactacaggaacctcttcgccaagtttccgccgccttttcccagtctcttctggtgtcctatatggacgatatccttatcgcttcgcctacagaagaacagcggtcacaatgttatcaagccctggctgcccgcctccgggacctagggtttcaggtggcatccgaaaagactcgccagacgccttcgcccgtccccttcttgggacaaatggtccatgagcagattgtcacctaccagtccctacctaccttgcagatctcatccccaatttctcttcaccaattacaggcggtcttaggagacctccaatgggtctctaggggcacacccactacccgccggcccctgcaacttctctactcttcccttaaaggcatcgatgaccctagggccatcatccagctttccccggaacagctgcaaggcattgcagagcttcgacaagccctgtcccataacgcaagatctagatataacgagcaagaacccctgctagcctacgtacacctaacccgggcggggtccaccctggtactcttccaaaagggcgctcaatttcccctggcctacttccagacccccttgactgacaaccaagcctcaccttggggcctccttctcctgctgggatgccaatacctgcagactcaggccttaagctcgtatgccaagcccatacttaaatattatcacaatcttcctaaaacttctctagacaattggattcaatcatctgaggaccctcgagtccaggagttgctgcaattgtggccccagatttcctctcagggaatacagcccccgggcccttggaagaccttaatcaccagggcagaggtttttttgacgccccagttctcccctgatccgattcctgcggccctttgcctctttagtgacggggctaaaggacgaggagcatattgcttgtggaaagaccaccttttagactttcaggccgttccggctccagaatccgctcaaaagggagaactagcaggtctcttggcgggcttggcagccgccccgcctgaacctgtaaatatatgggtagattccaaatacctgtactctttgctcagaaccctagttctgggagcttggcttcaacctgaccccgtaccctcctacgccctcctatataaaagcctcctccgacatccagcaatctttgttggccatgtccggagccactcttcagcatcccaccctattgcttccctgaacaattatgtagatcaactgcttcccttagaaactccagagcaatggcataagctcacccactgcaactctcgggccttgtctcgatggccgaacccacgtatctctgcctgggacccccgttcccccgctacgctgtgtgaaacctgtcaaaagcttaatccaactggaggaggaaagatgcgaactattcagagagggtgggccccgaaccatatttggcaggccgatataacccattataaatacaaacagttcacctacgctctgcatgtgtttgtagatacttactctggagctactcatgcctcggcgaagcgtgggctcaccactcaaatgaccattgagggccttcttgaggccatagtgcatctgggtcgtccaaaaaggctaaacactgaccaaggtgcaaactacacctccaaaacctttgtcaggttttgccagcagttcggagtttccctttctcatcatgttccctacaaccccacaagttcggggttagtagaacggacaaatggactgctcaaacttcttctatctaaatatcacctagacgaaccccaccttcccatgactcaggccctttctcgagccctctggactcacaatcagattaacctcctaccaattctaaagaccagatgggagctacaccattcacccccacttgctgtcatttcagagggcggagaaacacccaagggctctgataaactctttttgtacaagctccccgggcaaaacaatcgtcggtggctaggaccactcccggccctagtcgaagcctcgggaggcgctctcctggctactgacccccccgtgtgggttccctggcgtttgctgaaagccttcaaatgcctaaagaacgacggtcccgaagacgcccacaaccgatcatcagatgggtaagtctcactctcactctcctcgctctctctcggcccatccagacttggagatgctccctgtccctaggaaaccaacaatggatgacagcatataaccaagaggcagaattttccatctccattgaccaaatactagaggctcataatcagtcacctttctgtgccaagtctcccagatacaccttggactctgtaaatggctatcctaagatctactggccccccccacaagggcggcgccggtttggagccagggccatggtcacatatgattgcgagccccgatgcccttatgtgggggcagatcgcttcgactgcccccactgggacaatgcctcccaggccgatcaaggatccttttatgtcaatcatcagattttattcctgcatctcaaacaatgtcatggaattttcactctaacctgggagatatggggatatgatcccctgatcaccttttctttacataagatccctgatccccctcaacccgactttccccagttgaacagtgactgggttccctctgtcagatcatgggccctgcttttaaatcaaacagcacgggccttcccagactgtgctatatgttgggaaccttcccctccctgggctcccgaaatattagtatataacaaaaccatctccagctctggacccggcctcgccctcccggacgcccaaatcttctgggtcaacgcgtcctcgtttaacaccacccaaggatggcaccacccttcccagaggttgttgttcaatgtttctcaaggcaacgccttgttattacctcctatctccctggttaatctctctacggcttcctccgcccctcctacccgggtcagacgtagtcccgtcgcagccctgaccttaggcctagccctgtcagtggggctcactggaattaatgtggccgtgtctgcccttagccatcagagactcacctccctgatccacgttctggagcaagatcagcaacgcttgatcacagcaattaaccagacccactataatttgcttaatgtggcctctgtggttgcccagaaccgacgggggcttgattggttatacatccggctgggttttcaaagcctatgtcccacaattaatgagccttgctgtttcctgcgcattcaaaatgactccattatccgcctcggtgatctccagcctctctcgcaaagagtctctacagactggcagtggccctggaattgggatctggggctcactgcctgggtgcgagaaaccattcattctgttctaagcctgttcctattagccctttttttgctcttcctggccccctgcctgataaaatgcttgacctctcgccttttaaagctcctccggcaggctccccacttccctgaaatctccttaacccctaaacccgattctgattatcaggccttgctaccatctgcaccagagatctactctcacctctcccccgtcaaacccgattacatcaacctccgaccctgcccttgatacccccgcgtttcacgcacccccaggctgtggtggtgcactggcttagtggaatagtcagtgtaccatcacaagcctcttcttgctgccggcaccgagttcgaacacagccctaccctgaacctctctgagtgcatgactgagtgtagcgcagagagattgtcgcttctgcgtgtcgctcagtcattttttatagccgattggggttcgcgcccttccgttgcctgtgacacagataagacctctctcacttctgcttcaccatccccctgccagcgttggtctagtggaaagaactaacgctgacgggggcgatttcttgcagctgtgctagcgggaggctctggtgctggggataagatgtggcccttagcaccacagtctctgcgccttttgggttcgaatcttccccacgcagcttccgctttttacgccctgttgcacaccctttctagagatacctgaaaatctcagctcgcaccctgaggaaggttgtggctcagaggttaaaatagctcgagccgcaacctccctttctttttattccaccctcgcaaggccccgggttctgagccccctaacggaggttcaaaatttcctctactaggggatgctcgggtccaagtgtgcacaatatctcttccaaaaggtcctgatgaacgtcttcccatgtaacaagccccagcagagacattccagccacatccagcagcatttgggccgccttttctaacagtgcccataaagtcccttccgtttccacaacggctgcctctgcttcttctatttccacctcggcaccgactcccccgccgagcccttcgagctcttcgggatccattacctgataacgacaaaattatttcttgtcttttaagcaagtgttgttggttgggggccccactctctacatgcctgcccggccctggttttgtccaatgatgtcaccatcgatgcctggtgccccctctgcgggccccataagcgactccaattcgaaaggatcgacaccacgctcacctgcgagacccaccgtatcaactggaccgccgatggacgaccttgcggcctcaatggaacgttgttccctcgactacatgtctccgagacccgcccccaagggccccgacgactctggatcaactgcccccttccggccgttcgcgctcagcccggcccggtttcactttcccccttcgagcggtcccccttccagccctaccaatgccaattgccctcggcctctagcgacggttgccccattatcgggcacggccttcttccctggaacaacttagtaacgcatcctgtcctcggaaaagtccttatattaaatcaaatggccaatttttccttactcccctccttcgataccctccttgtggaccccctccggctgtccgtctttgccccagacaccaggggagccatacgttatctctccacccttttgacgctatgcccagctacttgtattctacccctaggcgagcccttctctcctaatgtccccatatgccgctttccccgggactccaatgaaccccccctttcagaattcgagctgccccttatccaaacgcccggcctgtcctggtctgtccccgcgatcgacctatttctaaccggtcccccttccccatgcgaccggttacacgtatggtccagtcctcaggccttacagcgcttccttcatgaccctacgctaacctggtccgaattggttgctagcagaaagataagacttgattcccccttaaaattacaactgctagaaaatgaatggctctcccgccttttttgagggggagtcatttgtatgaaagatcatgccgacctaggcgccgccaccgccccgtaaaccagacagagacgtcagctgccagaaaagctggtgacggcagctggtggctagaatccccgtacctccccaacttcccctttcccgaaaaatccacaccctgagctgctgacctcacctgctgataaactaataaaatgccggccctgtcgagttagcggcaccagaagcgttcttctcctgagaccctcgtgctcagctctcggtcctgagctctcttgctcccgagaccttctggtcggctatccggcagcggtcaggtaaggcaaaccacggtttggagggtggttctcggctgagaccaccgcgagctctatctccggtcctctgaccgtctccacgtggactctctcctttgcctcctgaccccgcgctccaagggcgtctggcttgcacccgcgtttgtttcctgtcttactttctgtttctcgcggcccgcgctctctccttcggcgccctctagcggccaggagagaccggcaaaca

SEQ ID NO: 3 - DNA, 8720 bp, Artificial Sequence
  Bos-1439_BLV_consensus
tgtatgaaagatcatgccgacctaggcgccgccaccgccccgtaaaccagacagagacgtcagctgccagaaaagctggtgacggcagctggtggctagaatccccgtacctccccaacttcccctttcccgaaaaatccacaccctgagctgctgacctcacctgctgataaattaataaaatgccggccctgtcgagctagcggcaccagaagcgttcttctcctgagaccctcgtgctcagctctcggtcctgagctctcttgctcccgagaccttctggtcggctatccggcagcggtcaggtaaggcaaaccacggtttggagggtggttctcggctgagaccaccgcgagctctatctccggtcctctgaccgtctccacgtggactctctcctttgcctcctgaccccgcgctccaagggcgtctggcttgcacccgcgtttgtttcctgtcttactttctgtttctcgcggcccgcgctctctccttcggcgccctctagcggccaggagagaccggcaaacaattgggggctcgtccgggattgatcaccccggaaccctaacaactctctggacccaccccctcggcggcattttgggtctctccttcaaattatatcatgggaaattccccctcctataacccccccgctggtatctccccctcagactggctcaaccttctgcaaagcgcgcaaaggctcaatccgcgaccctctcctagcgattttaccgatttaaagaattacatccattggtttcataagacccagaaaaaaccatggactttcacttctggtggccccacctcatgtccacccgggagattcggccgggttccccttgtcttggccaccctaaacgaagtgctctcaaacgaagggggcgccccgggtgcatcggccccagaagaacaaccccccccttatgacccccccgccgttttgccaatcatatctgaagggaatcgcaaccgccatcgtgcttgggcactccgagaattacaagatatcaaaaaggaaattgaaaataaggcaccgggttcgcaagtatggatacaaacactacgacttgcaatcctgcaggccgaccctactccggctgacctagaacaactttgccaatatattgcttccccggtcgaccaaacggcccatatgaccagcctaacggcagcaatagccgccgctgaagcggccaacaccctccagggttttaacccccaaaacgggaccctaacccaacaatccgctcagcccaacgccggggatcttagaagtcaatatcaaaacctctggcttcaggcctggaaaaatctccctactcgtccttcagtacaaccttggtccaccatcgtccaaggccccgccgaaagctatgtagagtttgtcaaccggttacaaatttcattagctgacaaccttcccgacggagtccctaaggaacccattattgactcccttagttatgcaaatgctaacaaagagtgtcagcaaattttgcaggggcgaggcctagtggccgccccggtggggcaaaaactgcaggcttgcgcacattgggcccccaagatgaaacagcctgcaattctcgtccacaccccagggcccaagatgcccgggcctcggcaaccggcccccaaaaggcctcccccaggaccatgctatcgatgcctcaaagaaggccattgggcccgggattgtcctaccaaggccaccggcccccctccgggaccttgccccatatgtaaagatccttcccattggaaacgagactgtccaaccctcaaatcaaaaaactaatagaggggggacttagcgccccccaaaccataacacctataacagattctcttagtgaggccgaattagaatgcttactttctattcctctggctcgcagccgtccctccgtggctgtatacctgtctggcccctggctgcagccctctcagaatcaagccctcatgcttgtggacaccggggctgaaaatacggttctcccacaaaattggctggttcgagattacccacggatccccgccgcagtgctcggagcagggggagtctcccggaacagatacaattggctacaaggccctctgaccctggctctaaaaccagagggtccctttatcaccatcccaaaaattttagttgacactttcgacaaatggcaaattttaggacgggacgttctctcccgcctacaggcctctatctccatacctgaggaagtacgcccccctgtggtaggcgtcttggatgcccccccgagccacattggattagaacatctgccccccccacctgaggtgcctcaattccctttaaactagaacgcctccaggcccttcaagacctggtccatcgctctctggaggcaggttatatctccccctgggacgggccaggcaataatccagtcttcccggtacggaaaccaaatggcgcctggaggtttgtgcatgacctacgagctacaaatgctcttacaaagcccattccggcactctcccccggaccgccagaccttaccgctatccctacacaccttccacatatcatttgcctagatctcaaagatgccttcttccaggttccagtcgaagaccgcttccgctcctactttgctttcaccctccctacccccgggggactccaacctcatagacgctttgcctggcgggtcctacctcaaggcttcattaacagcccagctcttttcgaacgagcactacaggaacctcttcgccaagtttccgccgccttttcccagtctcttctggtgtcctatatggacgatatccttatcgcttcgcctacagaagaacagcggtcacaatgttatcaagccctggctgcccgcctccgggacctagggtttcaggtggcatccgaaaagactcgccagacgccttcgcccgtccccttcttgggacaaatggtccatgagcagattgtcacctaccagtccctacctaccttgcagatctcatccccaatttctcttcaccaattacaggcggtcttaggagacctccaatgggtctctaggggcacacccactacccgccggcccctgcaacttctctactcttcccttaaaggcatcgatgaccctagggccatcatccagctttccccggaacagctgcaaggcattgcagagcttcgacaagccctgtcccataacgcaagatctagatataacgagcaagaacccctgctagcctacgtacacctaacccgggcggggtccaccctggtactcttccaaaagggcgctcaatttcccctggcctactttcagacccccttgactgacaaccaagcctcaccttggggcctccttctcctgctgggatgccaatacctgcagactcaggccttaagctcgtatgccaagcccatacttaaatattatcacaatcttcctaaaacctctctagacaattggattcaatcatctgaggaccctcgagtccaggagttgctgcaattgtggccccagatttcctctcagggaatacagcccccgggcccttggaagaccttaatcaccagggcagaggtttttttgacgccccagttctcccctgatccgattcctgcggccctttgcctctttagtgacggggctacaggacgaggagcatattgcttgtggaaagaccaccttttagactttcaggccgttccggctccagaatccgctcaaaagggagaactagcaggtctcttggcgggcttagcagccgccccgcctgaacctgtaaatatatgggtagattccaaatacctgtactctttgctcagaaccctagttctgggagcttggcttcaacctgaccccgtaccctcctacgccctcctatataaaagcctcctccgacatccagcaatctttgttggtcatgtccggagccactcttcagcatcccaccctattgcttccctgaacaattatgtagatcaactgcttcccttagaaactccagagcaatggcataagctcacccactgcaactcgcgggccttatctcgatggccgaacccacgtatctctgcctgggacccccgttcccccgctacgctgtgtgaaacctgtcaaaagcttaatccaactggaggaggaaagatacgaactattcagagagggtgggccccgaatcatatttggcaggccgatataacccattataaatacaaacagttcacctacgctctgcatgtgtttgtagatacttactctggagctactcatgcctcggcgaagcgtgggctcaccactcaaatgaccattgagggccttcttgaggccatagtgcatctgggtcgtccaaaaaagctaaacactgaccaaggtgcaaactacacctccaaaacctttgtcaggttttgccagcagttcggagtttccctttctcatcatgttccctacaaccccacaagttcggggttagtagaacggacaaatggactgctcaaacttcttctatctaaatatcacctagacgaaccccaccttcccatgactcaggccctttctcgagccctctggactcacaatcagattaacctcctaccaattctaaagaccagatgggagctacaccattcacccccacttgctgtcatttcagagggcggagaaacacccaagggctctgataaactctttttgtacaagctccccgggcaaaacaatcgtcggtggctaggaccactcccggccctagttgaagcctcgggaggcgctctcctggctactgacccccccgtgtgggttccctggcgtttgctgaaagccttcaaatgcctaaagaacgacggtcccgaagacgcccacaaccgatcgtcagatgggtaagtctcactctcactctcctcgctctctgtcggcccatccagacttggagatgctccctgtccctaggaaaccaacaatggatgacagcatataaccaagaggcaaaattttccatctccattgaccaaatactagaggctcataatcagtcacctttctgtgccaagtctcccagatacaccttggactctgtaaatggctatcctaagatctactggccccccccacaagggcggcgccggtttggagccagggccatggtcacatatgattgcgagccccgatgcccttatgtgggggcagatcgcttcgactgcccccactgggacaatgcctcccaggccgatcaagggtccttttatgtcaatcatcagattttattcctgcatctcaaacaatgtcatggaattttcactctaacctgggagatatggggatatgatcccctgatcaccttttctttacataggatccctgatccccctcaacccgactttccccggttgagcagtgactgggttccctctgtcagatcatgggccctgcttttaaatcaaacagcacgggccttcccagactgtgctatatgttgggaaccttcccctccctgggctcccgaaatattagtatataacaaaaccatctccagctctggacccggcctcgccctcccggacgcccaaatcttctgggtcaacacgtcctcgtttaacaccacccaaggatggcaccacccttcccagaggttgttgttcaatgtttctcaaggcaacgccttgttattacctcctatctccctggttaatctctctacggcttcctccgcccctcctacccgggtcagacgtagtcccgtcgcagccctgaccttaggcctagccctgtcagtggggctcactggaattaatgtggccgtgtctgcccttagccatcagagactcacctccctgatccacgttctggagcaagatcagcaacgcttgatcacagcaattaaccagacccactataatttgcttaatgtggcctctgtggttgcccagaaccgacgggggcttgattggttgtacatccggctgggttttcaaagcctatgtcccacaattaatgagccttgctgtttcctgcgcattcaaaatgactccattatccgccttggtgatctccagcctctctcgcaaagagtctctacagactggcagtggccctggaattgggatctggggctcactgcctgggtgcgagaaaccattcattctgttctaagcctgttcctattagccctttttttgctcttcctggccccctgcctgataaaatgcttgacctctcgccttttaaagctcctccggcaggctccccacttccctgaaatctccttaacccctaaacccgattctgattatcaggccttgctaccatctgcaccagagatctactctcacctctcccccgtcaaacccgattacatcaacctccgaccctgcccttgatacccccgcgtttcacgcacccccaggctgtggtggtgcactggcttagtggaatagtcagtgtaccatcacaagcctcttcttgctgccagcaccgagttcgaacacagctctaccctgagcctctctgagtgcatgactgagtgtagcgcagagagattgtcgcttctgcgtgtcgctcagtcattttttatagccgattggggttcgcgcccttccgttgcctgtgacacagataagacctctctcacttctgcttcaccatccccctgccagcgttggtctagtggaaagaactaacgctgacgggggcgatttcttgcagctgtgctagcgggaggctctggtgctggggataagatgtggcccttagcaccacagtctctgcgccttttgggttcgaatcttccccacgcagcttccgctttttacgccctgttgcacaccctttctagagatacctgaaaatctcagctcgcaccctgaggaaggttgtggctcagaggttaaaatagctcgagccgcaacctccctttctttttattccaccctcgcaaggccccgggttctgagccccctaacggaggttcaaaatttcctctactaggggatgctcgggtccaagtgtgcacaatatctcttccaaaaggtcctgatgaacgtcttcccatgtaacaagccccagcagagacattccagccacatccagcagcatttgggccgccttttctaacagtgcccataaagtcccttccgtttccacaacggctgcctctgcatcttctatttccacctcggcaccgactcccccgccgagcccttcgagctcttcgggatccattacctgataacgacaaaattatttcttgtcttttaagcaagtgttgttggttgggggccccactctctacatgcctgcccggccctggttttgtccaatgatgtcaccatcgatgcctggtgccccctctgcgggccccatgagcgactccaattcgaaaggatcgacaccacgctcacctgcgagacccaccgtatcaactggaccgccgatggacgaccttgcggcctcaatggaacgttgttccctcgactgcatgtctccgagacccgcccccaagggccccgacgactctggatcaactgcccccttccggccgttcgcgctcagcccggcccggtttcactttcccccttcgagcggtcccccttccagccctaccaatgccaattgccctcggcctctagtgacggttgccccattatcgggcacggccttcttccctggaacaacttagtaacgcatcctgtcctcggaaaagtccttatattaaatcaaatggccaatttttccttactcccctccttcgataccctccttgtggaccccctccggctgtccgtctttgccccagacaccaggggagccatacgttatctctccacccttttgacgctatgcccagctacttgtattctacccctaggcgagcccttctctcctaaggtccccatatgccgctttccccgggactccaatgaaccccccctttcagaattcgagctgccccttatccaaacgcccggcctgtcttggtctgtccccgcgatcgacctattcctaaccggtcccccttccccatgcgaccggttacacgtatggtccagtcctcaggccttacagcgcttccttcatgaccctacgctaacctggtccgaattggttgctagcagaaaaataagacttgattcccccttaaaattacaactgctagaaaatgaatggctctcccgccttttttgagggggagtcatttgtatgaaagatcatgccgacctaggcgccgccaccgccccgtaaaccagacagagacgtcagctgccagaaaagctggtgacggcagctggtggctagaatccccgtacctccccaacttcccctttcccgaaaaatccacaccctgagctgctgacctcacctgctgataaattaataaaatgccggccctgtcgagctagcggcaccagaagcgttcttctcctgagaccctcgtgctcagctctcggtcctgagctctcttgctcccgagaccttctggtcggctatccggcagcggtcaggtaaggcaaaccacggtttggagggtggttctcggctgagaccaccgcgagctctatctccggtcctctgaccgtctccacgtggactctctcctttgcctcctgaccccgcgctccaagggcgtctggcttgcacccgcgtttgtttcctgtcttactttctgtttctcgcggcccgcgctctctccttcggcgccctctagcggccaggagagaccggcaaaca

SEQ ID NO: 4 - DNA, 9656 bp, Artificial Sequence
  HIV_U1_CHRX
tggaagggctaatttggtcccaaagaagacaagatatccttgatctgtggatctaccacacacaaggctacttccctgattggcagaactacacaccagggccagggatcagatatccactgacctttggatggtgcttcaagttagtaccagttgagccagggcaggtagaagaggccaatgaaggagagaacaacaccttgttacaccctatgagcctgcatgggatggaggacccggagggagaagtattagtgtggaagtttgacagcctcctagcatttcgtcacatggcccgagagctgcatccggagtactacaaagactgctgacatcgagttttctacaagggactttccgctggggactttccagggaggtgtggcctgggcgggactggggagtggcgagccctcagatgctgcatataagcagctgctttttgcctgtactgggtctctctggttagaccagatctgagcctgggagctctctggctagctagggaacccactgcttaagcctcaataaagcttgccttgagtgctacaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatatagactaaaacatatagtatgggcaagcagggaactagaacgattcgcagttaatcctggccttttagagacatcagaaggctgtagacaaatactgagacagctacaaccatcccttcagacaggatcagaagaacgtagatcattatttaatacagtagcagtcctctattgtgtgcatcaaaggatagatgtaaaagacaccaaggaagccttagataagatagaggaagagcaaaacaaaagtaagaaaaaggcacagcaagcagcagctgacacaggaaacagcagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaacataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagacgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaacgaagggtgcccacactaatgatgtgaaacaattaacagaggcagtgcaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagaaaaagaacccatagtaggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcaggattagaagtaaacatagtaacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctgccacctgtagtagcaaaagaaatagtagccagctgtgataaatgtcagctaaaaggagaagccatgcatggacaagtagactgtagccccggaatatggcagctagattgtacacatttagaaggaaaagttatcttggtagcagttcatgtggccagtggatatatagaagcagaagtaattccagcagagacagggcaagaaacagcatacttcctcttaaaattagcaggaagatggccagtaaaaacagtacatacagacaatggcagcaatttcaccagtactacagttaaagccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagagacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatatgtatatttcaaggaaagctaaggactggttttatagacatcactatgaaagtactaatccaaaaataagttcagaagtacacatcccactaggggatgctaaattagtaataacaacatattggggtctgcatacaggagaaagagactggcatttgggtcagggagtctccatagaatggaggaaaaagagatatagcacacaagtagaccctgacctagcagaccaactaattcatctgcactattttgattgtttttcagaatctgctataagaaataccatattaggacgtttagttagtcctaggtgtgaatatcaagcaggacataacaaggtaggatctctacagtacttggcactagcagcattaataaaaccaaaacagataaggccacctttgcctagtgttaggaaactgacagaggacagatggaacaagccccagaagaccaagggccacagagggagccatacaatgaatggacactagagcttttagaggaacttaagagtgaagctgttagacattttcctaggatatggctccataacttaggacaacatatctatgaaacttatggggatacttgggcaggagtggaagccataataagaattctgcaacaactgctgtttattcatttcagtattggatgtcaacatagcagaataggcattattcgacagaggagagcaaggaatggagccagtagatcctagactagagccctggaagcttccaggaagtcagcctaaaactgcttgtaccaattgctattgtaaaaagtgttgctttcattgccaagtttgtttcacaacaaaagccttaggcatctcctatggcaggaagaagcggagacagcgacgaagagctcctcaagacagtgagactcatcaagtttctctatcaaagcagtgagtagtacatgtaatgctttctttcaatagttgtgtggaccatagtgttcatagaatacaggaaagtattaagacaaagaaaagtagacaggataattgatagaataagagaaagagcagaagacagtggcaatgagagcgaaggggaccaggaagaattattagcacttgtggagatggggcacgatgctccttgggatgttaatgatctgtagtgctgcagaacaattgtgggtcacagtctattatggggtacctgtgtggaaagaagcaaccaccactctattttgtgcctcggatgctaaagcatatgacacagaggtacataatgtttgggccacacatgcctgtgtacccacagaccctaacccacaagaagtagtattgcaaaatgtgacagaaaattttaacatgtggaaaaataacatggtagaacaaatgcatgaggatataatcagtttatgggatcaaagcctaaaaccatgtgtaaaattaaccccactctgtgttaccttaaattgcactgatttgacgaatgctacttatgccaatggtagtagtgaggagagaggagaaataagaaattgctctttcaatgtcaccacaatcataagaaataagatacaaaaagaatatgcacttttttatagacttgatatagaaccaatagataaggataatacaagctatacattgataaattgtaacacctcagtcattacacaggcttgtccaaaggtatcctttgaaccaattcccatacattattgtgccccggctggttttgcgattctaaagtgtaacgataagaaattcaatggaacaggaccatgtacaaatgtcagtacagtacaatgtacacatggaattaggccagtagtgtcaactcatctgctattaaatggcagtctagcagaaggagaggtagtaattaggtctgaaaatttcacgaacaatgttaaaaccataatagtacagctgaatgaatctgtagaaattaattgtacaagacccaacaacaatacaagaaaaggtatagctataggaccggggagaacactctatgcaagagaaaaaataataggagatataagacaagcacattgtaaccttagtagagcaaaatggaatgacactttaaaacagatagttacaaaattaaaagaacaatttaggaataaaacaatagtctttaatcaatcctcaggaggggacccagaaattgtaatgcacagttttaattgtggaggggaatttttctactgtaagacaacacagctgtttaatagtacttggctgtttaatagtacttggaatgatactgaaaggtcagataacaatgaaactatcatactcccatgcagaataaaacaaattataaacaggtggcaggaagtaggaaaagcaatatatgcccctcccatcagtggacaaattagatgctcatcaaatattacagggctgctattaacaagagatggtggtgataaagagaacagtaccaccgagatcttcagacctggaggaggagatatgagggacaattggagaagtgaattatataaatataaagtagtaaaaattgagccattaggagtagcacccaccaaggcaaagagaagagtggtgcagagagaaaaaagagcagtgggagcgctaggagctctgtttcttgggttcttgggagcagcaggaagcactatgggcgcagcgtcaatggcgctgacggtacagaccagacaattaatgtctggtatagtgcaacagcaaaacaatttgctgaaggctattgaggcgcaacagcatctgttgcaactcacagtctggggcatcaagcagctccaggcaagagtcctggctgtggaaagatacctaaaggatcaacagctcctagggatgtggggttgctctggaaaactcatctgcaccactactgtgccttggaatgctagttggagtaataaatctctggataagatttgggataacatgacctggatggagtgggagagagaaattgacaattacacaggcttaatatacaccttaattgaagaatcgcagtaccaacaagaaaagaatgaacaagaattattggaattggataagtgggcaagtttgtggaattggtttgacataacaaagtggctgtggtatataaaaatattcataatgatagtaggaggtttaataggtttaagaatagtttttactgtactttctatagtgaatagagttaggcagggatactcaccattatcgttccagacccgcctcccaacccagaggggacccgacaggcccgaaggaatcgaagaagaaggtggagagagagacagagacagatccggaccattagtgaatggattcttagcactcatctgggtcgatctgcggagcctgtgcctcttcagctaccaccgcttgagagacttactcttgattgtaacgaggattgtggaacttctgggacgcagggggtgggaagtcctcaaatattgttggaatctcctgcagtattggggtcaggaactaaagaatagtgctattagtttgcttaatgccacagctgtagcagtagctgaggggacagatagggttatagaagtagcacaaagaattggtaggggtatcctccatatccctagaagaataagacaaggcttagaaattgctttgctataagatgggtggcaagtggtcaaaacgtagtatggctggatggtctgctgtaagagaaagaatgaaacgagctgaaccagcagcagatggggtgggagcagtatctcgagacttggaaaaacatggagcaatcacaagtagcaatacagcagctactaatgctgattgtgcctggctagaagcacaagaggatgaggaggtgggttttccagtcagacctcaggtacctttaagaccaataactcgcagggcagctatggatcttagccactttttaaaagaagaggggggactggaagggctaatttggtcccaaagaagacaagatatccttgatctgtggatctaccacacacaaggctacttccctgattggcagaactacacaccagggccagggatcagatatccactgacctttggatggtgcttcaagttagtaccagttgagccagggcaggtagaagaggccaatgaaggagagaacaacaccttgttacaccctatgagcctgcatgggatggaggacccggagggagaagtattagtgtggaagtttgacagcctcctagcatttcgtcacatggcccgagagctgcatccggagtactacaaagactgctgacatcgagttttctacaagggactttccgctggggactttccagggaggtgtggcctgggcgggactggggagtggcgagccctcagatgctgcatataagcagctgctttttgcctgtactgggtctctctggttagaccagatctgagcctgggagctctctggctagctagggaacccactgcttaagcctcaataaagcttgccttgagtgctacaagtagtgtgtgcccgtctgttgtgtgactctggtaactagagatccctcagacccttttagtcagtgtggaaaatctctagca

SEQ ID NO: 5 - DNA, 9656 bp, Artificial Sequence
  HIV_U1_CHR2
tggaagggctaatttggtcccaaagaagacaagatatccttgatctgtggatctaccacacacaaggctacttccctgattggcagaactacacaccagggccagggatcagatatccactgacctttggatggtgcttcaagttagtaccagttgagccagggcaggtagaagaggccaatgaaggagagaacaacaccttgttacaccctatgagcctgcatgggatggaggacccggagggagaagtattagtgtggaagtttgacagcctcctagcatttcgtcacatggcccgagagctgcatccggagtactacaaagactgctgacatcgagttttctacaagggactttccgctggggactttccagggaggtgtggcctgggcgggactggggagtggcgagccctcagatgctgcatataagcagctgctttttgcctgtactgggtctctctggttagaccagatctgagcctgggagctctctggctagctagggaacccactgcttaagcctcaataaagcttgccttgagtgctacaagtagtgtgtgcccgtctgttgtgtgactctggtaactagagatccctcagacccttttagtcagtgtggaaaatctctagcagtggcgcccgaacagggacctgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatatagactaaaacatatagtatgggcaagcagggaactagaacgattcgcagttaatcctggccttttagagacatcagaaggctgtagacaaatactgagacagctacaaccatcccttcagacaggatcagaagaacgtagatcattatttaatacagtagcagtcctctattgtgtgcatcaaaggatagatgtaaaagacaccaaggaagccttagataagatagaggaagagcaaaacaaaagtaagaaaaaggcacagcaagcagcagctgacacaggaaacagcagccaggtcagccaaaattaccctatagtgcagaacctccaggggcaaatggtacatcaggccatatcacctagaactttaaatgcatgggtaaaagtagtagaagagaagactttcagcccagaagtaatacccatgttttcagcattatcagaaggagccaccccacaagatttaaataccatgctaaacacagtggggggacatcaagcagccatgcaaatgttaaaggagaccatcaatgaggaagctgcagaatgggatagattgcatccagtgcatgcagggcctattgcaccaggccagatgagagaaccaaggggaagtgacatagcaggaattactagtacccttcaggaacaaataggatggatgacacataatccacctatcccagtaggagaaatctataaaagatggataatcctgggactaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcagctacactagaagaaatgatgacagcatgtcagggagtggggggacccggccataaagcaagagttttggctgaagcaatgagccaagtaacaaatccagctaccataatgatacagaaaggcaattttaggaaccaaagaaagactattaagtgttttaattgtggcaaagaagggcacatagccaaaaattgcagggcccctaggaaaaagggctgttggaaatgtggaaaagaaggacaccaaatgaaagattgtactgagagacaggctaattttttagggaaaatctggccttcccacaagggaaggccagggaattttcttcagagcagaccagagccaacagccccaccagaagagagcttcaggtttggggaagagacaacaactccctctcagaagcaggagacgatagacaaggaactgtatcctttagcttccctcagatcactctttggcagcgacccctcgtcacaataaagataggggggcaactaaaggaagctctattagatacaggagcagatgatacagtattagaagaaatgaatttgccaggaagatggaaaccaaaaatgatagggggaattggaggttttatcaaagtaagacagtatgatcagatacccatagaaatctgcggacataaagctataggtacagtattagtaggacctacacctgtcaacataattggaagaaatctgttgactcagattggctgcactttaaattttcccattagtcctattgaaactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcaggattaaaacagaaaaaatcagtaacagtactggatgtgggtgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttggagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcgtggtggacggagtattggcaagccacctggattcctgaatgggagtttgtcaatacccctcccttagtgaagttatggtaccagttagaaaaagaacccatagtaggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcaggattagaagtaaacatagtaacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctgccacctgtagtagcaaaagaaatagtagccagctgtgataaatgtcagctaaaaggagaagccatgcatggacaagtagactgtagccccggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaagccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatatgtatatttcaaggaaagctaaggactggttttatagacatcactatgaaagtactaatccaaaaataagttcagaagtacacatcccactaggggatgctaaattagtaataacaacatattggggtctgcatacaggagaaagagactggcatttgggtcagggagtctccatagaatggaggaaaaagagatatagcacacaagtagaccctgacctagcagaccaactaattcatctgcactattttgattgtttttcagaatctgctataagaaataccatattaggacgtttagttagtcctaggtgtgaatatcaagcaggacataacaaggtaggatctctacagtacttggcactagcagcattaataaaaccaaaacagataaggccacctttgcctagtgttaggaaactgacagaggacagatggaacaagccccagaagaccaagggccacagagggagccatacaatgaatggacactagagcttttagaggaacttaagagtgaagctgttagacattttcctaggatatggctccataacttaggacaacatatctatgaaacttatggggatacttgggcaggagtggaagccataataagaattctgcaacaactgctgtttattcatttcagaattggatgtcaacatagcagaataggcattattcgacagaggagagcaaggaacggagccagtagatcctagactagagccctggaagcatccaggaagtcagcctaaaactgcttgtaccaattgctattgtaaaaagtgttgctttcattgccaagtttgtttcacaacaaaagccttaggcatctcctatggcaggaagaagcggagacagcgacgaagagctcctcaagacagtgagactcatcaagtttctctatcaaagcagtgagtagtacatgtaatgctttctttcaatagttgtgtggaccatagtgttcatagaatacaggaaagtattaagacaaagaaaagtagacaggataattgatagaataagagaaagagcagaagacagtggcaatgagagcgaaggggaccaggaagaattatcagcacttgtggagatggggcacgatgctccttgggatgttaatgatctgtagtgctgcagaacaattgtgggtcacagtctattatggggtacctgtgtggaaagaagcaaccaccactctattttgtgcctcggatgctaaagcatatgacacagaggtacataatgtttgggccacacatgcctgtgtacccacagaccctaacccacaagaagtagtattgcaaaatgtgacagaaaattttaacatgtggaaaaataacatggtagaacaaatgcatgaggatataatcagtttatgggatcaaagcctaaaaccatgtgtaaaattaaccccactctgtgttaccttaaattgcactgatttgacgaatgctacttatgccaatggtagtagtgaggagagaggagaaataagaaattgctctttcaatgtcaccacaatcataagaaataagatacaaaaagaatatgcacttttttatagacttgatatagaaccaatagataaggataatacaagctatacattgataaattgtaacacctcagtcattacacaggcttgtccaaaggtatcctttgaaccaattcccatacattattgtgccccggctggttttgcgattctaaagtgtaacgataagaaattcaatggaacaggaccatgtacaaatgtcagtacagtacaatgtacacatggaattaggccagtagtgtcaactcatctgctattaaatggcagtctagcagaaggagaggtagtaattaggtctgaaaatttcacgaacaatgttaaaaccataatagtacagctgaatgaatctgtagaaattaattgtacaagacccaacaacaatacaagaaaaggtatagctataggaccggggagaacactctatgcaagagaaaaaataataggagatataagacaagcacattgtaaccttagtagagcaaaatggaatgacactttaaaacagatagttacaaaattaaaagaacaatttaggaataaaacaatagtctttaatcaatcctcaggaggggacccagaaattgtaatgcacagttttaattgtggaggggaatttttctactgtaagacaacacagctgtttaatagtacttggctgtttaatagtacttggaatgatactgaaaggtcagataacaatgaaactatcatactcccatgcagaataaaacaaattataaacaggtggcaggaagtaggaaaagcaatatatgcccctcccatcagtggacaaattagatgctcatcaaatattacagggctgctattaacaagagatggtggtgataaagagaacagtaccaccgagatcttcagacctggaggaggagatatgagggacaattggagaagtgaattatataaatataaagtagtaaaaattgagccattaggagtagcacccaccaaggcaaagagaagagtggtgcagagaaaaaaaagagcagtgggagcgctaggagctctgttccttgggttcttgggagcagcaggaagcactatgggcgcagcgtcaatggcgctgacggtacagaccagacaattaatgtctggtatagtgcaacagcagaacaatttgctgaaggctattgaggcgcaacagcatctgttgcaactcacagtctggggcatcaagcagctccaggcaagagtcctggctgtggaaagatacctaaaggatcaacagctcctagggatttggggttgctctggaaaactcatctgcaccactactgtgccttggaatgctagttggagtaataaatctctggataagatttgggataacatgacctggatggagtgggagagagaaattgacaattacacaggcttaatatacaccttaattgaagaatcgcagatccaacaagaaaagaatgaacaagaattattggaattggataagtgggcaagtttgtggaattggtttgacataacaaagtggctgtggtatataaaaatattcataatgatagtaggaggtttaataggtttaagaatagtttttactgtactgtctatagtgaatagagttaggcagggatactcaccattatcgttccagacccgcctcccagcccagaggggacccgacaggcccgaaggaatcgaagaagaaggtggagagagagacagagacagatccggaccattagtgaatggattcttagcactcatctgggtcgatctgcggagcctgtgcctcttcagctaccaccgcttgagagacttactcttgattgtaacgaggattgtggaacttctgggacgcagggggtgggaagccctcaaatattgttggaatctcctgcagtattggggtcaggaactaaagaatagtgctattagtttgcttaatgccacagctgtagcagtagctgaggggacagatagggttatagaagtagcacaaagaattggtaggggtatcctccatatccctagaagaataagacaaggcttagaaattgctttgctataagatgggtggcaagtggtcaaaacgtagtatggctggatggtctgctgtaagagaaagaatgaaacgagctgaaccagcagcagatggggtgggagcagtatctcgagacttggaaaaacatggagcaatcacaagtagcaatacagcagctactaatgctaattgtgcctggctagaagcacacgaggaggaggaggtgggttttccagtcagacctcaggtacctttaagaccaataactcgcaaggcagctatggatcttagccactttttaaaagaagaggggggactggaagggctaatttggtcccaaagaagacaagatatccttgatctgtggatctaccacacacaaggctacttccctgattggcagaactacacaccagggccagggatcagatatccactgacctttggatggtgcttcaagttagtaccagttgagccagggcaggtagaagaggccaatgaaggagagaacaacaccttgttacaccctatgagcctgcatgggatggaggacccggagggagaagtattagtgtggaagtttgacagcctcctagcatttcgtcacatggcccgagagctgcatccggagtactacaaagactgctgacatcgagttttctacaagggactttccgctggggactttccagggaggtgtggcctgggcgggactggggagtggcgagccctcagatgctgcatataagcagctgctttttgcctgtactgggtctctctggttagaccagatctgagcctgggagctctctggctagctagggaacccactgcttaagcctcaataaagcttgccttgagtgctacaagtagtgtgtgcccgtctgttgtgtgactctggtaactagagatccctcagacccttttagtcagtgtggaaaatctctagca

SEQ ID NO: 6 - DNA, 6777 bp, Artificial Sequence
  BosT_ERV_chr11_APOB 6777 bp
tgcggggagccggtgaggcattccactcgtgacaaaggtcatgaggaaggaggctcggcatacgcaaaggcgggatcgagcctcaggagtccccccggatattctcgagcattttcccccaaaaaaccagagtctgcctactttattgctttgtgctctcacctctgactttactgggggctgtcccctaccaccgtctctctctctctgtgtcaaagagttaacttacagctccaattaataaagttcctgggcaattaggagtgtttaaatccaaacccctctgatggctctctaactcgcctgacaagtttacccggactcctgcagctatgcatacgattgtttacagtctcccagcctcgagaggcatgggaagcttaagatattcaaatagcttagagcctctcagagagttaaaaactgtcagaataaactagtaaaggatttcattgatgagtcaatgcttgttgccaagttttcacatcccctgaattgtatccttgaatatgtatcaattaatagtgggtatgtagaaaaaataagtagtggccttggtgttagtaactttagacccttaaggtaataaattctttctttgttgtaaacccattacacatccgccctataggaatgcaattttatctttggaagatggtgccaaaccttgaaataattactcttagagaaagtaagtctttgttgataagtccttgtcaagagtcataaaatgttagtaggccttctggccagaagatgatgtaaatcacctaaaccatttgtatacgatacatttgcaggaaagaaaccttggtttttgataagaatcaaagactgctgactttgcatcccctattatcctctatgtgtaacttagggtataaaagcccctgttaaaaataaagctacgggccttgctcaccaacgcttggtctccccatgtcattcttttaacttccagctgagtctccatctggagcgcggaacccaccacgcttactaatcatgcctgggcttctaagacccactcgagaaggtgtctagggtgagacaccttccgctattcgagagggcgcctgcggcctacgtaagtggtgcaaacttcttgtcttgaagttttattggtctcccgcgtaaaccaagctactcagcttcttttctccactgaaatttcctactgagctatcctcattctattgttctctatatccctaattagcatataaatagtcgccgacgccgtctccccttcgaataccctggatcagccggggctggtcctcggcaggtggcgcccgatacagggatttcgaaggtaagtccccaaccccattcccccagtgttgagttttcgggacggataggaccccacttagggtgctgcagacccccctgtagaatagacagggagagaggagtgggaagtgttgaaagtgttgaagagttagagagaaaaaacgatttctaaaaaggctgacaaaaaaggcctaatcttttgatagctaaaagcaaaatcttttactatacttaattttctgacatgggtaatactgaatcaaatgaaagacagctctttataggagtaattttacagttattaggtaaaagaggaattaaagttaaaaaatctgccattcaatcatttctttcatttgtacaagagcactgtccctggtttccagacgaaggctctgttaacttagatgtctgggaaaaagtaggaaaacagttaaaaacttaccatgcagaacatggctcagaaaaggtgcctaatgacgccttttccttatggaatattattagagatgtcttagaccctgccccagattcagaaaaagtacatcttaaaagggatagtgaagaaaatgctgtagttaaacctacccctgaacctaaaaaagtaacctttaaagaggaaaatgaagtcgaaatcgtagttaaacctgaagaaaatgagaaaaacgaagacccgcctgactatcggcaactaagaaaaatgttaatagccgtgactactcaggaacaacctaaagatagggatgaggaacaagatcagccttccccaaaacaaaaagagaatttaggcgagataacaactaaatatcactctgatggagattggcatctcttaaacaaagatgccccaaaaggcctaagagaaacttcccctgtcagaccatccgctcctaggagcttgagggaaacatccccgatcagacagtttgtaagatttagtcaccaaacagttaaaggatctccggagcaggagaagagaaataggggacgctcccatccctccatgcctcctcctctgtgtgggggtaaagggcctccactaggagtttccccgagaagggtttgctctagtcccaaggataaatttgatcctcaccttgaggcttcttttctagtcccctctgcagtttagatagggtaaagaaggtacaaaataaataaaaatattctcaaaaaaaaaaaaataaataaaaaaaaaaatgggtttctctgcatctaagaatatacaaatgtttattggctctgtgaaatatgattagaaatgtcctggacccctctcatgaggctgttaaatagcctttgagggaggctatagcggtagatggtggtgagtctccagtttctgcatagatcataatttctgctattggaaaaaaaaaaaaaaaggagggagaaagtgctttacctcccgagcaaggagagggtttacaggacgctgcggccgagcgccctggcgagctccctccccctctgcgcaaacctttaaaaaacttatccaccactttctgatttaaggtgatcgccaccgcctccgtttgtgcctcccaggacccaggagttccccactttgcccccaaagcctctcaaagcgttgcctaagcctgaggaagataaaaagttttttgaacagtggagcttttaaaacagactgcgtgcacaatatgcagagcatgcttcttagagaaaaccccctcagggggggtctcacccaggctaggagaaaagcgaaaccgcaaggggatttagcaacaatattaacccctgatgtgccgcttactccaattccaacgggagtggctggccccctacctttggacattgtaagatctgtcctggggcatagctcgctttcttttcaaaaaaaaaaaaaaaaaaaattcggtggtgcatggtgtagtagattctgattatattgagaaattaaagtcttgatatcaccgcctaccaaaactgtacaaattaataaaggtcaaagagtaacacagcctttgcttttaccttattatcagacaagaaaaaacttgacttctcaagttaagagccacgaaacatttggatctagtgatctagccttttggatgcaggaaattacagctcctggccaacacatactactgaaaatgagttggtgggattagagaaagtggtatgtgaggtggggatgctatacaggtggtacatttaacaatactttatatattgttataaatacataatataaatatatttgcctaattacagtttgcctaattaggtatgggtataaataaaatataataaaggtatacctaattctacttatagatctatacttaatttgtatataaattaatatgtatactatatatacatatatatatactgtataattaaatgtatattgcagtaatataatgtgtgtggctgatattaattggtatggattggtgtgctgtgatgatatatgttctatatactgtataattgtatatgtgtgacatgcattattgcagtacattggtttgtgttggttggtattagctgtgtatgtgttgtattgctgtaatatatattaattaatatattaattataaagattaattcaaatatattgatttatatatattatatgtcaataagtttatacttgttgccatatataaatacatatataaatacattttacatttaggtatgtctgtataccaaaatgagataaggaggttatacatttattacttaaatttatacagagacctaaactaaacattagacctaaattaacatgtccctaggaaaacagctggacaaatagtaaagttatgtccaaattgtaattaatcacttaaaggtaaacacacaggggcagactacaatgatgagacactagatgcctgaattacaggctacgactcggcccctggtcaagtaaaaagacctccttactggagagtgtaaaaggccagatgtgttgctaacttgtgggagagggtatgcttatatatttctacagaatgcagattctccgatttggatctcagaaaaattcgtcatgtcactttccccaaagaccaggttcgcggcgcgcgcactcgctcgctccccctcccccagccagctctctccgccgccagcggcgccctgcaccctccttgctgcaccccgagacctagagcaaagaagtctgtgcggcgagtgagggccagagaggaaagcgcgcccgcgcagtgcagtccagaccagcgcgctcccgccgcgggcgacaggaaaacgccccgagcccaagcggcgcggctagcccgagtccgcgaaccccgcccctccgcccgccgtagacgcctcggcccacaacctgccgttcgcgttctgcctcctgatgctgcggacactgctccgccgagccgatgcctacagctgctccttgtctcatcttgtttaaatcctcaatataatgtttgctcctttgtaatgttacaatgtccaccttatcttatgatgtaaccacttattgaattatgctcttgctgtattacaacaactgcaggatttaatgcgaacacgacggtttgtgggcttacttatcttgggaatagcagctttgataagcgcaattacttctgttactgtggcagcaatatcattgactcaacaagtacatactgctcaatatgttgattctatgtccaaaaatgtttctttagcattggcaacacaggaagctatagacaggaaattagaaatgagggtagatgccctagaggaagcagtaatacatattgggactgaattgcaggctttaaaggtgaaaatggcattgtcctgtcatgctgactaccggtggatatgtgtaacacccctgaaagtaaatgagacagattttgaatgggaaaagattaaaaaccatatttcaggtatttggaacagctctgacattagcttagacttagggaaacttcacaatcaaatagcaaccctggaacactcccgattagattttactgccgctggaacagcaaatgatttcttccatactttctctaactacatttcaggaaaaaatattctgtctaccttcctcggctacgctaccttggctgttttaattttatttctaataatcattcttccttgtattgtcaggattcttcggcagagcattcagaggctcgcgactgagctacatctggctgttttaagaaataaaaaagggggagatgcggggagccggtgaggcattccactcgtgacaaaggtcatgaggaaggaggctcggcatacgcaaaggcgggatcgagcctcaggagtccccccggatattctcgagcattttcccccaaaaaaccagagtctgcctactttattgctttgtgctctcacctctgactttactgggggctgtcccctaccaccgtctctctctctctgtgtcaaagagttaacttacagctccaattaataaagttcctgggcaattaggagtgtttaaatccaaacccctctgatggctctctaactcgcctgacaagtttacccggactcctgcagctatgcatacgattgtttacagtctcccagcctcgagaggcatgggaagcttaagatattcaaatagcttagagcctctcagagagttaaaaactgtcagaataaactagtaaaggatttcattgatgagtcaatgcttgttgccaagttttcacatcccctgaattgtatccttgaatatgtatcaattaatagtgggtatgtagaaaaaataagtagtggccttggtgttagtaactttagacccttaaggtaataaattctttctttgttgtaaacccattacacatccgccctataggaatgcaattttatctttggaagatggtgccaaaccttgaaataattactcttagagaaagtaagtctttgttgataagtccttgtcaagagtcataaaatgttagtaggccttctggccagaagatgatgtaaatcacctaaaccatttgtatacgatacatttgcaggaaagaaaccttggtttttgataagaatcaaagactgctgactttgcatcccctattatcctctatgtgtaacttagggtataaaagcccctgttaaaaataaagctacgggccttgctcaccaacgcttggtctccccatgtcattcttttaacttccagctgagtctccatctggagcgcggaacccaccacgcttactaatcatgcctgggcttctaagacccactcgagaaggtgtctagggtgagacaccttccgctattcgagagggcgcctgcggcctacgtaagtggtgcaaacttcttgtcttgaagttttattggtctcccgcgtaaaccaagctactcagcttcttttctccactgaaatttcctactgagctatcctcattctattgttctctatatccctaattagcatataaatagtcgccgacgccgtctccccttcgaataccctggatcagccggggctggtcctcggca

SEQ ID NO: 7 - DNA, 55 bp, Artificial Sequence
  2563-BLV-Guide31_5PA
ttctaatacgactcactatagtctgagggggagataccagcgttttagagctaga

SEQ ID NO: 8 - DNA, 55 bp, Artificial Sequence
  2564-BLV-Guide32_5PA
ttctaatacgactcactatagaagacccaaaacgccgccgagttttagagctaga

SEQ ID NO: 9 - DNA, 55 bp, Artificial Sequence
  2565-BLV-Guide33_5PA
ttctaatacgactcactatagcaccccctcggcggcgttttgttttagagctaga

SEQ ID NO: 10 - DNA, 55 bp, Artificial Sequence
  2597-BLV-Guide43_3PA
ttctaatacgactcactatagacagccggagggggtccacagttttagagctaga

SEQ ID NO: 11 - DNA, 55 bp, Artificial Sequence
  2598-BLV-Guide44_3PA
ttctaatacgactcactatagttagtaacgcatcctgtcctgttttagagctaga

SEQ ID NO: 12 - DNA, 55 bp, Artificial Sequence
  2599-BLV-Guide45_3PA
ttctaatacgactcactatagccctccttgtggaccccctcgttttagagctaga

SEQ ID NO: 13 - DNA, 55 bp, Artificial Sequence
  2560-BLV-Guide46_3PA
ttctaatacgactcactatagcaaagacggacagccggagggttttagagctaga

SEQ ID NO: 14 - DNA, 55 bp, Artificial Sequence
  2570-BLV-Guide34_5PB
ttctaatacgactcactatagcttctggggccgatgcacccgttttagagctaga

SEQ ID NO: 15 - DNA, 55 bp, Artificial Sequence
  2571-BLV-Guide35_5PB
ttctaatacgactcactatagcgaagtgctctcaaacgatggttttagagctaga

SEQ ID NO: 16 - DNA, 55 bp, Artificial Sequence
  2572-BLV-Guide36_5PB
ttctaatacgactcactatagaacggcgggggggtcataaggttttagagctaga

SEQ ID NO: 17 - DNA, 54 bp, Artificial Sequence
  2584-BLV-Guide40_3PB
ttctaatacgactcactataggttaggaataggtcgatcggttttagagctaga

SEQ ID NO: 18 - DNA, 55 bp, Artificial Sequence
  2585-BLV-Guide41_3PB
ttctaatacgactcactatagtaaccggtcgcatggggaaggttttagagctaga

SEQ ID NO: 19 - DNA, 55 bp, Artificial Sequence
  2586-BLV-Guide42_3PB
ttctaatacgactcactatagaggaagcgttgtaaggcctggttttagagctaga

SEQ ID NO: 20 - DNA, 55 bp, Artificial Sequence; identical to SEQ ID NO: 14
  2570-BLV-Guide34_5PB

SEQ ID NO: 21 - DNA, 55 bp, Artificial Sequence; identical to SEQ ID NO: 15
  2571-BLV-Guide35_5PB

SEQ ID NO: 22 - DNA, 55 bp, Artificial Sequence; identical to SEQ ID NO: 16
  2572-BLV-Guide36_5PB

SEQ ID NO: 23 - DNA, 54 bp, Artificial Sequence; identical to SEQ ID NO: 17
  2584-BLV-Guide40_3PB

SEQ ID NO: 24 - DNA, 55 bp, Artificial Sequence; identical to SEQ ID NO: 18
  2585-BLV-Guide41_3PB

SEQ ID NO: 25 - DNA, 55 bp, Artificial Sequence
  2691-BLV-Guide48_3PB
ttctaatacgactcactatagctgccccttatccaaacgccgttttagagctaga

SEQ ID NO: 26 - DNA, 55 bp, Artificial Sequence
  2652-BosT_ERV_G7-PB5
ttctaatacgactcactatagaggttgttcctgagtagtcagttttagagctaga

SEQ ID NO: 27 - DNA, 55 bp, Artificial Sequence
  2663-BosT_ERV_G8-PB5
ttctaatacgactcactatagtgttcctcatccctatctttgttttagagctaga

SEQ ID NO: 28 - DNA, 55 bp, Artificial Sequence
  2664-BosT_ERV_G9-PB5
ttctaatacgactcactatagacaactaaatatcactctgagttttagagctaga

SEQ ID NO: 29 - DNA, 55 bp, Artificial Sequence
  2657-BosT_ERV_G10-PC3
ttctaatacgactcactatagcaaggtagcgtagccgaggagttttagagctaga

SEQ ID NO: 30 - DNA, 54 bp, Artificial Sequence
  2658-BosT_ERV_G11-PC3
ttctaatacgactcactatagaaatcatttgctgttccaggttttagagctaga

SEQ ID NO: 31 - DNA, 54 bp, Artificial Sequence
  2659-BosT_ERV_G11-PC3
ttctaatacgactcactataggggtgttacacatatccacgttttagagctaga

SEQ ID NO: 32 - DNA, 55 bp, Artificial Sequence
  2627-JSRV_G9-5PA
ttctaatacgactcactatagtcgagaccagccacaacagagttttagagctaga

SEQ ID NO: 33 - DNA, 54 bp, Artificial Sequence
  2628-JSRV_G10-5PA
ttctaatacgactcactatagggttgctttcaaccccctcgttttagagctaga

SEQ ID NO: 34 - DNA, 55 bp, Artificial Sequence
  2629-JSRV_G11-5PA
ttctaatacgactcactatagactattgctttacagaacgcgttttagagctaga

SEQ ID NO: 35 - DNA, 54 bp, Artificial Sequence
  2642-JSRV_G18-3PA
ttctaatacgactcactatagttacagcggatacaaaacggttttagagctaga

SEQ ID NO: 36 - DNA, 55 bp, Artificial Sequence
  2643-JSRV_G19-3PA
ttctaatacgactcactatagaaggctggtacgcgcggcaggttttagagctaga

SEQ ID NO: 37 - DNA, 55 bp, Artificial Sequence
  2644-JSRV_G20-3PA
ttctaatacgactcactatagatgtcgagcacgaattgcatgttttagagctaga

SEQ ID NO: 38 - DNA, 54 bp, Artificial Sequence
  2632-JSRV_G12-5PB
ttctaatacgactcactatagatctttcaaaagtccggcagttttagagctaga

SEQ ID NO: 39 - DNA, 54 bp, Artificial Sequence
  2633-JSRV_G13-5PB
ttctaatacgactcactatagctgatgttaaccgacagcagttttagagctaga

SEQ ID NO: 40 - DNA, 55 bp, Artificial Sequence
  2634-JSRV_G14-5PB
ttctaatacgactcactatagcacaaatatcaaatgcggctgttttagagctaga

SEQ ID NO: 41 - DNA, 54 bp, Artificial Sequence
  2637-JSRV_G15-3PB
ttctaatacgactcactataggctcagacctcttttaggagttttagagctaga

SEQ ID NO: 42 - DNA, 55 bp, Artificial Sequence
  2638-JSRV_G16-3PB
ttctaatacgactcactatagttctgactttccgtgggatagttttagagctaga

SEQ ID NO: 43 - DNA, 55 bp, Artificial Sequence
  2639-JSRV_G17-3PB
ttctaatacgactcactatagattttgtaaaaaattatcgagttttagagctaga

SEQ ID NO: 44 - DNA, 55 bp, Artificial Sequence
  2604-HTLV1_G21-5PA
ttctaatacgactcactatagctggtggaaatcgtaactgggttttagagctaga

SEQ ID NO: 45 - DNA, 55 bp, Artificial Sequence
  2605-HTLV1_G22-5PA
ttctaatacgactcactatagtcccaaaaggataccccggcgttttagagctaga

SEQ ID NO: 46 - DNA, 55 bp, Artificial Sequence
  2606-HTLV1_G23-5PA
ttctaatacgactcactatagtaaaatttcattcacccggcgttttagagctaga

SEQ ID NO: 47 - DNA, 55 bp, Artificial Sequence
  2611-HTLV1_G24-3PA
ttctaatacgactcactatagcggggtggcaaaaaatcacggttttagagctaga

SEQ ID NO: 48 - DNA, 54 bp, Artificial Sequence
  2612-HTLV1_G25-3PA
ttctaatacgactcactatagggtgtacaggttttggggcgttttagagctaga

SEQ ID NO: 49 - DNA, 55 bp, Artificial Sequence
  2613-HTLV1_G26-3PA
ttctaatacgactcactatagtttgccaccccggccagctcgttttagagctaga

SEQ ID NO: 50 - DNA, 55 bp, Artificial Sequence
  2616-HTLV1_G27-5PB
ttctaatacgactcactatagcatgactggaaggacttggggttttagagctaga

SEQ ID NO: 51 - DNA, 54 bp, Artificial Sequence
  2617-HTLV1_G28-5PB
ttctaatacgactcactataggatggtctgcataaactgggttttagagctaga

SEQ ID NO: 52 - DNA, 55 bp, Artificial Sequence
  2618-HTLV1_G29-5PB
ttctaatacgactcactatagcaaactgctgcaccgcaagcgttttagagctaga

SEQ ID NO: 53 - DNA, 54 bp, Artificial Sequence
  2619-HTLV1_G30-3PB
ttctaatacgactcactataggaaatcataggcgtgccatgttttagagctaga

SEQ ID NO: 54 - DNA, 54 bp, Artificial Sequence
  2620-HTLV1_G31-3PB
ttctaatacgactcactataggctggccatctttagggcagttttagagctaga

SEQ ID NO: 55 - DNA, 54 bp, Artificial Sequence
  2621-HTLV1_G32-3PB
ttctaatacgactcactatagaggactgtagtactaaagagttttagagctaga

SEQ ID NO: 56 - DNA, 55 bp, Artificial Sequence
  2622-HTLV1_G33-3PB
ttctaatacgactcactatagatggcacgcctatgatttccgttttagagctaga

SEQ ID NO: 57 - DNA, 55 bp, Artificial Sequence
  2667-HIV_G1-5PA
ttctaatacgactcactatagagagcgtcggtattaagcgggttttagagctaga

SEQ ID NO: 58 - DNA, 54 bp, Artificial Sequence
  2668-HIV_G2-5PA
ttctaatacgactcactatagcgggggagaattagataaagttttagagctaga

SEQ ID NO: 59 - DNA, 55 bp, Artificial Sequence
  2681-HIV_G9-3PA
ttctaatacgactcactatagaggcgggtctggaacgataagttttagagctaga

SEQ ID NO: 60 - DNA, 55 bp, Artificial Sequence
  2682-HIV_G10-3PA
ttctaatacgactcactatagcactcatctgggtcgatctggttttagagctaga

SEQ ID NO: 61 - DNA, 54 bp, Artificial Sequence
  2683-HIV_G11-3PA
ttctaatacgactcactatagaatccattcactaatggtcgttttagagctaga

SEQ ID NO: 62 - DNA, 55 bp, Artificial Sequence
  2671-HIV_G3-5PB
ttctaatacgactcactatagcatgcagggcctattgcaccgttttagagctaga

SEQ ID NO: 63 - DNA, 54 bp, Artificial Sequence
  2672-HIV_G4-5PB
ttctaatacgactcactatagattgcatccagtgcatgcagttttagagctaga

SEQ ID NO: 64 - DNA, 54 bp, Artificial Sequence
  2673-HIV_G5-5PB
ttctaatacgactcactatagcaataggccctgcatgcacgttttagagctaga

SEQ ID NO: 65 - DNA, 55 bp, Artificial Sequence
  2676-HIV_G6-3PB
ttctaatacgactcactatagcaaaacgtagtatgagtggagttttagagctaga

SEQ ID NO: 66 - DNA, 55 bp, Artificial Sequence
  2677-HIV_G7-3PB
ttctaatacgactcactatagctactaatgctaattgtgccgttttagagctaga

SEQ ID NO: 67 - DNA, 55 bp, Artificial Sequence
  2678-HIV_G8-3PB
ttctaatacgactcactatagcgaactgaaccagcagcagagttttagagctaga

SEQ ID NO: 68 - DNA, 54 bp, Artificial Sequence
  1_HPV18_R1_guide1
ttctaatacgactcactatagtgctgcaaccgagcacgacgttttagagctaga

SEQ ID NO: 69 - DNA, 54 bp, Artificial Sequence
  2_HPV18_R1_guide2
ttctaatacgactcactatagtgctcggttgcagcacgaagttttagagctaga

SEQ ID NO: 70 - DNA, 55 bp, Artificial Sequence
  3_HPV18_R1_guide3
ttctaatacgactcactatagcgacgatttcacaacatagcgttttagagctaga

SEQ ID NO: 71 - DNA, 55 bp, Artificial Sequence
  8_HPV18_R2_guide4
ttctaatacgactcactatagattttagaggattggaacttgttttagagctaga

SEQ ID NO: 72 - DNA, 55 bp, Artificial Sequence
  9_HPV18_R2_guide5
ttctaatacgactcactatagtctgctatactgcttaaattgttttagagctaga

SEQ ID NO: 73 - DNA, 54 bp, Artificial Sequence
  10_HPV18_R2_guide6
ttctaatacgactcactatagcatcatattgcccaggtacgttttagagctaga

SEQ ID NO: 74 - DNA, 55 bp, Artificial Sequence
  3261_HPV16_E6-E7_G1
ttctaatacgactcactatagctaattaacaaatcacacaagttttagagctaga

SEQ ID NO: 75 - DNA, 54 bp, Artificial Sequence
  3262_HPV16_E6-E7_G2
ttctaatacgactcactatagattccataatataaggggtgttttagagctaga

SEQ ID NO: 76 - DNA, 54 bp, Artificial Sequence
  3263_HPV16_E6-E7_G3
ttctaatacgactcactatagcaacaagacatacatcgacgttttagagctaga

SEQ ID NO: 77 - DNA, 55 bp, Artificial Sequence
  3266_HPV16_L1_G1
ttctaatacgactcactatagccacctataggggaacactggttttagagctaga

SEQ ID NO: 78 - DNA, 55 bp, Artificial Sequence
  3267_HPV16_L1_G2
ttctaatacgactcactatagacctacctcaacacctacacgttttagagctaga

SEQ ID NO: 79 - DNA, 55 bp, Artificial Sequence
  3268_HPV16_L1_G3
ttctaatacgactcactatagtaatagagaatgtatatctagttttagagctaga

SEQ ID NO: 80 - DNA, 44 bp, Artificial Sequence
  2568-BLV_5PA-minION-F
tttctgttggtgctgatattgcgcgaccctctcctagcgatttt

SEQ ID NO: 81 - DNA, 44 bp, Artificial Sequence
  2595-BLV_5PA-minION-R
acttgcctgtcgctctatcttcgttagggttccggggtgatcaa

SEQ ID NO: 82 - DNA, 44 bp, Artificial Sequence
  2601-BLV_3PA-minION-F
tttctgttggtgctgatattgcctccacccttttgacgctatgc

SEQ ID NO: 83 - DNA, 44 bp, Artificial Sequence
  2602-BLV_3PA-minION-R
acttgcctgtcgctctatcttcattggcattggtagggctggaa

SEQ ID NO: 84 - DNA, 44 bp, Artificial Sequence
  2575-BLV_5PB-minION-F
tttctgttggtgctgatattgccccgccgttttgccaatcatat

SEQ ID NO: 85 - DNA, 44 bp, Artificial Sequence
  2576-BLV_5PB-minION-R
acttgcctgtcgctctatcttctttagggtggccaagacaaggg

SEQ ID NO: 86 - DNA, 44 bp, Artificial Sequence
  2589-BLV_3PB-minION-F
tttctgttggtgctgatattgctcagaattggttgctagcggga

SEQ ID NO: 87 - DNA, 44 bp, Artificial Sequence
  2603-BLV_3PB-minION-R
acttgcctgtcgctctatcttctttggataaggggcagctcgaa

SEQ ID NO: 88 - DNA, 44 bp, Artificial Sequence; identical to SEQ ID NO: 84
  2575-BLV_5PB-minION-F

SEQ ID NO: 89 - DNA, 44 bp, Artificial Sequence; identical to SEQ ID NO: 85
  2576-BLV_5PB-minION-R

SEQ ID NO: 90 - DNA, 43 bp, Artificial Sequence
  2690-BLV_3PB-minION-F
tttctgttggtgctgatattgcggtccagtcctcaggccttac

SEQ ID NO: 91 - DNA, 44 bp, Artificial Sequence; identical to SEQ ID NO: 87
  2603-BLV_3PB-minION-R

SEQ ID NO: 92 - DNA, 44 bp, Artificial Sequence
  2650-BosT_ERV_PB5-F
tttctgttggtgctgatattgcctgtcagaccatccgctcctag

SEQ ID NO: 93 - DNA, 44 bp, Artificial Sequence
  2651-BosT_ERV_PB5-R
acttgcctgtcgctctatcttctagtcaggcgggtcttcgtttt

SEQ ID NO: 94 - DNA, 44 bp, Artificial Sequence
  2655-BosT_ERV_PC3-F
tttctgttggtgctgatattgctcttcggcagagcattcagagg

SEQ ID NO: 95 - DNA, 44 bp, Artificial Sequence
  2656-BosT_ERV_PC3-R
acttgcctgtcgctctatcttcaagtaagcccacaaaccgtcgt

SEQ ID NO: 96 - DNA, 44 bp, Artificial Sequence
  2625-JSRV-5PA-F
tttctgttggtgctgatattgccctccaccgtctgagaacatgt

SEQ ID NO: 97 - DNA, 44 bp, Artificial Sequence
  2626-JSRV-5PA-R
acttgcctgtcgctctatcttcagcatacctgggttccgaatca

SEQ ID NO: 98 - DNA, 44 bp, Artificial Sequence
  2640-JSRV-3PA-F
tttctgttggtgctgatattgcgaaccggacctctcgacattcc

SEQ ID NO: 99 - DNA, 44 bp, Artificial Sequence
  2641-JSRV-3PA-R
acttgcctgtcgctctatcttcaaacacaaacatgccctcgtcc

SEQ ID NO: 100 - DNA, 44 bp, Artificial Sequence
  2630-JSRV-5PB-F
tttctgttggtgctgatattgcgggacctgatgagccttaccag

SEQ ID NO: 101 - DNA, 44 bp, Artificial Sequence
  2631-JSRV-5PB-R
acttgcctgtcgctctatcttcgcaatggtgaatggagcggtag

SEQ ID NO: 102 - DNA, 44 bp, Artificial Sequence
  2635-JSRV-3PB-F
tttctgttggtgctgatattgcccttcattcactgtggcgaagt

SEQ ID NO: 103 - DNA, 44 bp, Artificial Sequence
  2636-JSRV-3PB-R
acttgcctgtcgctctatcttcgtaaggaacacaagctcgggga

SEQ ID NO: 104 - DNA, 44 bp, Artificial Sequence
  2607-HTLV1-5PA-F
tttctgttggtgctgatattgctcatccaaacccaagcccagat

SEQ ID NO: 105 - DNA, 44 bp, Artificial Sequence
  2608-HTLV1-5PA-R
acttgcctgtcgctctatcttcggaccgggttctaggcgatatg

SEQ ID NO: 106 - DNA, 44 bp, Artificial Sequence
  2609-HTLV1-3PA-F
tttctgttggtgctgatattgctctacccgaagactgtttgccc

SEQ ID NO: 107 - DNA, 44 bp, Artificial Sequence
  2610-HTLV1-3PA-R
acttgcctgtcgctctatcttcttgtatgagtgattggcggggt

SEQ ID NO: 108 - DNA, 44 bp, Artificial Sequence
  2614-HTLV1-5PB-F
tttctgttggtgctgatattgcaaagacctccaagacctcctgc

SEQ ID NO: 109 - DNA, 44 bp, Artificial Sequence
  2615-HTLV1-5PB-R
acttgcctgtcgctctatcttccgtaggctcaacatagggaggg

SEQ ID NO: 110 - DNA, 44 bp, Artificial Sequence
  2623-HTLV1-3PB-F
tttctgttggtgctgatattgcctctcacacggcctcatacagt

SEQ ID NO: 111 - DNA, 44 bp, Artificial Sequence
  2624-HTLV1-3PB-R
acttgcctgtcgctctatcttcgagtggtgagggttgagtggaa

SEQ ID NO: 112 - DNA, 44 bp, Artificial Sequence
  2665-HIV-5PA-F
tttctgttggtgctgatattgcaaaattcggttaaggccagggg

SEQ ID NO: 113 - DNA, 44 bp, Artificial Sequence
  2666-HIV-5PA-R
acttgcctgtcgctctatcttcctcgcacccatctctctccttc

SEQ ID NO: 114 - DNA, 44 bp, Artificial Sequence
  2679-HIV-3PA-F
tttctgttggtgctgatattgcgctaccaccgcttgagagactt

SEQ ID NO: 115 - DNA, 44 bp, Artificial Sequence
  2680-HIV-3PA-R
acttgcctgtcgctctatcttcaccaattccacaaacttgccca

SEQ ID NO: 116 - DNA, 44 bp, Artificial Sequence
  2669-HIV-5PB-F
tttctgttggtgctgatattgcccaggccagatgagagaaccaa

SEQ ID NO: 117 - DNA, 44 bp, Artificial Sequence
  2670-HIV-5PB-R
acttgcctgtcgctctatcttctcccattctgcagcttcctcat

SEQ ID NO: 118 - DNA, 44 bp, Artificial Sequence
  2674-HIV-3PB-F
tttctgttggtgctgatattgcgaggaggaggaggtgggttttc

SEQ ID NO: 119 - DNA, 44 bp, Artificial Sequence
  2675-HIV-3PB-R
acttgcctgtcgctctatcttctgaccacttgccacccatctta

SEQ ID NO: 120 - DNA, 22 bp, Artificial Sequence
  4_HPV18_R1_Left
ctccaacgacgcagagaaacac

SEQ ID NO: 121 - DNA, 22 bp, Artificial Sequence
  5_HPV18_R1_Right
ggattcaacggtttctggcacc

SEQ ID NO: 122 - DNA, 22 bp, Artificial Sequence
  11_HPV18_R2_Left
ttttggttcaggctggattgcg

SEQ ID NO: 123 - DNA, 22 bp, Artificial Sequence
  12_HPV18_R2_Right
agaatacacacagctgccaggt

SEQ ID NO: 124 - DNA, 22 bp, Artificial Sequence
  3259_HPV16_E6-E7
aaccggacagagcccattacaa

SEQ ID NO: 125 - DNA, 23 bp, Artificial Sequence
  3260_HPV16_E6-E7
agtcatatacctcacgtcgcagt

SEQ ID NO: 126 - DNA, 22 bp, Artificial Sequence
  3264_HPV16_L1
actggctttggtgctatggact

SEQ ID NO: 127 - DNA, 22 bp, Artificial Sequence
  3265_HPV16_L1
caaaccagccgctgtgtatctg

SEQ ID NO: 128 - DNA, 18 bp, Artificial Sequence
  HIV LTR Forward primer
gcctcaataaagcttgcc

SEQ ID NO: 129 - DNA, 21 bp, Artificial Sequence
  HIV LTR-Gag inter Reverse primer
ggcgccactgctagagatttt

SEQ ID NO: 130 - DNA, 15 bp, Artificial Sequence
  HIV LTR probe
aagtrgtgtgtgccc

SEQ ID NO: 131 - DNA, 19 bp, Artificial Sequence
  Human RPP30 gene Forward primer
agatttggacctgcgagcg

SEQ ID NO: 132 - DNA, 20 bp, Artificial Sequence
  Human RPP30 gene Reverse primer
gagcggctgtctccacaagt

SEQ ID NO: 133 - DNA, 23 bp, Artificial Sequence
  Human RPP30 gene Probe
ttctgacctgaaggctctgcgcg

SEQ ID NO: 134 - DNA, 20 bp, Artificial Sequence
  OAR12_62009791_62009791 BLV Oligo
tttcagagggcggagaaaca

SEQ ID NO: 135 - DNA, 20 bp, Artificial Sequence
  OAR2_248506820_248507220 BLV Oligo
tttagcaaacgccagggaac

SEQ ID NO: 136 - DNA, 20 bp, Artificial Sequence
  OAR5_60508711_60508719 BLV Oligo
tttcagagggcggagaaaca

SEQ ID NO: 137 - DNA, 20 bp, Artificial Sequence
  OAR12_62009791_62009791 Host Oligo
caccctgagcctccatacat

SEQ ID NO: 138 - DNA, 20 bp, Artificial Sequence
  OAR2_248506820_248507220 Host Oligo
gcgaatctctgtcttgctgg

SEQ ID NO: 139 - DNA, 21 bp, Artificial Sequence
  OAR5_60508711_60508719 Host Oligo
aactctatggctggaaggaca

SEQ ID NO: 140 - DNA, 20 bp, Artificial Sequence
  OARX_115780553_115780560 BLV Oligo
tttcagagggcggagaaaca

SEQ ID NO: 141 - DNA, 20 bp, Artificial Sequence
  OAR3_68849355_68850177 BLV Oligo
tttagcaaacgccagggaac

SEQ ID NO: 142 - DNA, 20 bp, Artificial Sequence
  OAR8_80138768_80138775 BLV Oligo
tttagcaaacgccagggaac

SEQ ID NO: 143 - DNA, 20 bp, Artificial Sequence
  OAR2_56698159_56698164 BLV Oligo
tttagcaaacgccagggaac

SEQ ID NO: 144 - DNA, 20 bp, Artificial Sequence
  OAR7_72660067_72660073 BLV Oligo
tttcagagggcggagaaaca

SEQ ID NO: 145 - DNA, 20 bp, Artificial Sequence
  OAR8_80151001_80151007 BLV Oligo
tttcagagggcggagaaaca

SEQ ID NO: 146 - DNA, 20 bp, Artificial Sequence
  OARX_115780553_115780560 Host Oligo
aggtggagatgatgtgtgca

SEQ ID NO: 147 - DNA, 20 bp, Artificial Sequence
  OAR3_68849355_68850177 Host Oligo
acctcacaccaaaacgaagc

SEQ ID NO: 148 - DNA, 20 bp, Artificial Sequence
  OAR8_80138768_80138775 Host Oligo
gtgacttgtttgcctccctg

SEQ ID NO: 149 - DNA, 20 bp, Artificial Sequence
  OAR2_56698159_56698164 Host Oligo
ttcatgtgcttccgtggttg

SEQ ID NO: 150 - DNA, 20 bp, Artificial Sequence
  OAR7_72660067_72660073 Host Oligo
agaggcctgagtgttttggt

SEQ ID NO: 151 - DNA, 20 bp, Artificial Sequence
  OAR8_80151001_80151007 Host Oligo
gacccacatcagttgccttc

SEQ ID NO: 152 - DNA, 20 bp, Artificial Sequence
  24_41573470_41573476 BLV Oligo
ggggctcgcaatcatatgtg

SEQ ID NO: 153 - DNA, 20 bp, Artificial Sequence
  22_48070162_48070168 BLV Oligo
ggggctcgcaatcatatgtg

SEQ ID NO: 154 - DNA, 20 bp, Artificial Sequence
  18_57045658_57045664 BLV Oligo
ggggctcgcaatcatatgtg

SEQ ID NO: 155 - DNA, 20 bp, Artificial Sequence
  18_61039250_61039250 BLV Oligo
ggggctcgcaatcatatgtg

SEQ ID NO: 156 - DNA, 20 bp, Artificial Sequence
  2_5529599_5529704 BLV Oligo
ggggctcgcaatcatatgtg

SEQ ID NO: 157 - DNA, 20 bp, Artificial Sequence
  2_5529599_5529704 BLV Oligo
agccctctggactcacaatc

SEQ ID NO: 158 - DNA, 20 bp, Artificial Sequence
  2_54238495_54238502 BLV Oligo
ggggctcgcaatcatatgtg

SEQ ID NO: 159 - DNA, 20 bp, Artificial Sequence
  2_54238495_54238502 BLV Oligo
agccctctggactcacaatc

SEQ ID NO: 160 - DNA, 20 bp, Artificial Sequence
  21_45410573_45410985 BLV Oligo
ggggctcgcaatcatatgtg

SEQ ID NO: 161 - DNA, 20 bp, Artificial Sequence
  29_41063804_41063804 BLV Oligo
agccctctggactcacaatc

SEQ ID NO: 162 - DNA, 20 bp, Artificial Sequence
  29_41063804_41063804 BLV Oligo
ggggctcgcaatcatatgtg

SEQ ID NO: 163 - DNA, 20 bp, Artificial Sequence
  3_87619443_87619450 BLV Oligo
agccctctggactcacaatc

SEQ ID NO: 164 - DNA, 20 bp, Artificial Sequence
  3_87619443_87619450 BLV Oligo
ggggctcgcaatcatatgtg

SEQ ID NO: 165 - DNA, 20 bp, Artificial Sequence
  1_150385145_150385351 BLV Oligo
agccctctggactcacaatc

SEQ ID NO: 166 - DNA, 20 bp, Artificial Sequence
  24_41573470_41573476 Host Oligo
cttgaactccgggaccttct

SEQ ID NO: 167 - DNA, 20 bp, Artificial Sequence
  22_48070162_48070168 Host Oligo
tcgaaaaggccaagtaccct

SEQ ID NO: 168 - DNA, 20 bp, Artificial Sequence
  18_57045658_57045664 Host Oligo
gatgggatgaggtcaggagg

SEQ ID NO: 169 - DNA, 20 bp, Artificial Sequence
  18_61039250_61039250 Host Oligo
acaggcaggatctttgtgga

SEQ ID NO: 170 - DNA, 20 bp, Artificial Sequence
  2_5529599_5529704 Host Oligo
gcacactgtcctgagatcca

SEQ ID NO: 171 - DNA, 20 bp, Artificial Sequence
  2_5529599_5529704 Host Oligo
ccagtgcatgcttaatcgct

SEQ ID NO: 172 - DNA, 20 bp, Artificial Sequence
  2_54238495_54238502 Host Oligo
aatccgttcatggttccgtg

SEQ ID NO: 173 - DNA, 20 bp, Artificial Sequence
  2_54238495_54238502 Host Oligo
gctgctaatttgactggcca

SEQ ID NO: 174 - DNA, 20 bp, Artificial Sequence
  21_45410573_45410985 Host Oligo
ctcggggagacagaaaacct

SEQ ID NO: 175 - DNA, 20 bp, Artificial Sequence
  29_41063804_41063804 Host Oligo
cttccctgctccatccctag

SEQ ID NO: 176 - DNA, 20 bp, Artificial Sequence
  29_41063804_41063804 Host Oligo
cagcttactccacccttcca

SEQ ID NO: 177 - DNA, 20 bp, Artificial Sequence
  3_87619443_87619450 Host Oligo
gcaagagaagagagtggggt

SEQ ID NO: 178 - DNA, 20 bp, Artificial Sequence
  3_87619443_87619450 Host Oligo
tctaatccccaagctgtgca

SEQ ID NO: 179 - DNA, 20 bp, Artificial Sequence
  1_150385145_150385351 Host Oligo
cgacaagcctggtaagatgc

SEQ ID NO: 180 - DNA, 20 bp, Artificial Sequence
  1_150385145_150385351 BLV Oligo
ggggctcgcaatcatatgtg

SEQ ID NO: 181 - DNA, 22 bp, Artificial Sequence
  2_124084208_124084213 BLV Oligo
gaggcatcgatagcatggtcct

SEQ ID NO: 182 - DNA, 22 bp, Artificial Sequence
  23_39892380_39892560 BLV Oligo
aaatctggggccacaattgcag

SEQ ID NO: 183 - DNA, 20 bp, Artificial Sequence
  27_36582809_36582809 BLV Oligo
ccaccctattgcttccctga

SEQ ID NO: 184 - DNA, 20 bp, Artificial Sequence
  27_36582809_36582809 BLV Oligo
ggcatgagtagctccagagt

SEQ ID NO: 185 - DNA, 22 bp, Artificial Sequence
  3_45576532_45576538 BLV Oligo
aaatctggggccacaattgcag

SEQ ID NO: 186 - DNA, 20 bp, Artificial Sequence
  4_100234239_100234246 BLV Oligo
agccctctggactcacaatc

SEQ ID NO: 187 - DNA, 22 bp, Artificial Sequence
  5_51456241_51456285 BLV Oligo
agcgaggagagtgagagtgaga

SEQ ID NO: 188 - DNA, 20 bp, Artificial Sequence
  1_150385145_150385351 Host Oligo
gtgggacggtgtttgaagtc

SEQ ID NO: 189 - DNA, 22 bp, Artificial Sequence
  2_124084208_124084213 Host Oligo
ttccccaagactttcccaggtc

SEQ ID NO: 190 - DNA, 22 bp, Artificial Sequence
  23_39892380_39892560 Host Oligo
tccagtggccgtgtatttgtct

SEQ ID NO: 191 - DNA, 20 bp, Artificial Sequence
  27_36582809_36582809 Host Oligo
ttcccttagcagtcaggtgg

SEQ ID NO: 192 - DNA, 20 bp, Artificial Sequence
  27_36582809_36582809 Host Oligo
aggccttcactctaaccgtt

SEQ ID NO: 193 - DNA, 22 bp, Artificial Sequence
  3_45576532_45576538 Host Oligo
tactgcccatcaccccttcatc

SEQ ID NO: 194 - DNA, 21 bp, Artificial Sequence
  4_100234239_100234246 Host Oligo
acaaaacagtcaaacagggct

SEQ ID NO: 195 - DNA, 22 bp, Artificial Sequence
  5_51456241_51456285 Host Oligo
cccctgcataaaatgaggcctg

SEQ ID NO: 196 - DNA, 22 bp, Artificial Sequence
  OAR25_25097056_25097063 BLV Oligo
agatttcagggaagtggggagc

SEQ ID NO: 197 - DNA, 20 bp, Artificial Sequence
  OARX_78143793_78143801 BLV Oligo
tggatgtggctggaatgtct

SEQ ID NO: 198 - DNA, 20 bp, Artificial Sequence
  OARX_78143793_78143801 BLV Oligo
aattacaggcggtcttggga

SEQ ID NO: 199 - DNA, 22 bp, Artificial Sequence
  OAR1_250672128_250672136 BLV Oligo
aaatgcccaaagaacgacggtc

SEQ ID NO: 200 - DNA, 22 bp, Artificial Sequence
  OAR2_242159705_242159712 BLV Oligo
cgaatcttccccatgcagcttc

SEQ ID NO: 201 - DNA, 22 bp, Artificial Sequence
  OAR8_80161637_80161982 BLV Oligo
aaatgcccaaagaacgacggtc

SEQ ID NO: 202 - DNA, 22 bp, Artificial Sequence
  OAR25_25097056_25097063 Host Oligo
tgccttctccgttcccaattct

SEQ ID NO: 203 - DNA, 20 bp, Artificial Sequence
  OARX_78143793_78143801 Host Oligo
caccagggaagtcttgttgc

SEQ ID NO: 204 - DNA, 20 bp, Artificial Sequence
  OARX_78143793_78143801 Host Oligo
cagcctcagagttccttcca

SEQ ID NO: 205 - DNA, 22 bp, Artificial Sequence
  OAR1_250672128_250672136 Host Oligo
agccttcacaagtcacctctcc

SEQ ID NO: 206 - DNA, 22 bp, Artificial Sequence
  OAR2_242159705_242159712 Host Oligo
gatgccctggaatggtttggtg

SEQ ID NO: 207 - DNA, 22 bp, Artificial Sequence
  OAR8_80161637_80161982 Host Oligo
tccagaagaggcaaagcaagga

SEQ ID NO: 208 - DNA, 22 bp, Artificial Sequence
  OAR10_34545991_34546003 BLV Oligo
aaatgcccaaagaacgacggtc

SEQ ID NO: 209 - DNA, 22 bp, Artificial Sequence
  OAR10_49266255_49266262 BLV Oligo
aaatgcccaaagaacgacggtc

SEQ ID NO: 210 - DNA, 22 bp, Artificial Sequence
  OAR10_34545991_34546003 Host Oligo
aagtcgagcaaggcacctatgt

SEQ ID NO: 211 - DNA, 22 bp, Artificial Sequence
  OAR10_49266255_49266262 Host Oligo
tggttgtgggtcatcatcgtct

SEQ ID NO: 212 - DNA, 22 bp, Artificial Sequence
  BTA8_37.3 Forward Oligo
ggctgcccttcactgagagtaa

SEQ ID NO: 213 - DNA, 22 bp, Artificial Sequence
  BTA21_18.6 Forward Oligo
tggctaagttccaccacactct

SEQ ID NO: 214 - DNA, 22 bp, Artificial Sequence
  BTA27_14.1 Forward Oligo
ggagcaaggtagaggggtgaag

SEQ ID NO: 215 - DNA, 22 bp, Artificial Sequence
  BTA8_37.3 Reverse Oligo
tttacccttggagtgtggcctt

SEQ ID NO: 216 - DNA, 22 bp, Artificial Sequence
  BTA21_18.6 Reverse Oligo
gggtcctctgtcctctgtcttc

SEQ ID NO: 217 - DNA, 22 bp, Artificial Sequence
  BTA27_14.1 Reverse Oligo
agagggaaatcacaccgaagca

SEQ ID NO: 218 - DNA, 22 bp, Artificial Sequence
  OAR1_86.0 Forward Oligo
gttgttgcatcttccggtcctg

SEQ ID NO: 219 - DNA, 22 bp, Artificial Sequence
  OAR3_39.2 Forward Oligo
tagcccagcaagagtctcccta

SEQ ID NO: 220 - DNA, 22 bp, Artificial Sequence
  OAR4_77.4 Forward Oligo
ttgatgtgaagagcctgtgagc

SEQ ID NO: 221 - DNA, 22 bp, Artificial Sequence
  OAR13_16.7 Forward Oligo
ggcttcaaacacacctcacctc

SEQ ID NO: 222 - DNA, 22 bp, Artificial Sequence
  OAR4_40.4 Forward Oligo
gagatggccgtgtgtgacaaag

SEQ ID NO: 223 - DNA, 22 bp, Artificial Sequence
  OAR5_73.0 Forward Oligo
tgaaagactcactgtggcccaa

SEQ ID NO: 224 - DNA, 22 bp, Artificial Sequence
  OAR13_66.0 Forward Oligo
actctctcccaacattgccctc

SEQ ID NO: 225 - DNA, 22 bp, Artificial Sequence
  OAR1_86.0 Reverse Oligo
ggagcctcaacgactctgctaa

SEQ ID NO: 226 - DNA, 22 bp, Artificial Sequence
  OAR3_39.2 Reverse Oligo
ccccttcatagcccactggaaa

SEQ ID NO: 227 - DNA, 22 bp, Artificial Sequence
  OAR4_77.4 Reverse Oligo
ccagcaactcagacaaaccagg

SEQ ID NO: 228 - DNA, 22 bp, Artificial Sequence
  OAR13_16.7 Reverse Oligo
aatgtgtagatggaggctgggc

SEQ ID NO: 229 - DNA, 22 bp, Artificial Sequence
  OAR4_40.4 Reverse Oligo
gctaacaaacgggtggcaaaga

SEQ ID NO: 230 - DNA, 22 bp, Artificial Sequence
  OAR5_73.0 Reverse Oligo
ctggggaagccaagcaaagatg

SEQ ID NO: 231 - DNA, 22 bp, Artificial Sequence
  OAR13_66.0 Reverse Oligo
attctggtggtctctgtggctc

SEQ ID NO: 232 - DNA, 23 bp, Artificial Sequence
  1_HPV18_R1_guide1 RNA target and Pam site
gtgctgcaaccgagcacgacagg

SEQ ID NO: 233 - DNA, 23 bp, Artificial Sequence
  2_HPV18_R1_guide2 RNA target and Pam site
gtgctcggttgcagcacgaatgg

SEQ ID NO: 234 - DNA, 23 bp, Artificial Sequence
  3_HPV18_R1_guide3 RNA target and Pam site
cgacgatttcacaacatagctgg

SEQ ID NO: 235 - DNA, 23 bp, Artificial Sequence
  8_HPV18_R2_guide4 RNA target and Pam site
attttagaggattggaactttgg

SEQ ID NO: 236 - DNA, 23 bp, Artificial Sequence
  9_HPV18_R2_guide5 RNA target and Pam site
tctgctatactgcttaaatttgg

SEQ ID NO: 237 - DNA, 23 bp, Artificial Sequence
  10_HPV18_R2_guide6 RNA target and Pam site
gcatcatattgcccaggtacagg

SEQ ID NO: 238 - DNA, 23 bp, Artificial Sequence
  3261_HPV16_E6-E7_G1  guide RNA target and Pam site
ctaattaacaaatcacacaacgg

SEQ ID NO: 239 - DNA, 23 bp, Artificial Sequence
  3262_HPV16_E6-E7_G2  guide RNA target and Pam site
gattccataatataaggggtcgg

SEQ ID NO: 240 - DNA, 23 bp, Artificial Sequence
  3263_HPV16_E6-E7_G3  guide RNA target and Pam site
gcaacaagacatacatcgaccgg

SEQ ID NO: 241 - DNA, 23 bp, Artificial Sequence
  3266_HPV16_L1_G1  guide RNA target and Pam site
ccacctataggggaacactgggg

SEQ ID NO: 242 - DNA, 23 bp, Artificial Sequence
  3267_HPV16_L1_G2  guide RNA target and Pam site
acctacctcaacacctacacagg

SEQ ID NO: 243 - DNA, 23 bp, Artificial Sequence
  3268_HPV16_L1_G3  guide RNA target and Pam site
taatagagaatgtatatctatgg

SEQ ID NO: 244 - DNA, 20 bp, Artificial Sequence
  5 prime accessory sequence
ttctaatacgactcactata

SEQ ID NO: 245 - DNA, 14 bp, Artificial Sequence
  3 prime accessory sequence
gttttagagctaga

SEQ ID NO: 246 - DNA, 22 bp, Artificial Sequence
  Tail
tttctgttggtgctgatattgc

SEQ ID NO: 247 - DNA, 22 bp, Artificial Sequence
  Tail
acttgcctgtcgctctatcttc